(19) United States
(12) Patent Application Publication
(10) Pub. No.: US 2019/0032057 A1
(43) Pub. Date: Jan. 31, 2019

(54) METHODS AND COMPOSITIONS FOR RNA-GUIDED TREATMENT OF HIV INFECTION

(71) Applicants: EXCISION BIOTHERAPEUTICS, INC., Andover, NJ (US); Temple University of the Commonwealth System of Higher Education, Philadelphia, PA (US)

(72) Inventors: Kamel KHALILI, Bala Cynwyd, PA (US); Thomas Malcolm, Andover, NJ (US)

(73) Assignees: EXCISION BIOTHERAPEUTICS, INC., Andover, NJ (US); Temple University of the Commonwealth System of Higher Education, Philadelphia, PA (US)

(21) Appl. No.: 16/072,589

(22) PCT Filed: Jan. 24, 2017
(86) PCT No.: PCT/US17/14667

§ 371 Date: Jul. 25, 2018

Related U.S. Application Data
(60) Provisional application No. 62/286,575, filed on Jan. 25, 2016.

(51) Int. Cl. C12N 15/113; C12N 15/10; C12Q 1/70; A61P 31/18; C12N 15/86; C12N 9/22; A61K 31/711; C12N 15/70

(52) U.S. Cl. CPC C12N 15/1132; C12N 15/102; C12Q 1/703; A61P 31/18; C12N 2310/20; C12N 9/22; A61K 31/711; C12N 15/70; C12N 2740/16021; C12N 15/86

(57) ABSTRACT

A method of inactivating a proviral DNA integrated into the genome of a host cell latently infected with a retrovirus by treating the host cell with a composition comprising a Clustered Regularly Interspaced Short Palindromic Repeat (CRISPR)-associated endonuclease, and two or more different guide RNAs (gRNAs), wherein each of the at least two gRNAs is complementary to a different target nucleic acid sequence in a long terminal repeat (LTR) in the proviral DNA, and inactivating the proviral DNA. A composition for use in inactivating a proviral DNA integrated into the genome of a host cell latently infected with a retrovirus including isolated nucleic acid sequences comprising a CRISPR-associated endonuclease and a guide RNA, wherein the guide RNA is complementary to a target sequence in a human immunodeficiency virus.

### STATEMENT REGARDING FEDERALLY SPONSORED RESEARCH

This invention was made with U.S. government support under grant numbers R01MH093271, R01NS087971, and P30MH092177 awarded by the National Institutes of Health. The U.S. government may have certain rights in the invention.

### BACKGROUND OF THE INVENTION

### 1. Technical Field

The present invention relates to compositions that specifically cleave target sequences in retroviruses, for example human immunodeficiency virus (HIV). Such compositions, which can include nucleic acids encoding a Clustered Regularly Interspace Short Palindromic Repeat (CRISPR) associated endonuclease and a guide RNA sequence complementary to a target sequence in a human immunodeficiency virus, can be administered to a subject having or at risk for contracting an HIV infection.

### 2. Background Art

For more than three decades since the discovery of HIV-1, AIDS remains a major public health problem affecting greater than 35.3 million people worldwide. AIDS remains incurable due to the permanent integration of HIV-1 into the host genome. Current therapy (highly active antiretroviral therapy or HAART) for controlling HIV-1 infection and impeding AIDS development profoundly reduces viral replication in cells that support HIV-1 infection and reduces plasma viremia to a minimal level. But HAART fails to suppress low level viral genome expression and replication in tissues and fails to target the latently-infected cells, for example, resting memory T cells, brain macrophages, microglia, and astrocytes, gut-associated lymphoid cells, that serve as a reservoir for HIV-1. Persistent HIV-1 infection is also linked to co-morbidities including heart and renal diseases, osteopenia, and neurological disorders. There is a continuing need for curative therapeutic strategies that target persistent viral reservoirs.

### SUMMARY OF THE INVENTION

Provided herein are compositions and methods relating to treatment and prevention of retroviral infections. The retrovirus can be a lentivirus, for example, a human immunodeficiency virus, a simian immunodeficiency virus, a feline immunodeficiency virus or a bovine immunodeficiency virus. The human immunodeficiency virus can be HIV-1 or HIV-2. In one embodiment, the compositions include a nucleic acid sequence comprising a sequence encoding a CRISPR-associated endonuclease and one or more guide RNAs, wherein the guide RNA is complementary to a target sequence in a human immunodeficiency virus. In some embodiments the nucleic acid is contained within an expression vector. In one embodiment, the compositions include a CRISPR-associated endonuclease polypeptide and one or more guide RNAs, wherein the guide RNA is complementary to a target sequence in a human immunodeficiency virus. Also provided are pharmaceutical compositions that include the nucleic acids, expression vectors, or polypeptides disclosed herein. Also provided herein are methods of treatment of the subject having or at risk for having a human immunodeficiency virus infection, wherein the method of treatment comprises administering to a subject a therapeutically effective amount of a composition comprising a vector encoding a CRISPR-associated endonuclease and one or more guide RNAs, wherein the guide RNA is complementary to a target sequence in the a human immunodeficiency virus. Also provided are methods of inactivating a retrovirus in a human cell by exposing the cell to a composition comprising an isolated nucleic acid encoding a gene editing complex comprising a CRISPR-associated endonuclease and one or more guide RNAs wherein the guide RNA is complementary to a target nucleic acid sequence in the retrovirus. The gene editing complex introduces one or more mutations into the proviral DNA. In some embodiments the mutations can include the deletion, which can comprise all or substantially all of the proviral DNA sequence. In another aspect, a kit comprising a measured amount of the compositions disclosed herein is also provided.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A, FIG. 1B, FIG. 1C, FIG. 1D, FIG. 1E, FIG. 1F, FIG. 1G, and FIG. 1H show that Cas9/LTR-gRNA suppresses HIV-1 reporter virus production in CHME5 microglial cells latently infected with HIV-1. FIG. 1A shows a representative gating diagram of EGFP flow cytometry shows a dramatic reduction in TSA-induced reactivation of latent pNL4-3-ΔGag-d2EGFP reporter virus by stably expressed Cas9 plus LTR-A or -B, vs. empty U6-driven gRNA expression vector (U6-CAG). FIG. 1B shows SURVEYOR Cel-I nuclease assay of PCR product (−453 to +43 within LTR) from selected LTR-A- or -B-expressing stable clones shows dramatic indel mutation patterns (arrows). FIG. 1C shows a PCR fragment analysis of a precise deletion of 190-bp region between LTRs A and B cutting sites (arrowhead and arrow in FIG. 1D), leaving 306-bp fragment (arrow in FIG. 1C) validated by TA-cloning and sequencing results. FIG. 1D discloses SEQ ID NOS 1-3, respectively, in order of appearance. FIG. 1E is a graph showing subcloning of LTR-A/B stable clones reveals complete loss of reporter reactivation determined by EGFP flow cytometry, and FIG. 1F shows elimination of pNL4-3-ΔGag-d2EGFP proviral genome detected by standard, and FIG. 1G shows real-time (1G) PCR amplification of genomic DNA for EGFP and HIV-1 Rev response element (RRE); β-actin is a DNA purification and loading control. FIG. 1H shows PCR genotyping of LTR-A/B subclones (#8, 13) using primers to amplify DNA fragment covering HIV-1 LTR U3/R/U5 regions (−411 to +129) shows indels (a, deletion; c, insertion) and “intact” or combined LTR (b).

FIG. 2A, FIG. 2B, and FIG. 2C show that Cas9/LTR-gRNA efficiently eradicates latent HIV-1 virus from U1 monocytic cells. FIG. 2A shows a diagram showing excision of HIV-1 entire genome in chromosome Xp11.4. HIV-1 integration sites were identified using a Genome-Walker link PCR kit. Left, analysis of PCR amplicon lengths using a primer pair (P1/P2) targeting chromosome X integration site-flanking sequence reveals elimination of the entire HIV-1 genome (9709-bp), leaving two fragments (833- and 670-bp). FIG. 2B shows TA cloning and sequencing of the LTR fragment (833-bp) showing the host genomic sequence (small letters, 226-bp) and the partial sequences (634−27=607 bp) of 5′-LTR (underlined using dashes) and 3′-LTR (first underlined section) with a 27-bp deletion around the LTR-A targeting site (second underlined section). Bottom, two indel alleles identified from 15 sequenced clonal amplicons. The 670-bp fragment consists of a host sequence (226-bp) and the remaining LTR sequence (634−190=444 bp) after 190-bp excision by simultaneous cutting at LTR-A and B target sites. The underlined and highlighted sequences indicate the gRNA LTR-A target site and PAM. FIG. 2B discloses SEQ ID NOS 4-13, respectively, in order of appearance. FIG. 2C shows a functional analysis of LTR-A/B-induced eradication of HIV-1 genome, showing substantial blockade of TSA/PMA reactivation-induced p24 virion release. U1 cells were transfected with pX260-LTRs-A, -B, or -A/B. After 2-week puromycin selection, cells were treated with TSA (250 nM)/PMA for 2 days before p24 Gag ELISA was performed.

FIG. 3A, FIG. 3B, and FIG. 3C show that stable expression of Cas9 plus LTR-A/B vaccinates TZM-bI cells against new HIV-1 virus infection. FIG. 3A shows immunohistochemistry (ICC) and Western blot (WB) analyses with anti-Flag antibody confirm the expression of Flag-Cas9 in TZM-bI stable clones puromycin (2 μg/ml)-selected for 2 weeks. FIG. 3B shows PCR genotyping of Cas9/LTR-A/B stable clones (c1-c7) reveals a close correlation of LTR excision with repression of LTR luciferase reporter activation. Fold changes represent TSA/PMA-induced levels over corresponding non-induction levels. FIG. 3C shows Cas9/LTR-A/B-expressing cells (c4) were infected with pseudotyped-pNL4-3-Nef-EGFP lentivirus at indicated multiplicity of infection (MOI) and infection efficiency measured by EGFP flow cytometry, 2 d post-infection. FIG. 3D shows phase-contrast/fluorescence micrographs show that LTR-A/B stable, but not control (U6-CAG; black) cells, are resistant to new infection (right panel) by pNL4-3-ΔE-EGFP HIV-1 reporter virus (gray).

FIG. 4A, FIG. 4B, FIG. 4C, and FIG. 4D illustrate the off-target effects of Cas9/LTR-A/B on the human genome. FIG. 4A is a SURVEYOR assay that shows no indel mutations in predicted/potential off-target regions in human TZM-bI and U1 cells. LTR-A on-target region (A) was used as a positive control and empty U6-CAG vector (U6) as a negative control. FIG. 4B shoes whole-genome sequencing of LTR-A/B stable TZM-bI subclone showing the numbers of called indels in the U6-CAG control and LTR-A/B samples, FIG. 4C shows detailed information on 10 called indels near gRNA target sites in both samples, and FIG. 4D shows distribution of off-target called indels. FIG. 4C discloses SEQ ID NOS 14-15, respectively, in order of appearance.

FIG. 5 shows the LTR U3 sequence of the integrated lentiviral LTR-firefly luciferase reporter identified by TA-cloning and sequencing of PCR product (−411 to −10) from the genomic DNA of human TZM-bI cells. The protospacer and PAM (NGG) sequences of 4 gRNAs (LTR-A to D) and the predicted binding sites of indicated transcription factors are highlighted. The precise cleavage sites are marked with scissors. +1 indicates the transcriptional start site. FIG. 5 discloses SEQ ID NO: 16.

FIG. 6A, FIG. 6B, and FIG. 6C show that LTR-C and LTR-D remarkably suppress TSA-induced reactivation of latent pNL4-3-ΔGag-d2EGFP virus in CHME5 microglia cells. FIG. 6A is a diagram schematically showing pNL4-3-ΔGag-d2EGFP vector containing Tat, Rev, Env, Vpu, and Nef with the reporter gene d2EGFP. FIG. 6B shows a SURVEYOR assay showing indel mutations in the on-target LTR genome of Cas9/LTR-D but not Cas9/LTR-C transfected cells. FIG. 6C shows a representative gating diagram of EGFP flow cytometry showing a dramatic reduction in TSA-induced reactivation of latent pNL4-3-ΔGag-d2EGFP reporter viruses by stable expression of Cas9/LTR-C or LTR-D as compared with empty U6-driven gRNA expression vector (U6-CAG).

FIG. 7A, FIG. 7B, FIG. 7C, FIG. 7D, FIG. 7E, and FIG. 7F show that both LTR-C and LTR-D induced indel mutations and significantly decreased constitutive and TSA/PMA-induced luciferase activity in TZM-bI cells stably incorporated with HIV-1 LTR-firefly luciferase reporter gene. FIG. 7A shows a functional luciferase reporter assay revealing a significant reduction of LTR reactivation by LTR-C, LTR-D or both. FIG. 7B shows a SURVEYOR assay showing indel mutation in LTR DNA (−453 to +43) induced by LTR-C and LTR-D (upper arrow). A combination of LTR-C and LTR-D generates a 194 bp fragment (lower arrow) resulting from the deletion of 302 bp region between LTR-C and LTR-D. FIG. 7C and FIG. 7D show Sanger sequencing of 30 clones validating the indel efficiency at 23% for LTR-C and 13% for LTR-D and example chromatograms showing insertion/deletion. FIG. 7C discloses SEQ ID NOS 17-25, respectively, in order of appearance. FIG. 7D discloses SEQ ID NOS 26-30, respectively, in order of appearance. FIG. 7E shows PCR-restriction fragment length polymorphism (RFLP) analysis using BsaJ I to cut 5 sites (96, 102, 372, 386, 482) of the PCR product covering −453 to +43 of LTR showing two major bands (96 bp and 270 bp) in the U6-CAG control sample, but an additional 372 bp band (upper arrow) after LTR-C-induced indel mutation at the 96/102 sites, a 290 bp band (middle arrow) after LTR-D-induced mutations at the 372 site and a 180 bp fragment (lower arrow) after LTR-C/D-induced excision. FIG. 7F shows chromatograms showing the deletion of a 302 bp fragment between LTR-C and LTR-D (top) and an additional 17 bp deletion (bottom). Red arrows indicate the junction sites. *P<0.05 indicates a significant decrease in LTR-C or LTR-D-mediated luciferase activation compared to U6-CAG control. FIG. 7F discloses SEQ ID NOS 31-32, respectively, in order of appearance.

FIG. 8A, FIG. 8B, and FIG. 8C illustrate the TA cloning and Sanger sequencing of PCR products from CHME5 subclones of LTR-A/B and empty U6-CAG control using primers covering HIV-1 LTR U3/R/U5 regions (−411 to +129). FIG. 8A shows possible combination of LTR-A and LTR-B cuts on both 5′- and 3′-LTRs generating potential fragments a-c as indicated. FIG. 8B shows blasting of fragment a (351 bp) showing 190 bp deletion between LTR-A and LTR-B cut sites. FIG. 8C shows a blast of fragment c (682 bp) showing a 175 bp insertion at the LTR-A cleavage site and a 27 bp deletion at the LTR-B cleavage site. FIG. 8C discloses SEQ ID NOS 33-34, respectively, in order of appearance.

FIG. 9A, FIG. 9B, FIG. 9C, and FIG. 9D demonstrate that Cas9/LTR-gRNA efficiently eradicates latent HIV-1 virus from U1 monocytic cells. FIG. 9A shows a Sanger sequencing of a 1.1 kb fragment from long-range PCR using a primer pair (T492/T493) targeting a chromosome 2 integration site-flanking sequence (small letters, 467-bp) reveals elimination of the entire HIV-1 genome (9709-bp), leaving combined 5′-LTR (underlined using dashes) and 3′-LTR with a 6-bp insertion (boxed) precisely at the third nucleotide from PAM (TGG) LTR-A targeting site (underlined) and a 4-bp deletion (nnnn). FIG. 9A discloses SEQ ID NO: 35. FIG. 9B is a representative DNA gel picture that shows specific eradication of the HIV-1 genome. NS, non-specific band. FIG. 9C is a graph and FIG. 9D is a graph showing quantitative PCR analysis using the primer pair targeting the Gag gene (T457/T458) shows 85% efficiency of entire HIV-1 genome eradication in Cas9/LTR-A/B-expressing U1 cells. U1 cells were transfected with pX260 empty vector (U6-CAG) or LTRs-A/B-encoding vectors. After 2-week puromycin selection, the cellular genomic DNAs were used for absolute quantitative qPCR analysis using spiked pNL4-3-ΔE-EGFP human genomic DNA as a standard. **P<0.01 indicates a significant decrease compared to the U6-CAG control.

FIG. 10A, FIG. 10B, and FIG. 10C show that Cas9/LTR gRNAs effectively eradicates HIV-1 provirus in J-Lat latently infected T cells. FIG. 10A shows functional analysis by EGFP flow cytometry reveals approximately 50% reduction of PMA and TNFα-induced reactivation of EGFP reporter viruses. FIG. 10B is a SURVEYOR assay that shows indel mutations (arrow) in the on-target LTR genome of Cas9/LTR-A/B transfected cells. J-Lat cells were transfected with pX260 empty vector or LTRs-A and -B. After 2-week puromycin selection, cells were treated with PMA or TNFα for 24 h. The genomic DNAs were subject to PCR using primers covering HIV-1 LTR U3/R/U5 regions (−411 to +129) and the SURVEYOR assay was performed. **P<0.01 indicates a significant decrease compared to the U6-CAG control. (10C) FIG. 10C shows a PCR fragment analysis using primers covering HIV-1 LTR (−374 to +43) shows a precise deletion of 190-bp region between LTRs A and B cutting sites, leaving 227-bp fragment (arrow). House-keeping gene β-actin serves as a DNA purification and loading control.

FIG. 11A, FIG. 11B, FIG. 11C, and FIG. 11D show that genome editing efficiency depends upon the presence of Cas9 and gRNAs. FIG. 11A shows PCR genotyping reveals the absence of a U6-driven LTR-A or LTR-B expression cassette and FIG. 11B shows absence/reduction of CMV-driven Cas9 DNA in puromycin-selected TZM-bI subclones without any indication of genomic editing. Genomic DNAs from indicated subclones were subject to conventional (FIG. 11A) or real-time (FIG. 116) PCR analyses using a primer pair covering U6 promoter (T351) and LTR-A (T354) or -B (T356), and targeting Cas9 (T477/T491). FIG. 11C and FIG. 11D show Cas9 protein expression is absent in ineffective TZM-bI subclones. FIG. 11C shows that the Flag-tagged Cas9 fusion protein was detected by Western blot (WB) and immunocytochemistry (ICC) with anti-Flag monoclonal antibody. HEK293T cell line stably expressing Flag-Cas9 was used as a positive control for WB. GAPDH serves as a protein loading control. Clone c6 contains Cas9 DNA but no Cas9 protein expression, suggesting a potential mechanism of epigenetic repression after puromycin selection. Clone c5 and c3 may represent a truncated Flag-Cas9 (tCas9). FIG. 11D shows that the nucleus was stained with Hoechst 33258.

FIG. 12A, FIG. 12B, FIG. 12C, and FIG. 12D demonstrate that stable expression of Cas9/LTR-A/B gRNAs in TZM-bI cells vaccinates against pseudotyped or native HIV-1 viruses. FIG. 12 shows that flow cytometry shows a significant reduction of native pNL4-3-ΔE-EGFP reporter virus infection efficiency in Cas9/LTR-A/B expressing TZM-bI subclones. Real-time PCR analysis reveals suppression or elimination of viral RNA as shown in FIG. 12B and DNA as shown in FIG. 12C by Cas9/LTR-A/B gRNAs. FIG. 12D shows that the firefly-luciferase luminescent assay demonstrates dramatic inhibition of virus infection-stimulated LTR promoter activity by Cas9/LTR-A/B gRNAs. The stable Cas9/LTR-A/B gRNA-expressing TZM-bI cells were infected for 2 hours with indicated native HIV-1 viruses, and washed twice with PBS. At 2 days post-infection, cells were collected, fixed and analyzed by flow cytometry for EGFP expression (in FIG. 12A), or lysed for total RNA extraction and RT-qPCR (in FIG. 12B), genomic DNA purification for qPCR (in FIG. 12C) and luminescence measurement (in FIG. 12D). *P<0.05 and **P<0.01 indicate significant decreases compared to the U6-CAG control.

FIG. 13 shows the predicted LTR gRNAs and their off-target numbers (100% match). The 5′-LTR sense and antisense sequences (SEQ ID NOS 79-111 and 112-141, respectively) (634 bp) of pHR′-CMV-LacZ lentiviral vector (AF105229) were utilized to search for Cas9/gRNA target sites containing a 20-bp guide sequence (protospacer) plus the protospacer adjacent motif sequence (NGG) using Jack Lin's CRISPR/Cas9 gRNA finder tool (http://spot.colorado.edu/^{˜}slin/cas9.html). Each gRNA plus NGG (AGG, TGG, GGG, CGG) was blasted against available human genomic and transcript sequences with 1000 aligned sequences being displayed. After pressing Control+F, copy/paste the target sequence (1-23 through 9-23 nucleotides) and find the number of genomic targets with 100% match. The number of off-targets for each searching was divided by 3 because of repeated genome library. The number shown indicates the sum of 4 searches (NGG). The top number (for example, for gRNA sequence (sense): 20, 19, 19, 17, 16, 15, 14, 13, 12) indicates the gRNA target sequences farthest from NGG. The sequence and off-target numbers for the selected LTR-A/B and LTR-C/D are highlighted red and green respectively.

FIG. 14 depicts the oligonucleotides for gRNA targeting sites and primers (SEQ ID NOS 36-78, respectively, in order of appearance) used for PCR and sequencing.

FIG. 15 shows the locations of predicted gRNA targeting sites of LTR-A and LTR-B and discloses “query Seq” sequences as SEQ ID NOS 142-252, and “ref Seq” sequences as SEQ ID NOS 253-363, all respectively, in order of appearance.

FIG. 16A, FIG. 16B, FIG. 16C, FIG. 16D, FIG. 16E, FIG. 16F, FIG. 16G, and FIG. 16H show that both LTR-C and LTR-D decreased constitutive and TSA/PMA-induced luciferase activity in TZMBl cells stably incorporated with HIV-1 LTR firefly luciferase reporter gene and combination induced precise genome excision. FIG. 16A shows that six gRNA targets were designed for the promoter region of HIV-LTR. FIG. 16A discloses SEQ ID NO: 16. TZMBl cells were cotransfected with Cas9-EGFP and chimera gRNA expression cassette (PCR products) by lipofectamine 2000. FIG. 16B is a graph showing that after 3 d, EGFP-positive cells were sorted through FACS and 2000 cells per group were collected for luciferase assay. FIG. 16B discloses SEQ ID: 31. FIG. 16C is a graph showing the population sorted cells were cultured for 2 d and treated with TSA/PMA for 1 d before luciferase assay. The single cells were sorted into 96-well plate and cultured till confluence for luciferase assay in the absence (shown in the graph of FIG. 16D) of TSA/PMA for 1 d or presence (shown in the graph of FIG. 1E) of TSA/PMA for 1 d. FIG. 16F and FIG. 16G show the PCR product from the population sorted cells were analyzed with Surveyor Cel-I nuclease assay and restriction fragment length polymorphism with Bsajl (FIG. 16G) showing mutation (FIG. 16F) or uncut (FIG. 16G) band (red arrow). A 200 bp fragment (FIG. 16F, FIG. 16G, black arrow) resulting from the deletion of 321 bp region between LTR-C and LTR-D as predicted (FIG. 16A, red arrowhead) was validated by TA-cloning and sequencing showing precise genomic excision (FIG. 16H). Sanger sequencing of PCR products from individual LTR-C and -D identified % and % indel mutation efficiency respectively. * p<0.05 indicates statistically significant reduction using a student's t test compared to the corresponding U6-CAG control. Protospace(E), Protospace(C), Protospace(A), Protospace(B), Protospace(D), and Protospace(F) correspond to SEQ ID NOS 365, 367, 369, 371, 373, and 375, respectively, in order of appearance.

FIG. 17A, FIG. 17B, FIG. 17C, FIG. 17D, FIG. 17E, FIG. 17F, FIG. 17G, and FIG. 17H show that Cas9/LTR-gRNA inhibited constitutive and inducible production of HIV-1 virus measured by EGFP flow cytometry in HIV-1 latently infected CHME5 microglia cell line. The pHR′ lentiviral vector containing Tat, Rev, Env, Vpu, and Nef with the reported gene d2EGFP was transduced into human fetal microglia cell line CHME5 and 400 bp deletion in U3 region of 3′-LTR is illustrated (shown in FIG. 17A). FIG. 17B is a graph showing transient transfection of Cas9/gRNA, Human HIV-1 LTR-A, B alone or combination decreased the intensity but not percentage of EGFP due to suppression of LTR promoter activity. FIG. 17C is a graph showing transient transfection of Cas9/gRNA, Human HIV-1 LTR-C, D alone or combination decreased the intensity but not percentage of EGFP due to suppression of LTR promoter activity. FIG. 17D and FIG. 18 are graphs showing that after antibiotic selection for 1-2 weeks, the percentage of EGFP cells was also reduced. FIG. 17F and FIG. 17G show the PCR product from the stable selected clones were analyzed with Surveyor Cel-I nuclease assay showing indel mutation dramatically in LTR-A and LTR-B but weakly in the combination of LTR-A/B (red arrow). A 331 bp fragment (shown in FIG. 17F and FIG. 17G, black arrow) resulting from the deletion of 190 bp region between LTR-A and LTR-B as predicted (FIG. 17H, red arrowhead) was validated by TA-cloning and sequencing showing precise genomic excision (FIG. 17H). FIG. 17H discloses SEQ ID NOS 1-3, respectively, in order of appearance.

FIG. 18 shows LTR of a representative HIV-1 sequence (SEQ ID NO: 376). The U3 region extends from nucleotide 1 to nucleotide 432 (SEQ ID NO: 377), the R region extends from nucleotide 432 to nucleotide 559 (SEQ ID NO: 378), and the U5 region extends from 560 to nucleotide 634 (SEQ ID NO: 379).

FIG. 19 shows LTR of a representative SIV sequence (SEQ ID NO: 380). The U3 region extends from nucleotide 1 to nucleotide 517 (SEQ ID NO: 381), the R region extends from nucleotide 518 to nucleotide 693 (SEQ ID NO: 382), and the U5 region extends from 694 to nucleotide 818 (SEQ ID NO: 383).

### DETAILED DESCRIPTION OF THE INVENTION

The present invention is based, in part, on our discovery that we could eliminate the integrated HIV-1 genome from HIV-1 infected cells by using the RNA-guided Clustered Regularly Interspace Short Palindromic Repeat (CRISPR)-Cas 9 nuclease system (Cas9/gRNA) in single and multiplex configurations. We identified highly specific targets within the HIV-1 LTR U3 region that were efficiently edited by Cas9/gRNA, inactivating viral gene expression and replication in latently-infected microglial, promonocytic and T cells. Cas9/gRNAs caused neither genotoxicity nor off-target editing to the host cells, and completely excised a 9709-bp fragment of integrated proviral DNA that spanned from its 5′- to 3′-LTRs. Furthermore, the presence of multiplex gRNAs within Cas9-expressing cells prevented HIV-1 infection. Our results suggest that Cas9/gRNA can be engineered to provide a specific, efficacious prophylactic and therapeutic approach against AIDS.

Accordingly, the invention features compositions comprising a nucleic acid encoding a CRISPR-associated endonuclease and a guide RNA that is complementary to a target sequence in a retrovirus, e.g., HIV, as well as pharmaceutical formulations comprising a nucleic acid encoding a CRISPR-associated endonuclease and a guide RNA that is complementary to a target sequence in HIV. Also featured are compositions comprising a CRISPR-associated endonuclease polypeptide and a guide RNA that is complementary to a target sequence in HIV, as well as pharmaceutical formulations comprising a CRISPR-associated endonuclease polypeptide and a guide RNA that is complementary to a target sequence in HIV.

Also featured are methods of administering the compositions to treat a retroviral infection, e.g., HIV infection, methods of eliminating viral replication, and methods of preventing HIV infection. The therapeutic methods described herein can be carried out in connection with other antiretroviral therapies (e.g., HAART).

The clinical course of HIV infection can vary according to a number of factors, including the subject's genetic background, age, general health, nutrition, treatment received, and the HIV subtype. In general, most individuals develop flu-like symptoms within a few weeks or months of infection. The symptoms can include fever, headache, muscle aches, rash, chills, sore throat, mouth or genital ulcers, swollen lymph glands, joint pain, night sweats, and diarrhea. The intensity of the symptoms can vary from mild to severe depending upon the individual. During the acute phase, the HIV viral particles are attracted to and enter cells expressing the appropriate CD4 receptor molecules. Once the virus has entered the host cell, the HIV encoded reverse transcriptase generates a proviral DNA copy of the HIV RNA and the pro-viral DNA becomes integrated into the host cell genomic DNA. It is this HIV provirus that is replicated by the host cell, resulting in the release of new HIV virions which can then infect other cells. The methods and compositions of the invention are generally and variously useful for excision of integrated HIV proviral DNA, although the invention is not so limited, and the compositions may be administered to a subject at any stage of infection or to an uninfected subject who is at risk for HIV infection.

The primary HIV infection subsides within a few weeks to a few months, and is typically followed by a long clinical “latent” period which may last for up to 10 years. The latent period is also referred to as asymptomatic HIV infection or chronic HIV infection. The subject's CD4 lymphocyte numbers rebound, but not to pre-infection levels and most subjects undergo seroconversion, that is, they have detectable levels of anti-HIV antibody in their blood, within 2 to 4 weeks of infection. During this latent period, there can be no detectable viral replication in peripheral blood mononuclear cells and little or no culturable virus in peripheral blood. During the latent period, also referred to as the clinical latency stage, people who are infected with HIV may experience no HIV-related symptoms, or only mild ones. But, the HIV virus continues to reproduce at very low levels. In subjects who have treated with anti-retroviral therapies, this latent period may extend for several decades or more. However, subjects at this stage are still able to transmit HIV to others even if they are receiving antiretroviral therapy, although anti-retroviral therapy reduces the risk of transmission. As noted above, anti-retroviral therapy does not suppress low levels of viral genome expression nor does it efficiently target latently infected cells such as resting memory T cells, brain macrophages, microglia, astrocytes and gut associated lymphoid cells.

Clinical signs and symptoms of AIDS (acquired immunodeficiency syndrome) appear as CD4 lymphocyte numbers decrease, resulting in irreversible damage to the immune system. Many patients also present with AIDS-related complications, including, for example, opportunistic infections such as tuberculosis, salmonellosis, cytomegalovirus, candidiasis, cryptococcal meningitis, toxoplasmosis, and cryptosporidiosis, as well as certain kinds of cancers, including for example, Kaposi's sarcoma, and lymphomas, as well as wasting syndrome, neurological complications, and HIV-associated nephropathy.

Compositions

The compositions of the invention include nucleic acids encoding a CRISPR-associated endonuclease, e.g., Cas9, and a guide RNA that is complementary to a target sequence in a retrovirus, e.g., HIV. In bacteria the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

The compositions of the invention can include a nucleic acid encoding a CRISPR-associated endonuclease. In some embodiments, the CRISPR-associated endonuclease can be a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Psuedomona aeruginosa, Escherichia coli, or other sequenced bacteria genomes and archaea, or other prokaryotic microorganisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins).

The Cas9 nuclease sequence can be a mutated sequence. For example the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks.

In addition to the wild type and variant Cas9 endonucleases previously described, the present invention also encompasses CRISPR systems including “enhanced specificity” S. pyogenes Cas9 variants (eSpCas9), which dramatically reduce off-target cleavage. These variants are engineered with alanine substitutions to neutralize positively charged sites in a groove that interacts with the non-target strand of DNA. This modification reduces interaction of Cas9 with the non-target strand, thereby encouraging re-hybridization between target and non-target strands. The effect of this modification is that cleavage requires more stringent Watson-Crick pairing between the gRNA and the target DNA strand, thus limiting off-target cleavage (Slaymaker, et al., 2015).

The present invention also includes another type of enhanced specificity Cas9 variant, “high fidelity” spCas9 variants (HF-Cas9) designed according to principles disclosed by Joung and colleagues (Kleinstiver, et al., 2016, which is incorporated herein in its entirety). Some of the HF-Cas9 variants were disclosed by Kleinstiver, et al. (2009). The present invention represents the first harnessing of these variants for the purposes of inactivating viral DNA and eliminating viral infection. The present invention also includes HF-Cas9 variants that have not been previously disclosed.

A gRNA-mediated complex between Cas9 and a target DNA involves numerous contacts between Cas9 and the target DNA, including hydrogen bonds and hydrophobic base stacking. The HF-Cas9 variants of the present invention arise from the concept that these contacts possess more energy than required to stabilize the complex for DNA cleavage by Cas9. This permits a stable complex to form even when mismatches partially interrupt the binding of a gRNA to its target sequence. The HF-Cas9 variants include amino acid substitutions that disrupt some of the contacts between Cas9 and the target DNA. The disruption decreases contact energy to the point that a stable complex will only result from perfect or near perfect matches between a gRNA and its target sequence.

One of the HF-Cas9 variants disclosed by Kleinstiver, et al. includes four alanine substitutions, N497A, R661A, Q695A, and Q926A. These substitutions all reduce residue-to-base hydrogen bonding, with N497A also affecting hydrogen bonding through the peptide backbone. This variant was found to possess on-target cleavage activity comparable to that of wild-type Cas9, but markedly reduced off-target cleavage at deliberately mismatched sites, in experiments with cultured human osteosarcoma cells. This variant is included in the present invention, as variant 1 (SpCas9 N497A, R661A, Q695A, Q926A, in TABLE 1. Three additional variants were tested. They all included the previously mentioned four substitutions, N497A, R661A, Q695A, and Q926A of spCas9-HF-1 and, in addition, included a fifth substitution, D1135E, L169A, or Y450A. The additional substitutions were all of amino acids involved in base pair stacking. These variants exhibited on-target activities comparable to those of wild type Cas9, with off-target cleavage reduced to levels even lower than those observed for variant 1. The present invention includes these variants in the context of antiviral CRISPR/Cas systems. In TABLE 1, these are designated as variant 2 (SpCas9 N497A, R661A, Q695A, Q926A, D1135E), variant 3 (SpCas9 N497A, R661A, Q695A, Q926A, L169A), and variant 4 (SpCas9 N497A, R661A, Q695A, Q926A, Y450A).

Kleinstiver, et al. also disclosed a variant which included only three of the substitutions of variant 1, namely R661A, Q695A, and Q926A. This variant produced on-target cleavage comparable to that seen with wild type Cas9, but was not tested for off target effects. Because it is predicted to reduce off-target effects, this 3-substitution variant is included in the present invention, as variant 13 (SpCas9 R661A, Q695A, Q926A) in TABLE 1.

The present invention also includes Cas9 variants not disclosed by Kleinstiver, et al. These variants are all predicted to possess on-target activities comparable to that of wild type Cas9, with reduced off-target activities relative to wild type Cas9.

Three of the previously undisclosed variants, 14, 15, and 16, represent the addition of a fourth substitution to variant 13. Variant 14 includes a D113E substitution, and is designated SpCas9 R661A, Q695A, Q926A, D1135E. Variant 15 includes an L169A substitution, and is designated SpCas9 R661A, Q695A, Q926A, L169A. The L169A mutation affects hydrophobic base pair stacking. Variant 16 includes a Y450A substitution, and is designated SpCas9 R661A, Q695A, Q926A, Y450A. The Y540A substitution affects hydrophobic base pair stacking.

Two of the variants, 5 and 17, represent the addition of the substitution M495A to, respectively, variants 1 and 13. Variant 5 is therefore designated SpCas9 N497A, R661A, Q695A, Q926A, M495A, and variant 17 is designated SpCas9 R661A, Q695A, Q926A, M495A. The M495A substitution affects residue to base hydrogen bonding and hydrogen bonding through the peptide backbone.

Two of the variants, 6 and 18, represent the addition of the substitution M695A to, respectively, variants 1 and 13. Variant 6 is therefore designated SpCas9 N497A, R661A, Q695A, Q926A, M695A, and variant 18 is designated SpCas9 R661A, Q695A, Q926A, M694A. The M694A substitution affects hydrophobic base pair stacking.

Two of the variants, 7 and 19, represent the addition of the substitution H698A to, respectively, variants 1 and 13. Variant 7 is therefore designated SpCas9 N497A, R661A, Q695A, Q926A, H698A, and variant 19 is designated SpCas9 R661A, Q695A, Q926A, H698A. The H698A substitution affects hydrophobic base pair stacking.

Five of the variants, 8-12, represent the addition of a sixth substitution to the five-substitution variant 2. The added substitutions are, respectively, L169A, Y450A, M495A, M694A, and M698A. Therefore, variant 8 is designated SpCas9 N497A, R661A, Q695A, Q926A, D1135E, L169A; variant 9 is designated SpCas9 N497A, R661A, Q695A, Q926A, D1135E, Y450A; variant 10 is designated SpCas9 N497A, R661A, Q695A, Q926A, D1135E, Y495A; variant 11 is designated SpCas9 N497A, R661A, Q695A, Q926A, D1135E, M694A; and variant 12 is designated SpCas9 N497A, R661A, Q695A, Q926A, D1135E, M698A.

Four of the variants, 20-23, represent the addition of a fifth substitution to the four-substitution variant 13. The added substitutions are, respectively, L169A, Y450A, M495A, and M694A. Therefore, variant 20 is designated SpCas9 R661A, Q695A, Q926A, D1135E, L169A; variant 21 is designated SpCas9 R661A, Q695A, Q926A, D1135E, Y450A; variant 22 is designated SpCas9 R661A, Q695A, Q926A, D1135E, M495A; and variant 23 is designated SpCas9 R661A, Q695A, Q926A, D1135E, M694A.

TABLE 1

| Variant | Four Alanine Substitution | |
|---|---|---|
| No. | Mutants (compared to WT Cas9) | Tested* |
| 1 | SpCas9 N497A, R661A, Q695A, Q926A | YES |
| 2 | SpCas9 N497A, R661A, Q695A, Q926A + | YES |
| | D1135E | |
| 3 | SpCas9 N497A, R661A, Q695A, Q926A + | YES |
| | L169A | |
| 4 | SpCas9 N497A, R661A, Q695A, Q926A + | YES |
| | Y450A | |
| 5 | SpCas9 N497A, R661A, Q695A, Q926A + | Predicted |
| | M495A | |
| 6 | SpCas9 N497A, R661A, Q695A, Q926A + | Predicted |
| | M694A | |
| 7 | SpCas9 N497A, R661A, Q695A, Q926A + | Predicted |
| | H698A | |
| 8 | SpCas9 N497A, R661A, Q695A, Q926A + | Predicted |
| | D1135E + L169A | |
| 9 | SpCas9 N497A, R661A, Q695A, Q926A + | Predicted |
| | D1135E + Y450A | |
| 10 | SpCas9 N497A, R661A, Q695A, Q926A + | Predicted |
| | D1135E + M495A | |
| 11 | SpCas9 N497A, R661A, Q695A, Q926A + | Predicted |
| | D1135E + M694A | |
| 12 | SpCas9 N497A, R661A, Q695A, Q926A + | Predicted |
| | D1135E + M698A | |
| | Three Alanine Substitution | |
| | Mutants (compared to WT Cas9) | Tested* |
| 13 | SpCas9 R661A, Q695A, Q926A | No (on |
| | | target |
| | | only) |
| 14 | SpCas9 R661A, Q695A, Q926A + D1135E | Predicted |
| 15 | SpCas9 R661A, Q695A, Q926A + L169A | Predicted |
| 16 | SpCas9 R661A, Q695A, Q926A + Y450A | Predicted |
| 17 | SpCas9 R661A, Q695A, Q926A + M495A | Predicted |
| 18 | SpCas9 R661A, Q695A, Q926A + M694A | Predicted |
| 19 | SpCas9 R661A, Q695A, Q926A + H698A | Predicted |
| 20 | SpCas9 R661A, Q695A, Q926A + D1135E + | Predicted |
| | L169A | |
| 21 | SpCas9 R661A, Q695A, Q926A + D1135E + | Predicted |
| | Y450A | |
| 22 | SpCas9 R661A, Q695A, Q926A + D1135E + | Predicted |
| | M495A | |
| 23 | SpCas9 R661A, Q695A, Q926A + D1135E + | Predicted |
| | M694A | |

Tested beyond on-targeted eGFP disruption assays (Kleinstiver, et al., 2009).

It will be understood that the variants disclosed in TABLE 1 can themselves contain further amino acid substitutions and other mutations to further alter their function. For example, Cas9 sequence can be mutated to behave as “nickase”, which nicks rather than cleaves DNA, to yield single-stranded breaks. In Cas9, nickase activity is accomplished by mutations in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks (Sander and Joung, 2014). The nucleic acid sequence of any or all of the HF-Cas9 can be codon optimized for efficient expression in mammalian cells, i.e., “humanized”.

In some embodiments, compositions of the invention can include a CRISPR-associated endonuclease polypeptide encoded by any of the nucleic acid sequences described above. The terms “peptide,” “polypeptide,” and “protein” are used interchangeably herein, although typically they refer to peptide sequences of varying sizes. We may refer to the amino acid-based compositions of the invention as “polypeptides” to convey that they are linear polymers of amino acid residues, and to help distinguish them from full-length proteins. A polypeptide of the invention can “constitute” or “include” a fragment of a CRISPR-associated endonuclease, and the invention encompasses polypeptides that constitute or include biologically active variants of a CRISPR-associated endonuclease. It will be understood that the polypeptides can therefore include only a fragment of a CRISPR-associated endonuclease (or a biologically active variant thereof) but may include additional residues as well. Biologically active variants will retain sufficient activity to cleave target DNA.

The bonds between the amino acid residues can be conventional peptide bonds or another covalent bond (such as an ester or ether bond), and the polypeptides can be modified by amidation, phosphorylation or glycosylation. A modification can affect the polypeptide backbone and/or one or more side chains. Chemical modifications can be naturally occurring modifications made in vivo following translation of an mRNA encoding the polypeptide (e.g., glycosylation in a bacterial host) or synthetic modifications made in vitro. A biologically active variant of a CRISPR-associated endonuclease can include one or more structural modifications resulting from any combination of naturally occurring (i.e., made naturally in vivo) and synthetic modifications (i.e., naturally occurring or non-naturally occurring modifications made in vitro). Examples of modifications include, but are not limited to, amidation (e.g., replacement of the free carboxyl group at the C-terminus by an amino group); biotinylation (e.g., acylation of lysine or other reactive amino acid residues with a biotin molecule); glycosylation (e.g., addition of a glycosyl group to either asparagines, hydroxylysine, serine or threonine residues to generate a glycoprotein or glycopeptide); acetylation (e.g., the addition of an acetyl group, typically at the N-terminus of a polypeptide); alkylation (e.g., the addition of an alkyl group); isoprenylation (e.g., the addition of an isoprenoid group); lipoylation (e.g. attachment of a lipoate moiety); and phosphorylation (e.g., addition of a phosphate group to serine, tyrosine, threonine or histidine).

One or more of the amino acid residues in a biologically active variant may be a non-naturally occurring amino acid residue. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins).

Alternatively, or in addition, one or more of the amino acid residues in a biologically active variant can be a naturally occurring residue that differs from the naturally occurring residue found in the corresponding position in a wildtype sequence. In other words, biologically active variants can include one or more amino acid substitutions. We may refer to a substitution, addition, or deletion of amino acid residues as a mutation of the wild-type sequence. As noted, the substitution can replace a naturally occurring amino acid residue with a non-naturally occurring residue or just a different naturally occurring residue. Further the substitution can constitute a conservative or non-conservative substitution. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The polypeptides that are biologically active variants of a CRISPR-associated endonuclease can be characterized in terms of the extent to which their sequence is similar to or identical to the corresponding wild-type polypeptide. For example, the sequence of a biologically active variant can be at least or about 80% identical to corresponding residues in the wild-type polypeptide. For example, a biologically active variant of a CRISPR-associated endonuclease can have an amino acid sequence with at least or about 80% sequence identity (e.g., at least or about 85%, 90%, 95%, 97%, 98%, or 99% sequence identity) to a CRISPR-associated endonuclease or to a homolog or ortholog thereof.

A biologically active variant of a CRISPR-associated endonuclease polypeptide will retain sufficient biological activity to be useful in the present methods. The biologically active variants will retain sufficient activity to function in targeted DNA cleavage. The biological activity can be assessed in ways known to one of ordinary skill in the art and includes, without limitation, in vitro cleavage assays or functional assays.

Polypeptides can be generated by a variety of methods including, for example, recombinant techniques or chemical synthesis. Once generated, polypeptides can be isolated and purified to any desired extent by means well known in the art. For example, one can use lyophilization following, for example, reversed phase (preferably) or normal phase HPLC, or size exclusion or partition chromatography on polysaccharide gel media such as Sephadex G-25. The composition of the final polypeptide may be confirmed by amino acid analysis after degradation of the peptide by standard means, by amino acid sequencing, or by FAB-MS techniques. Salts, including acid salts, esters, amides, and N-acyl derivatives of an amino group of a polypeptide may be prepared using methods known in the art, and such peptides are useful in the context of the present invention.

The compositions of the invention include sequence encoding a guide RNA (gRNA) comprising a sequence that is complementary to a target sequence in a retrovirus. The retrovirus can be a lentivirus, for example, a human immunodeficiency virus, a simian immunodeficiency virus, a feline immunodeficiency virus or a bovine immunodeficiency virus. The human immunodeficiency virus can be HIV-1 or HIV-2. The target sequence can include a sequence from any HIV, for example, HIV-1 and HIV-2, and any circulating recombinant form thereof. The genetic variability of HIV is reflected in the multiple groups and subtypes that have been described. A collection of HIV sequences is compiled in the Los Alamos HIV databases and compendiums. The methods and compositions of the invention can be applied to HIV from any of those various groups, subtypes, and circulating recombinant forms. These include for example, the HIV-1 major group (often referred to as Group M) and the minor groups, Groups N, O, and P, as well as but not limited to, any of the following subtypes, A, B, C, D, F, G, H, J and K. or group (for example, but not limited to any of the following Groups, N, O and P) of HIV. The methods and compositions can also be applied to HIV-2 and any of the A, B, C, F or G clades (also referred to as “subtypes” or “groups”), as well as any circulating recombinant form of HIV-2.

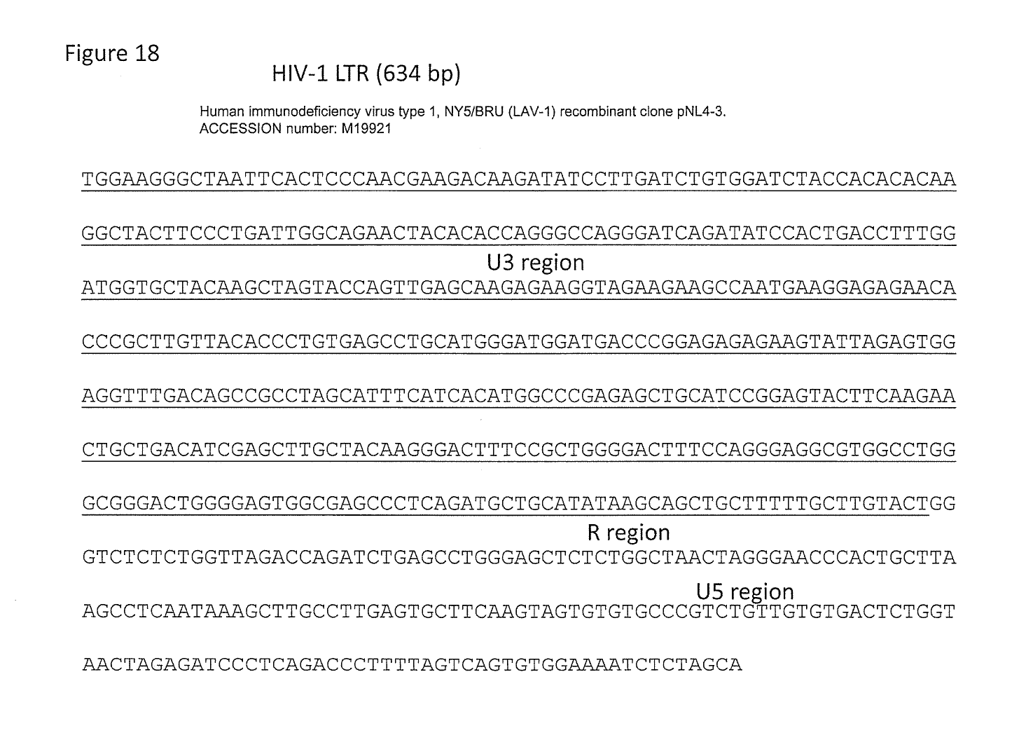

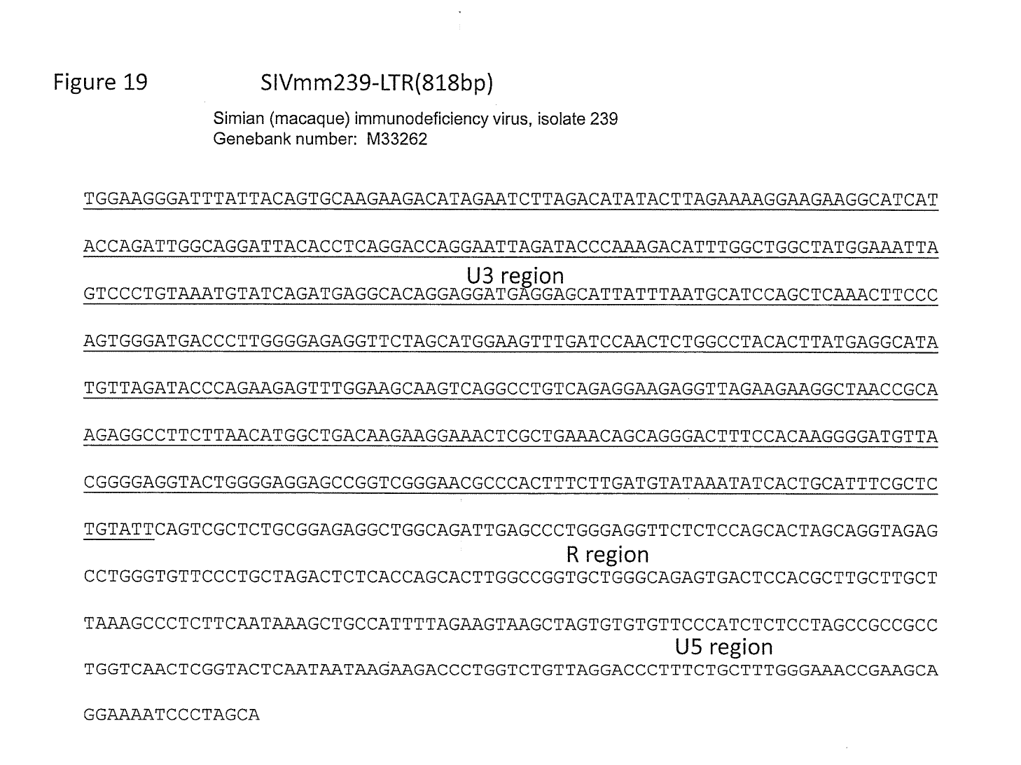

The guide RNA can be a sequence complimentary to a coding or a non-coding sequence. For example, the guide RNA can be an HIV sequence, such as a long terminal repeat (LTR) sequence, a protein coding sequence, or a regulatory sequence. In some embodiments, the guide RNA comprises a sequence that is complementary to an HIV long terminal repeat (LTR) region. The HIV-1 LTR is approximately 640 bp in length. An exemplary HIV-1 LTR is the sequence of SEQ ID NO: 376. An exemplary SIV LTR is the sequence of SEQ ID NO: 380. HIV-1 long terminal repeats (LTRs) are divided into U3, R and U5 regions. Exemplary HIV-1 LTR U3, R and U5 regions are SEQ ID NOs: 377, 378 and 379, respectively. Exemplary SIV LTR U3, R and U5 regions are SEQ ID NOs: 381, 382, and 383, respectively. The configuration of the U1, R, U5 regions for exemplary HIV-1 and SIV sequences are shown in FIGS. 18 and 19, respectively. LTRs contain all of the required signals for gene expression and are involved in the integration of a provirus into the genome of a host cell. For example, the basal or core promoter, a core enhancer and a modulatory region is found within U3 while the transactivation response element is found within R. In HIV-1, the U5 region includes several sub-regions, for example, TAR or trans-acting responsive element, which is involved in transcriptional activation; Poly A, which is involved in dimerization and genome packaging; PBS or primer binding site; Psi or the packaging signal; DIS or dimer initiation site

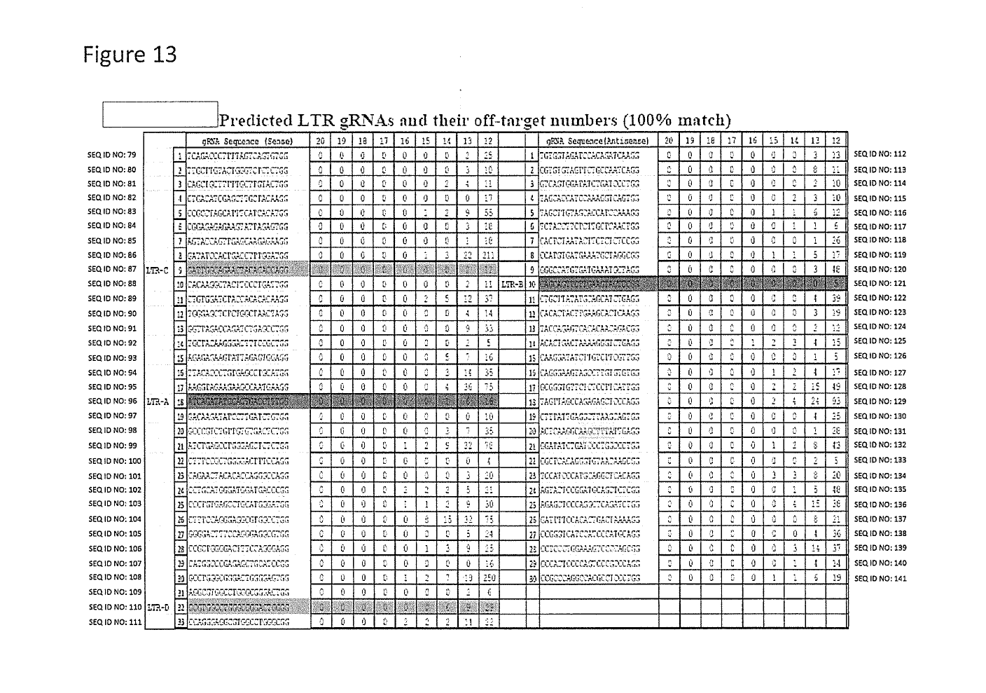

Useful guide sequences are complementary to the U3, R, or U5 region of the LTR. Exemplary guide RNA sequences that target the U3 region of HIV-1 are shown in FIG. 13. A guide RNA sequence can comprise, for example, a sequence complementary to the target protospacer sequence of:

(SEQ ID NO: 96)
LTR A: ATCAGATATCCACTGACCTTTGG,
(SEQ ID NO: 121)
LTR B: CAGCAGTTCTTGAAGTACTCCGG,
(SEQ ID NO: 87)
LTR C: GATTGGCAGAACTACACACCAGG,
or
(SEQ ID NO: 110)
LTR D: GCGTGGCCTGGGCGGGACTGGGG.

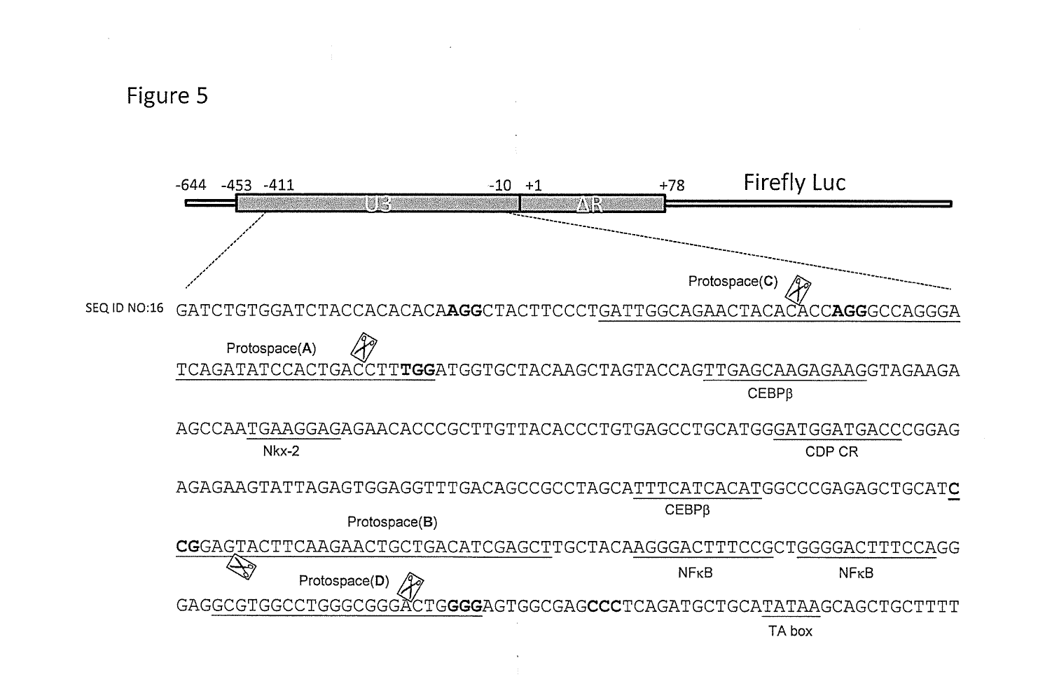

The locations of LTR A (SEQ ID NO: 96), LTR B (SEQ ID NO: 121), LTR C (SEQ ID NO: 87) and LTR D (SEQ ID NO: 110) within the U3 (SEQ ID NO: 16) region are shown FIG. 5. Additional exemplary guide RNA sequences that target the U3 region are listed in the table shown in FIG. 13 and can have the sequence of any of SEQ ID NOs: 79-111 and SEQ ID NOs: 111-141. In some embodiments, the guide sequence can comprise a sequence having 95% identity to any of SEQ ID NOs: 79-111 and SEQ ID NOs: 111-141. Thus, a guide RNA sequence can comprise, for example, a sequence having 95% identity to a sequence complementary to the target protospacer sequence of:

(SEQ ID NO: 96)
LTR A: ATCAGATATCCACTGACCTTTGG,
(SEQ ID NO: 121)
LTR B: CAGCAGTTCTTGAAGTACTCCGG,
(SEQ ID NO: 87)
LTR C GATTGGCAGAACTACACACCAGG,
or
(SEQ ID NO: 110)
LTR D: GCGTGGCCTGGGCGGGACTGGGG.

We may also be refer to the guide RNA sequence as a spacer, e.g., spacer (A), spacer (B), spacer (C), and spacer(D).

The guide RNA sequence can be complementary to a sequence found within an HIV-1 U3, R, or U5 region reference sequence or consensus sequence. The invention is not so limiting however, and the guide RNA sequences can be selected to target any variant or mutant HIV sequence. In some embodiments, more than one guide RNA sequence is employed, for example a first guide RNA sequence and a second guide RNA sequence, with the first and second guide RNA sequences being complimentary to target sequences in any of the above mentioned retroviral regions. In some embodiments, the guide RNA can include a variant sequence or quasi-species sequence. In some embodiments, the guide RNA can be a sequence corresponding to a sequence in the genome of the virus harbored by the subject undergoing treatment. Thus for example, the sequence of the particular U3, R, or U5 region in the HIV virus harbored by the subject can be obtained and guide RNAs complementary to the patient's particular sequences can be used.

In some embodiments, the guide RNA can be a sequence complimentary to a protein coding sequence, for example, a sequence encoding one or more viral structural proteins, (e.g., gag, pol, env and tat). Thus, the sequence can be complementary to sequence within the gag polyprotein, e.g., MA (matrix protein, p17); CA (capsid protein, p24); SP1 (spacer peptide 1, p2); NC (nucleocapsid protein, p7); SP2 (spacer peptide 2, p1) and P6 protein; pol, e.g., reverse transcriptase (RT) and RNase H, integrase (IN), and HIV protease (PR); env, e.g., gp160, or a cleavage product of gp160, e.g., gp120 or SU, and gp41 or TM; or tat, e.g., the 72-amino acid one-exon Tat or the 86-101 amino-acid two-exon Tat. In some embodiments, the guide RNA can be a sequence complementary to a sequence encoding an accessory protein, including, for example, vif, nef (negative factor) vpu (Virus protein U) and tev.

In some embodiments, the sequence can be a sequence complementary to a structural or regulatory element, for example, an LTR, as described above; TAR (Target sequence for viral transactivation), the binding site for Tat protein and for cellular proteins, consists of approximately the first 45 nucleotides of the viral mRNAs in HIV-1 (or the first 100 nucleotides in HIV-2) forms a hairpin stem-loop structure; RRE (Rev responsive element) an RNA element encoded within the env region of HIV-1, consisting of approximately 200 nucleotides (positions 7710 to 8061 from the start of transcription in HIV-1, spanning the border of gp120 and gp41); PE (Psi element), a set of 4 stem-loop structures preceding and overlapping the Gag start codon; SLIP, a TTTTTT “slippery site”, followed by a stem-loop structure; CRS (Cis-acting repressive sequences); INS Inhibitory/Instability RNA sequences) found for example, at nucleotides 414 to 631 in the gag region of HIV-1.

The guide RNA sequence can be a sense or anti-sense sequence. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neiseria menigiditis requires 5′-NNNNGATT). The specific sequence of the guide RNA may vary, but, regardless of the sequence, useful guide RNA sequences will be those that minimize off-target effects while achieving high efficiency and complete ablation of the genomically integrated HIV-1 provirus. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites; avoiding transcription factor binding sites within the HIV-1 LTR promoter (potentially conserved in the host genome); selection of LTR-A- and -B-directed, 30-bp gRNAs and also pre-crRNA system reflecting the original bacterial immune mechanism to enhance specificity/efficiency vs. 20-bp gRNA-, chimeric crRNA-tracRNA-based system and WGS, Sanger sequencing and SURVEYOR assay, to identify and exclude potential off-target effects.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs, for example any combination of sequences in U3, R, or U5. In some embodiments, combinations of LTR A, LTR B, LTR C and LTR D can be used. In some embodiments, combinations of any of the sequences LTR A (SEQ ID NO: 96), LTR B (SEQ ID NO: 121), LTR C (SEQ ID NO: 87), and LTR D (SEQ ID NO: 110), can be used. In some embodiments, any combinations of the sequences having the sequence of SEQ ID NOs: 79-111 and SEQ ID NOs: 111-141 can be used. When the compositions are administered in an expression vector, the guide RNAs can be encoded by a single vector. Alternatively, multiple vectors can be engineered to each include two or more different guide RNAs. Useful configurations will result in the excision of viral sequences between cleavage sites resulting in the ablation of HIV genome or HIV protein expression. Thus, the use of two or more different guide RNAs promotes excision of the viral sequences between the cleavage sites recognized by the CRISPR endonuclease. The excised region can vary in size from a single nucleotide to several thousand nucleotides. Exemplary excised regions are described in the examples.

When the compositions are administered as a nucleic acid or are contained within an expression vector, the CRISPR endonuclease can be encoded by the same nucleic acid or vector as the guide RNA sequences. Alternatively or in addition, the CRISPR endonuclease can be encoded in a physically separate nucleic acid from the guide RNA sequences or in a separate vector.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N.sup.6N-methyladenosine; N⁶, N⁶-dimethyladenosine; N⁶,2′-O-trimethyladenosine; 2-methylthio-N⁶N-isopentenyladenosine; N⁶-(cis-hydroxyisopentenyl)-adenosine; 2-methylthio-N⁶-(cis--hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N²,N²-dimethyl guanosine; N², 2′-O-dimethyl guanosine; N², N², 2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

We may use the terms “nucleic acid” and “polynucleotide” interchangeably to refer to both RNA and DNA, including cDNA, genomic DNA, synthetic DNA, and DNA (or RNA) containing nucleic acid analogs, any of which may encode a polypeptide of the invention and all of which are encompassed by the invention. Polynucleotides can have essentially any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode a fragment of a naturally occurring Cas9 or a biologically active variant thereof and a guide RNA where in the guide RNA is complementary to a sequence in HIV.

An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a Cas9-encoding DNA (in accordance with, for example, the formula above).

Two nucleic acids or the polypeptides they encode may be described as having a certain degree of identity to one another. For example, a Cas9 protein and a biologically active variant thereof may be described as exhibiting a certain degree of identity. Alignments may be assembled by locating short Cas9 sequences in the Protein Information Research (PIR) site, followed by analysis with the “short nearly identical sequences.” Basic Local Alignment Search Tool (BLAST) algorithm on the NCBI website.

As used herein, the term “percent sequence identity” refers to the degree of identity between any given query sequence and a subject sequence. For example, a naturally occurring Cas9 can be the query sequence and a fragment of a Cas9 protein can be the subject sequence. Similarly, a fragment of a Cas9 protein can be the query sequence and a biologically active variant thereof can be the subject sequence.

To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chenna et al., Nucleic Acids Res. 31:3497-3500, 2003.

ClustalW calculates the best match between a query and one or more subject sequences and aligns them so that identities, similarities and differences can be determined. Gaps of one or more residues can be inserted into a query sequence, a subject sequence, or both, to maximize sequence alignments. For fast pair wise alignment of nucleic acid sequences, the following default parameters are used: word size: 2; window size: 4; scoring method: percentage; number of top diagonals: 4; and gap penalty: 5. for multiple alignments of nucleic acid sequences, the following parameters are used: gap opening penalty: 10.0; gap extension penalty: 5.0; and weight transitions: yes. For fast pair wise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The nucleic acids and polypeptides described herein may be referred to as “exogenous”. The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

Recombinant constructs are also provided herein and can be used to transform cells in order to express Cas9 and/or a guide RNA complementary to a target sequence in HIV. A recombinant nucleic acid construct comprises a nucleic acid encoding a Cas9 and/or a guide RNA complementary to a target sequence in HIV as described herein, operably linked to a regulatory region suitable for expressing the Cas9 and/or a guide RNA complementary to a target sequence in HIV in the cell. It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for Cas9 can be modified such that optimal expression in a particular organism is obtained, using appropriate codon bias tables for that organism.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Yeast expression systems can also be used. For example, the non-fusion pYES2 vector (XbaI, SphI, ShoI, NotI, GstXI, EcoRI, BstXI, BamH1, SacI, Kpn1, and HindIII cloning sites; Invitrogen) or the fusion pYESHisA, B, C (XbaI, SphI, ShoI, NotI, BstXI, EcoRI, BamH1, SacI, KpnI, and HindIII cloning sites, N-terminal peptide purified with ProBond resin and cleaved with enterokinase; Invitrogen), to mention just two, can be employed according to the invention. A yeast two-hybrid expression system can also be prepared in accordance with the invention.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Suitable nucleic acid delivery systems include recombinant viral vector, typically sequence from at least one of an adenovirus, adenovirus-associated virus (AAV), helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, preferably about one polynucleotide. In some embodiments, the viral vector used in the invention methods has a pfu (plague forming units) of from about 10⁸ to about 5×10′ pfu. In embodiments in which the polynucleotide is to be administered with a non-viral vector, use of between from about 0.1 nanograms to about 4000 micrograms will often be useful e.g., about 1 nanogram to about 100 micrograms.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. The selection of appropriate promoters can readily be accomplished. An example of a suitable promoter is the 763-base-pair cytomegalovirus (CMV) promoter. Other suitable promoters which may be used for gene expression include, but are not limited to, the Rous sarcoma virus (RSV) (Davis, et al., Hum Gene Ther 4:151 (1993)), the SV40 early promoter region, the herpes thymidine kinase promoter, the regulatory sequences of the metallothionein (MMT) gene, prokaryotic expression vectors such as the β-lactamase promoter, the tac promoter, promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells, insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in myeloid cells, myelin basic protein gene control region which is active in oligodendrocyte cells in the brain, myosin light chain-2 gene control region which is active in skeletal muscle, and gonadotropic releasing hormone gene control region which is active in the hypothalamus. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety.

Delivery of vectors can also be mediated by exosomes. Exosomes are lipid nanovesicles released by many cell types. They mediate intercellular communication by transporting nucleic acids and proteins between cells. Exosomes contain RNAs, miRNAs, and proteins derived from the endocytic pathway. They may be taken up by target cells by endocytosis, fusion, or both. Typically, the receipt of endosomal contents alters the functions of the receiving cells (Lee, et al., 2012).

Exosomes can be harnessed to deliver nucleic acids to target cells. In a preferred method, exosomes are produced in vitro by producer cells, purified, and loaded with a nucleic acid cargo by electroporation, or by lipid transfection agents (Marcus and Leonard, 2013, Shtam, et al., 2013). The cargo can include expression constructs for a Cas endonuclease and one or more gRNAs. Suitable techniques can be found in Kooijmans, et al. (2012), Lee, et al. (2012), Marcus and Leonard (2013), Shtam, et al. (2013), or references therein. An exemplary kit for producing and loading exosomes is the ExoFect™ kit (System Biosciences, Inc., Mountain View, Calif.).

Exosomes can also be targeted for preferential uptake by particular cell types. A targeting strategy especially useful for the present invention is disclosed by Alvarez-Ervitti, et al. (2011). Using techniques disclosed therein, exosomes can be decorated with rabies viral glycoprotein (RVG) peptide. Exosomes bearing RVG home specifically to the brain, especially to neurons, oligodendrocytes, and microglia, with little nonspecific accumulation in other tissues.

The expression constructs of the present invention can also be delivered by means of nanoclews. Nanoclews are a cocoon-like DNA nanocomposites (Sun, et al., 2014). They can be loaded with nucleic acids for uptake by target cells and release in target cell cytoplasm. Methods for constructing nanoclews, loading them, and designing release molecules can be found in Sun, et al. (2014) and Sun, et al. (2015).

The gene editing constructs of the present invention can also be delivered not by induced expression by host cells, but by direct delivery, that is, delivery of a Cas nuclease protein, such as Cas9 protein, plus one of more gRNAs. Exosomes are a preferred vehicle for direct delivery, as they can be loaded with both proteins and RNAs (Alvarez-Ervitti, et al., 2011; Marcus and Leonard, 2013). An exemplary method of protein loading into exosomes is by the expression of a protein as a fusion with endosomal proteins such as lactadherin, in exosome producing cells. Another favorable feature of exosomes is their targetability to specific sites, such as the brain, as previously described. gRNAs can be loaded into the same exosomes as Cas nuclease protein, preferably, in the form of Cas/gRNA complexes. Cas endonucleases and gRNAs can alternatively be loaded into separate exosomes, for simultaneous or staged delivery.

Direct delivery of gene editing complexes can also be accomplished by menas of nanoclews. Sun, et al. (2015) disclose techniques for loading Cas9/gRNA complexes into nanoclews for uptake and release into receiving cells.

Direct delivery vehicles can be administered by any appropriate route, including, but not limited to, i.v., i.p, rectal, intrathecal, intracranial, inhalation, and per os, including in pill form.

Pharmaceutical Compositions

As described above, the compositions of the present invention can be prepared in a variety of ways known to one of ordinary skill in the art. Regardless of their original source or the manner in which they are obtained, the compositions of the invention can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having an HIV infection or at risk for contracting and HIV infection. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein in combination with one or more pharmaceutically acceptable carriers. We use the terms “pharmaceutically acceptable” (or “pharmacologically acceptable”) to refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the an isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI. Exosomes and nanoclews, as previously described, can also be utilized in pharmaceutical compositions according to the present invention.

The nucleic acids and vectors may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions may be formulated as a topical gel for blocking sexual transmission of HIV. The topical gel can be applied directly to the skin or mucous membranes of the male or female genital region prior to sexual activity. Alternatively or in addition the topical gel can be applied to the surface or contained within a male or female condom or diaphragm.

In some embodiments, the compositions can be formulated as a nanoparticle encapsulating a nucleic acid encoding Cas9 or a variant Cas9 and a guide RNA sequence complementary to a target HIV or vector comprising a nucleic acid encoding Cas9 and a guide RNA sequence complementary to a target HIV. Alternatively, the compositions can be formulated as a nanoparticle encapsulating a CRISPR-associated endonuclease polypeptide, e.g., Cas9 or a variant Cas9 and a guide RNA sequence complementary to a target.

The present formulations can encompass a vector encoding Cas9 and a guide RNA sequence complementary to a target HIV. The guide RNA sequence can include a sequence complementary to a single region, e.g. LTR A, B, C, or D or it can include any combination of sequences complementary to LTR A, B, C, and D. Alternatively the sequence encoding Cas9 and the sequence encoding the guide RNA sequence can be on separate vectors.

Methods of Treatment

The compositions disclosed herein are generally and variously useful for treatment of a subject having a retroviral infection, e.g., an HIV infection. We may refer to a subject, patient, or individual interchangeably. The methods are useful for targeting any HIV, for example, HIV-1, HIV-2, and any circulating recombinant form thereof. A subject is effectively treated whenever a clinically beneficial result ensues. This may mean, for example, a complete resolution of the symptoms of a disease, a decrease in the severity of the symptoms of the disease, or a slowing of the disease's progression. These methods can further include the steps of a) identifying a subject (e.g., a patient and, more specifically, a human patient) who has an HIV infection; and b) providing to the subject a composition comprising a nucleic acid encoding a CRISPR-associated nuclease, e.g., Cas9, and a guide RNA complementary to an HIV target sequence, e.g. an HIV LTR. A subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present invention, one can first determine whether a patient has a latent HIV-1 infection, and then make a determination as to whether or not to treat the patient with one or more of the compositions described herein. Monitoring can also be used to detect the onset of drug resistance and to rapidly distinguish responsive patients from nonresponsive patients. In some embodiments, the methods can further include the step of determining the nucleic acid sequence of the particular HIV harbored by the patient and then designing the guide RNA to be complementary to those particular sequences. For example, one can determine the nucleic acid sequence of a subject's LTR U3, R or U5 region and then design one or more guide RNAs to be precisely complementary to the patient's sequences.

The compositions are also useful for the treatment, for example, as a prophylactic treatment, of a subject at risk for having a retroviral infection, e.g., an HIV infection. These methods can further include the steps of a) identifying a subject at risk for having an HIV infection; b) providing to the subject a composition comprising a nucleic acid encoding a CRISPR-associated nuclease, e.g., Cas9, and a guide RNA complementary to an HIV target sequence, e.g. an HIV LTR. A subject at risk for having an HIV infection can be, for example, any sexually active individual engaging in unprotected sex, i.e., engaging in sexual activity without the use of a condom; a sexually active individual having another sexually transmitted infection; an intravenous drug user; or an uncircumcised man. A subject at risk for having an HIV infection can be, for example, an individual whose occupation may bring him or her into contact with HIV-infected populations, e.g., healthcare workers or first responders. A subject at risk for having an HIV infection can be, for example, an inmate in a correctional setting or a sex worker, that is, an individual who uses sexual activity for income employment or nonmonetary items such as food, drugs, or shelter.

The compositions can also be administered to a pregnant or lactating woman having an HIV infection in order to reduce the likelihood of transmission of HIV from the mother to her offspring. A pregnant woman infected with HIV can pass the virus to her offspring transplacentally in utero, at the time of delivery through the birth canal or following delivery, through breast milk. The compositions disclosed herein can be administered to the HIV infected mother either prenatally, perinatally or postnatally during the breast-feeding period, or any combination of prenatal, perinatal, and postnatal administration. Compositions can be administered to the mother along with standard antiretroviral therapies as described below. In some embodiments, the compositions of the invention are also administered to the infant immediately following delivery and, in some embodiments, at intervals thereafter. The infant also can receive standard antiretroviral therapy.

The methods and compositions disclosed herein are useful for the treatment of retroviral infections. Exemplary retroviruses include human immunodeficiency viruses, e.g. HIV-1, HIV-2; simian immunodeficiency virus (SIV); feline immunodeficiency virus (FIV); bovine immunodeficiency virus (BIV); equine infectious anemia virus (EIAV); and caprine arthritis/encephalitis virus (CAEV). The methods disclosed herein can be applied to a wide range of species, e.g., humans, non-human primates (e.g., monkeys), horses or other livestock, dogs, cats, ferrets or other mammals kept as pets, rats, mice, or other laboratory animals.

The methods of the invention can be expressed in terms of the preparation of a medicament. Accordingly, the invention encompasses the use of the agents and compositions described herein in the preparation of a medicament. The compounds described herein are useful in therapeutic compositions and regimens or for the manufacture of a medicament for use in treatment of diseases or conditions as described herein.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

The compositions may also be administered with another therapeutic agent, for example, an anti-retroviral agent, used in HAART. Exemplary antiretroviral agents include reverse transcriptase inhibitors (e.g., nucleoside/nucleotide reverse transcriptase inhibitors, zidovudine, emtricitibine, lamivudine and tenofivir; and non-nucleoside reverse transcriptase inhibitors such as efavarenz, nevirapine, rilpivirine); protease inhibitors, e.g., tipiravir, darunavir, indinavir; entry inhibitors, e.g., maraviroc; fusion inhibitors, e.g., enfuviritide; or integrase inhibitors e.g., raltegrivir, dolutegravir. Exemplary antiretroviral agents can also include multi-class combination agents for example, combinations of emtricitabine, efavarenz, and tenofivir; combinations of emtricitabine; rilpivirine, and tenofivir; or combinations of elvitegravir, cobicistat, emtricitabine and tenofivir.

Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks. The therapeutic agents may be administered under a metronomic regimen, e.g., continuous low-doses of a therapeutic agent.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. The liposomes will be targeted to and taken up selectively by the organ.

Also provided, are methods of inactivating a retrovirus, for example a lentivirus such as a human immunodeficiency virus, a simian immunodeficiency virus, a feline immunodeficiency virus, or a bovine immunodeficiency virus in a mammalian cell. The human immunodeficiency virus can be HIV-1 or HIV-2. The human immunodeficiency virus can be a chromosomally integrated provirus. The mammalian cell can be any cell type infected by HIV, including, but not limited to CD4+ lymphocytes, macrophages, fibroblasts, monocytes, T lymphocytes, B lymphocytes, natural killer cells, dendritic cells such as Langerhans cells and follicular dendritic cells, hematopoietic stem cells, endothelial cells, brain microglial cells, and gastrointestinal epithelial cells. Such cell types include those cell types that are typically infected during a primary infection, for example, a CD4+ lymphocyte, a macrophage, or a Langerhans cell, as well as those cell types that make up latent HIV reservoirs, i.e., a latently infected cell.

The methods can include exposing the cell to a composition comprising an isolated nucleic acid encoding a gene editing complex comprising a CRISPR-associated endonuclease and one or more guide RNAs wherein the guide RNA is complementary to a target nucleic acid sequence in the retrovirus. In a preferred embodiment, as previously described, the method of inactivating a proviral DNA integrated into the genome of a host cell latently infected with a retrovirus includes the steps of treating the host cell with a composition comprising a CRISPR-associated endonuclease, and two or more different guide RNAs (gRNAs), wherein each of the at least two gRNAs is complementary to a different target nucleic acid sequence in the proviral DNA; and inactivating the proviral DNA. The at least two gRNAs can be configured as a single sequence or as a combination of one or more different sequences, e.g., a multiplex configuration. Multiplex configurations can include combinations of two, three, four, five, six, seven, eight, nine, ten, or more different gRNAs, for example any combination of sequences in U3, R, or U5. In some embodiments, combinations of LTR A, LTR B, LTR C and LTR D can be used. In some embodiments, combinations of any of the sequences LTR A (SEQ ID NO: 96), LTR B (SEQ ID NO: 121), LTR C (SEQ ID NO: 87), and LTR D (SEQ ID NO: 110), can be used. In experiments described in the Examples, the use of two different gRNAs caused the excision of the viral sequences between the cleavage sites recognized by the CRISPR endonuclease. The excised region can include the entire HIV-1 genome. The treating step can take place in vivo, that is, the compositions can be administered directly to a subject having HIV infection. The methods are not so limited however, and the treating step can take place ex vivo. For example, a cell or plurality of cells, or a tissue explant, can be removed from a subject having an HIV infection and placed in culture, and then treated with a composition comprising a CRISPR-associated endonuclease and a guide RNA wherein the guide RNA is complementary to the nucleic acid sequence in the human immunodeficiency virus. As described above, the composition can be a nucleic acid encoding a CRISPR-associated endonuclease and a guide RNA wherein the guide RNA is complementary to the nucleic acid sequence in the human immunodeficiency virus; an expression vector comprising the nucleic acid sequence; or a pharmaceutical composition comprising a nucleic acid encoding a CRISPR-associated endonuclease and a guide RNA wherein the guide RNA is complementary to the nucleic acid sequence in the human immunodeficiency virus; or an expression vector comprising the nucleic acid sequence. In some embodiments, the gene editing complex can comprise a CRISPR-associated endonuclease polypeptide and a guide RNA wherein the guide RNA is complementary to the nucleic acid sequence in the human immunodeficiency virus.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

Standard methods, for example, immunoassays to detect the CRISPR-associated endonuclease, or nucleic acid-based assays such as PCR to detect the gRNA, can be used to confirm that the complex has been taken up and expressed by the cell into which it has been introduced. The engineered cells can then be reintroduced into the subject from whom they were derived as described below.

The gene editing complex comprises a CRISPR-associated nuclease, e.g., Cas9, and a guide RNA complementary to the retroviral target sequence, for example, an HIV target sequence. The gene editing complex can introduce various mutations into the proviral DNA. The mechanism by which such mutations inactivate the virus can vary, for example the mutation can affect proviral replication, viral gene expression or proviral excision. The mutations may be located in regulatory sequences or structural gene sequences and result in defective production of HIV. The mutation can comprise a deletion. The size of the deletion can vary from a single nucleotide base pair to about 10,000 base pairs. In some embodiments, the deletion can include all or substantially all of the proviral sequence. In some embodiments the deletion can include the entire proviral sequence. The mutation can comprise an insertion; that is the addition of one or more nucleotide base pairs to the pro-viral sequence. The size of the inserted sequence also may vary, for example from about one base pair to about 300 nucleotide base pairs. The mutation can comprise a point mutation, that is, the replacement of a single nucleotide with another nucleotide. Useful point mutations are those that have functional consequences, for example, mutations that result in the conversion of an amino acid codon into a termination codon or that result in the production of a nonfunctional protein.

In exemplary multiplex methods for inactivating proviral DNA integrated into the genome of a host cell, as demonstrated in Examples 2-5, two different gRNA sequences are deployed, with each gRNA sequence targeting a different site in the proviral DNA. That is, the methods include the steps of exposing the host cell to a composition including an isolated nucleic acid encoding a CRISPR-associated endonuclease; an isolated nucleic acid sequence encoding a first gRNA having a first spacer sequence that is complementary to a first target protospacer sequence in a proviral DNA; and an isolated nucleic acid encoding a second gRNA having a second spacer sequence that is complementary to a second target protospacer sequence in the proviral DNA; expressing in the host cell the CRISPR-associated endonuclease, the first gRNA, and the second gRNA; assembling, in the host cell, a first gene editing complex including the CRISPR-associated endonuclease and the first gRNA; and a second gene editing complex including the CRISPR-associated endonuclease and the second gRNA; directing the first gene editing complex to the first target protospacer sequence by complementary base pairing between the first spacer sequence and the first target protospacer sequence; directing the second gene editing complex to the second target protospacer sequence by complementary base pairing between the second spacer sequence and the second target protospacer sequence; cleaving the proviral DNA at the first target protospacer sequence with the CRISPR-associated endonuclease; cleaving the proviral DNA at the second target protospacer sequence with the CRISPR-associated endonuclease; and inducing at least one mutation in the proviral DNA. The same multiplex method is readily incorporated into methods for treating a subject having a human immunodeficiency virus, and for reducing the risk of a human immunodeficiency virus infection. It will be understood that the term “composition” can include not only a mixture of components, but also separate components that are not necessarily administered simultaneously. As a non-limiting example, a composition according to the present invention can include separate component preparations of nucleic acid sequences encoding a Cas9 nuclease, a first gRNA, and a second gRNA, with each component being administered sequentially in an infusion, during a time frame that results in a host cell being exposed to all three components.

In other embodiments, the compositions comprise a cell which has been transformed or transfected with one or more Cas/gRNA vectors. In some embodiments, the methods of the invention can be applied ex vivo. That is, a subject's cells can be removed from the body and treated with the compositions in culture to excise HIV sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The gRNA expression cassette can be easily delivered to a subject by methods known in the art, for example, methods which deliver siRNA. In some aspects, the Cas may be a fragment wherein the active domains of the Cas molecule are included, thereby cutting down on the size of the molecule. Thus, the, Cas9/gRNA molecules can be used clinically, similar to the approaches taken by current gene therapy. In particular, a Cas9/multiplex gRNA stable expression stem cell or iPS cells for cell transplantation therapy as well as HIV-1 vaccination will be developed for use in subjects.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

Articles of Manufacture

The compositions described herein can be packaged in suitable containers labeled, for example, for use as a therapy to treat a subject having a retroviral infection, for example, an HIV infection or a subject at for contracting a retroviral infection, for example, an HIV infection. The containers can include a composition comprising a nucleic acid sequence encoding a CRISPR-associated endonuclease, for example, a Cas9 endonuclease, and a guide RNA complementary to a target sequence in a human immunodeficiency virus, or a vector encoding that nucleic acid, and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including at least one composition of the invention, e.g., a nucleic acid sequence encoding a CRISPR-associated endonuclease, for example, a Cas9 endonuclease, and a guide RNA complementary to a target sequence in a human immunodeficiency virus, or a vector encoding that nucleic acid and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

In some embodiments, the kits can include one or more additional antiretroviral agents, for example, a reverse transcriptase inhibitor, a protease inhibitor or an entry inhibitor. The additional agents can be packaged together in the same container as a nucleic acid sequence encoding a CRISPR-associated endonuclease, for example, a Cas9 endonuclease, and a guide RNA complementary to a target sequence in a human immunodeficiency virus, or a vector encoding that nucleic acid or they can be packaged separately. The nucleic acid sequence encoding a CRISPR-associated endonuclease, for example, a Cas9 endonuclease, and a guide RNA complementary to a target sequence in a human immunodeficiency virus, or a vector encoding that nucleic acid and the additional agent may be combined just before use or administered separately.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

### Example 1: Materials and Methods

Plasmid Preparation:

Vectors containing human Cas9 and gRNA expression cassette, pX260, and pX330 (Addgene) were utilized to create various constructs, LTR-A, B, C, and D.

Cell Culture and Stable Cell Lines:

TZM-b1 reporter and U1 cell lines were obtained from the NIH AIDS Reagent Program and CHME5 microglial cells are known in the art.

Immunohistochemistry and Western Blot:

Standard methods for immunocytochemical observation of the cells and evaluation of protein expression by Western blot were utilized.

Firefly-Luciferase Assay:

Cells were lysed 24 h post-treatment using Passive Lysis Buffer (Promega) and assayed with a Luciferase Reporter Gene Assay kit (Promega) according to the manufacturer's protocol. Luciferase activity was normalized to the number of cells determined by a parallel MTT assay (Vybrant, Invitrogen)

p24 ELISA:

After infection or reactivation, the levels of HIV-1 viral load in the supernatants were quantified by p24 Gag ELISA (Advanced BioScience Laboratories, Inc) following the manufacturer's protocol. To assess cell viability upon treatments, MTT assay was performed in parallel according to the manufacturer's manual (Vybrant, Invitrogen).

EGFP Flow Cytometry:

Cells were trypsinized, washed with PBS and fixed in 2% paraformaldehyde for 10 min at room temperature, then washed twice with PBS and analyzed using a Guava EasyCyte Mini flow cytometer (Guava Technologies).

HIV-1 Reporter Virus Preparation and Infections:

HEK293T cells were transfected using Lipofectamine 2000 reagent (Invitrogen) with pNL4-3-ΔE-EGFP (NIH AIDS Research and Reference Reagent Program). After 48 h, the supernatant was collected, 0.45 μm filtered and tittered in HeLa cells using EGFP as an infection marker. For viral infection, stable Cas9/gRNA TZM-bI cells were incubated 2 h with diluted viral stock, and then washed twice with PBS. At 2 and 4 d post-infection, cells were collected, fixed and analyzed by flow cytometry for EGFP expression, or genomic DNA purification was performed for PCR and whole genome sequencing.

Genomic DNA Amplification, PCR, TA-Cloning, and Sanger Sequencing, GenomeWalker Link PCR:

Standard methods for DNA manipulation for cloning and sequencing were utilized. For identification of the integration sites of HIV-1, we utilized Lenti-X™ integration site analysis kit was used.

Surveyor Assay:

The presence of mutations in PCR products was examined using a SURVEYOR Mutation Detection Kit (Transgenomic) according to the protocol from the manufacturer. Briefly heterogeneous PCR product was denatured for 10 min in 95° C. and hybridized by gradual cooling using a thermocycler. Next, 300 ng of hybridized DNA (9 μl) was subjected to digestion with 0.25 μl of SURVEYOR Nuclease in the presence of 0.25 μl SURVEYOR Enhancer S and 15 mM MgCl₂ for 4 h at 42° C. Then Stop Solution was added and samples were resolved in 2% agarose gel together with equal amounts of undigested PCR product controls.

Some PCR products were used for restriction fragment length polymorphism analysis. Equal amounts of the PCR products were digested with BsaJI. Digested DNA was separated on an ethidium bromide-contained agarose gel (2%). For sequencing, PCR products were cloned using a TA Cloning® Kit Dual Promoter with pCR™II vector (Invitrogen). The insert was confirmed by digestion with EcoRI and positive clones were sent to Genewiz for Sanger sequencing.

Selection of LTR Target Sites, Whole Genome Sequencing and Bioinformatics and Statistical Analysis.

We utilized Jack Lin's CRISPR/Cas9 gRNA finder tool for initial identification of potential target sites within the LTR.

Plasmid Preparation.

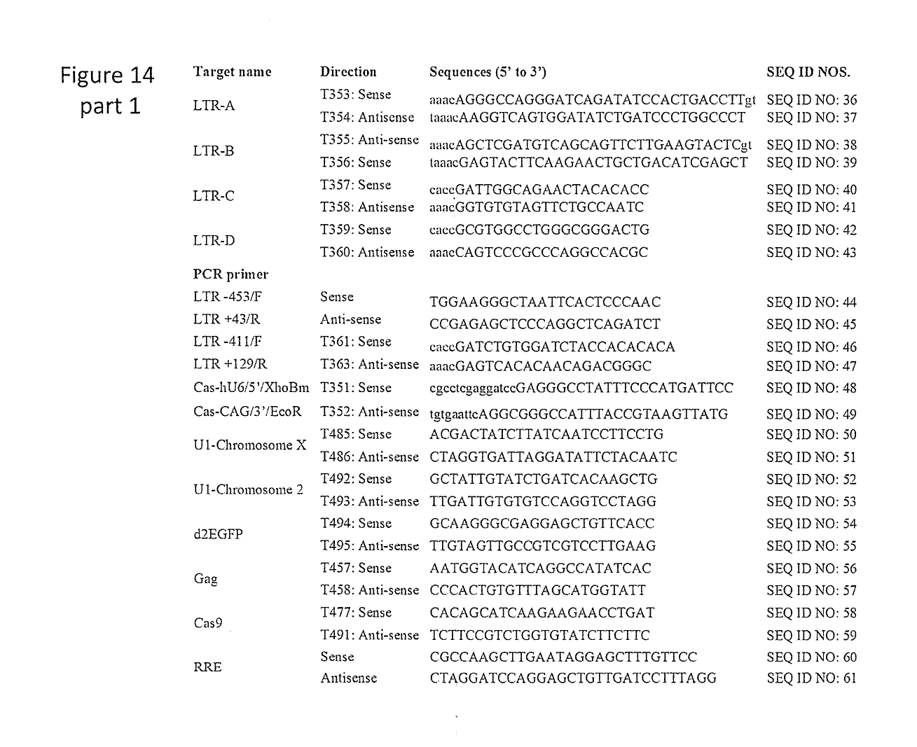

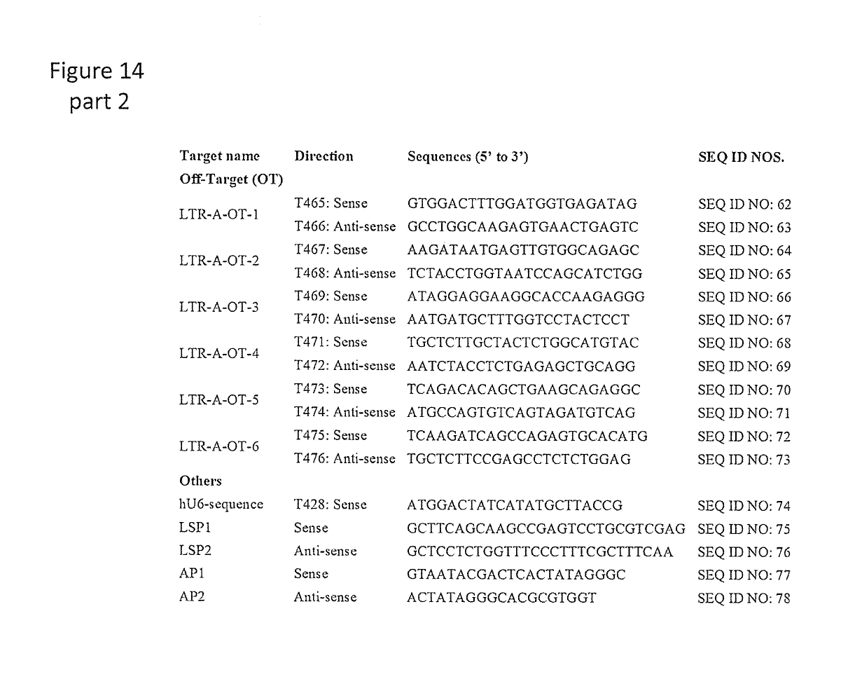

DNA segment expressing LTR-A or LTR-B for pre-crRNA was cloned into the pX260 vector that contains the puromycin selection gene (Addgene, plasmid #42229). DNA segments expressing LTR-C or LTR-D for the chimeric crRNA-tracrRNA were cloned into the pX330 vector (Addgene, plasmid #42230). Both vectors contain a humanized Cas9 coding sequence driven by a CAG promoter and a gRNA expression cassette driven by a human U6 promoter. The vectors were digested with BbsI and treated with Antarctic Phosphatase, and the linearized vector was purified with a Quick nucleotide removal kit (Qiagen). A pair of oligonucleotides for each targeting site (FIG. 14, Alpha DNA) was annealed, phosphorylated, and ligated to the linearized vector. The gRNA expression cassette was sequenced with U6 sequencing primer (FIG. 14) in GENEWIZ. For pX330 vectors, we designed a pair of universal PCR primers with overhang digestion sites (FIG. 14) that can tease out the gRNA expression cassette (U6-gRNA-crRNA-stem-tracrRNA) for direct transfection or subcloning to other vectors.

Cell Culture.

TZM-bI reporter cell line from Dr John C. Kappes, Dr Xiaoyun Wu and Tranzyme Inc, U1/Hiv-1 cell line from Dr. Thomas Folks and J-Lat full length clone from Dr. Eric Verdin were obtained through the NIH AIDS Reagent Program, Division of AIDS, NIAID, NIH. CHME5/HIV fetal microglia cell line were generated as previously described. TZM-bI and CHME5 cells were cultured in Dulbecco's minimal essential medium high glucose supplemented with 10% heat-inactivated fetal bovine serum (FBS) and 1% penicillin/streptomycin. U1 and J-Lat cells were cultured in RPMI 1640 containing 2.0 mM L-glutamine, 10% FBS and 1% penicillin/streptomycin.

Stable Cell Lines and Subcloning.

TZM-bI or CHME5/HIV cells were seeded in 6-well plates at 1.5×10⁵ cells/well and transfected using Lipofectamine 2000 reagent (Invitrogen) with 1 μg of pX260 (for LTR-A and B) or 1 μg/0.1 μg of pX330/pX260 (for LTR-C and D) plasmids. Next day, cells were transferred into 100-mm dishes and incubated with growth medium containing 1 μg/ml of puromycin (Sigma). Two weeks later, surviving cell colonies were isolated using cloning cylinders (Corning). U1 cells (1.5×10⁵) were electroporated with 1 μg of DNA using 10 μl tip, 3×10 ms 1400 V impulses at The Neon™ Transfection System (Invitrogen). Cells were selected with 0.5 μg/ml of puromycin for two weeks. The stable clones were subcultured using a limited dilution method in 96-well plates and single cell-derived subclones were maintained for further studies.

Immunocytochemistry and Western Blot.

The Cas9/gRNA stable expression TZM-bI cells were cultured in 8-well chamber slides for 2 days and fixed for 10 min in 4% paraformaldehyde/PBS. After three rinses, the cells were treated with 0.5% Triton X-100/PBS for 20 min and blocked in 10% donkey serum for 1 h. Cells were incubated overnight at 4° C. with mouse anti-Flag M2 primary antibody (1:500, Sigma). After rinsing three times, cells were incubated for 1 h with donkey anti-mouse Alexa-Fluor-594 secondary antibodies, and incubated with Hoechst 33258 for 5 min. After three rinses with PBS, the cells were coverslipped with anti-fading aqueous mounting media (Biomeda) and analyzed under a Leica DMI6000B fluorescence microscope.

TZM-bI cells cultured in 6-well plate were solubilized in 200 μl of Triton X-100-based lysis buffer containing 20 mM Tris-HCl (pH 7.4), 1% Triton X-100, 5 mM ethylenediaminetetraacetic acid, 5 mM dithiothreitol, 150 mM NaCl, 1 mM phenylmethylsulfonyl fluoride, 1× nuclear extraction proteinase inhibitor cocktail (Cayman Chemical, Ann Arbor, Mich.), 1 mM sodium orthovanadate and 30 mM NaF. Cell lysates were rotated at 4° C. for 30 min. Nuclear and cellular debris was cleared by centrifugation at 20,000 g for 20 min at 4° C. Equal amounts of lysate proteins (20 μg) were denatured by boiling for 5 min in sodium dodecyl sulphate (SDS) sample buffer, fractionated by SDS-polyacrylamide gel electrophoresis in tris-glycine buffer, and transferred to nitrocellulose membrane (BioRad). The SeeBlue prestained standards (Invitrogen) were used as a molecular weight reference. Blots were blocked in 5% BSA/tris-buffered saline (pH 7.6) plus 0.1% Tween-20 (TBS-T) for 1 h and then incubated overnight at 4° C. with mouse anti-Flag M2 monoclonal antibody (1:1000, Sigma) or mouse anti-GAPDH monoclonal antibody (1:3000, Santa Cruz Biotechnology). After washing with TBS-T, the blots were incubated with IRDye 680LT-conjugated anti-mouse antibody for 1 h at room temperature. Membranes were scanned and analyzed using an Odyssey Infrared Imaging System (LI-COR Biosciences).

Firefly-Luciferase Assay.

Cells were lysed 24 h post-treatment using Passive Lysis Buffer (Promega) and assayed with a Luciferase Reporter Gene Assay kit (Promega) according to the protocol of the manufacturer. Luciferase activity was normalized to the number of cells determined by parallel MTT assay (Vybrant, Invitrogen).

p24 ELISA

After infection or reactivation, the HIV-1 viral load levels in the supernatants were quantified by p24 Gag ELISA (Advanced BioScience Laboratories, Inc) following the manufacturer's protocol. To assess the cell viability upon treatments, MTT assay was performed in parallel according to the manufacturer's protocol (Vybrant, Invitrogen).

EGFP Flow Cytometry.

Cells were trypsinized, washed with PBS and fixed in 2% paraformaldehyde for 10 min at room temperature, then washed twice with PBS and analyzed using a Guava EasyCyte Mini flow cytometer (Guava Technologies).

Hiv-1 Reporter Virus Preparation and Infections.

HEK293T cells were transfected using Lipofectamine 2000 reagent (Invitrogen) with pNL4-3-ΔE-EGFP, SF162 and JRFL (NIH AIDS Research and Reference Reagent Program). For pseudotyped pNL4-3-ΔE-EGFP, the VSVG vector was cotransfected. After 48 h, the supernatant was collected, 0.45 μm filtered and tittered in HeLa cells using expressed EGFP as an infection marker. For viral infection, stable Cas9/gRNATZM-bI cells were incubated 2 h with a diluted viral stock, and washed twice with PBS. At 2 and 4 days post-infection, cells were collected, fixed and analyzed by flow cytometry for EGFP expression, or genomic DNA purification was performed for PCR and whole genome sequencing.

Genomic DNA Purification, PCR, TA-Cloning and Sanger Sequencing.

Genomic DNA was isolated from cells using an ArchivePure DNA cell/tissue purification kit (5PRIME) according to the protocol recommended by the manufacturer. One hundred ng of extracted DNA were subjected to PCR using a high-fidelity FailSafe PCR kit (Epicentre) using primers listed in FIG. 14. Three steps of standard PCR were carried out for 30 cycles with 55° C. annealing and 72° C. extension. The products were resolved in 2% agarose gel. The bands of interest were gel-purified and cloned into pCRII T-A vector (Invitrogen), and the nucleotide sequence of individual clones was determined by sequencing at Genewiz using universal T7 and/or SP6 primers.

Conventional and Real-Time Reverse Transcription (RT)-PCR.

For total RNA extraction, cells were processed with an RNeasy Mini kit (Qiagen) as per manufacturer's instructions. The potentially residual genomic DNA was removed through on-column DNase digestion with an RNase-Free DNase Set (Qiagen). One μg of RNA for each sample was reversely transcribed into cDNAs using random hexanucleotide primers with a High Capacity cDNA Reverse Transcription Kit (Invitrogen, Grand Island, N.Y.). Conventional PCR was performed using a standard protocol. Quantitative PCR (qPCR) analyses were carried out in a LightCycler480 (Roche) using an SYBR® Green PCR Master Mix Kit (Applied Biosystems). The RT reactions were diluted to 5 ng of total RNA per micro-liter of reactions and 2 μl was used in a 20-μl PCR reaction. For qPCR analysis of HIV-1 proviruses, 50 ng of genomic DNA were used. The primers were synthesized in AlphaDNA and shown in FIG. 14. The primers for human housekeeping genes GAPDH and RPL13A were obtained from RealTimePrimers (Elkins Park, Pa.). Each sample was tested in triplicate. Cycle threshold (Ct) values were obtained graphically for the target genes and house-keeping genes. The difference in Ct values between the housekeeping gene and target gene was represented as ΔCt values. The ΔΔCt values were obtained by subtracting the ΔCt values of control samples from those of experimental samples. Relative fold or percentage change was calculated as 2-ΔΔCt. In some cases, absolute quantification was performed using the pNL4-3-ΔE-EGFP plasmid spiked in human genomic DNA as a standard. The number of HIV-1 viral copies was calculated based on standard curve after normalization with housekeeping gene.

GenomeWalker Link PCR and Long-Range PCR.

The integration sites of HIV-1 in host cells were identified using a Lenti-X™ Integration Site Analysis kit (Clontech) following the manufacturer's instruction. Briefly, high quality genomic DNAs were extracted from U1 cells using a NucleoSpin Tissue kit (Clontech). To construct the viral integration libraries, each genomic DNA sample was digested with blunt-end-generating digestion enzymes Dra I, Ssp I or HpaI separately overnight at 37° C. The digestion efficiency was verified by electrophoresis on 0.6% agarose. The digested DNA was purified using a NucleoSpin Gel and PCR Clean-Up kit followed by ligation of the digested genomic DNA fragments to GenomeWalker™ Adaptor at 16° C. overnight. The ligation reaction was stopped by incubation at 70° C. for 5 min and diluted 5 times with TE buffer. The primary PCR was performed on the DNA segments with adaptor primer 1 (AP1) and LTR-specific primer 1 (LSP1) using Advantage 2 Polymerase Mix followed by a secondary (nested) PCR using AP2 and LSP2 primers (FIG. 14). The secondary PCR products were separated on 1.5% ethidium bromide-containing agarose gel. The major bands were gel-purified and cloned into pCRII T-A vector (Invitrogen), and the nucleotide sequence of individual clones was determined by sequencing at Genewiz using universal T7 and SP6 primers. The sequence reads were analyzed by NCBI BLAST searching. Two integration sites of HIV-1 in U1 cells were identified in chromosomes X and 2. A pair of primers covering each integration site (FIG. 14) was synthesized in AlphaDNA. Long-range PCR using the U1 genomic DNA was performed with a Phusion High-Fidelity PCR kit (New England Biolabs) following the manufacturer's protocol. The PCR products were visualized on 1% agarose gel and validated by Sanger sequencing.

Surveyor Assay.

The presence of mutations in PCR products was tested using a SURVEYOR Mutation Detection Kit (Transgenomic) according to the protocol of the manufacturer. Briefly heterogeneous PCR products were denatured for 10 min in 95° C. and hybridized by gradual cooling using a thermocycler. Next 300 ng of hybridized DNA (9 ul) was subjected to digestion with 0.25 μl of SURVEYOR Nuclease in the presence of 0.25 μl SURVEYOR Enhancer S and 15 mM MgCl₂ for 4h at 42° C. Then Stop Solution was added and samples were resolved in 2% agarose gel together with equal amounts of undigested PCR products.

Some PCR products were used for restriction fragment length polymorphism analysis. Equal amount of PCR products were digested with BsaJI. Digested DNA was separated on an ethidium bromide-contained agarose gel (2%). For sequencing, PCR products were cloned using a TA Cloning® Kit Dual Promoter with pCR™II vector (Invitrogen). The insert was confirmed by digestion with EcoRI and positive clones were sent to Genwiz for Sanger sequencing.

Selection of LTR Target Sites and Prediction of Potential Off-Target Sites.

For initial studies, we obtained the LTR promoter sequence (−411 to −10) of the integrated lentiviral LTR-luciferase reporter by TA-cloning sequencing of PCR products from the genome of human TZM-bI cells because of potential mutation of LTR during passaging. This promoter sequence has 100% match to the 5′-LTR of pHR′-CMV-LacZ lentiviral vector (AF105229). Thus, sense and antisense sequences of the full-length pHR′ 5′-LTR (634 bp) were utilized to search for Cas9/gRNA target sites containing 20 bp gRNA targeting sequence plus the PAM sequence (NRG) using Jack Lin's CRISPR/Cas9 gRNA finder tool. The number of potential off-targets with exact match was predicted by blasting each gRNA targeting sequence plus NRG (AGG, TGG, GGG and CGG; AAG, TAG, GAG, CAG) against all available human genomic and transcript sequences using the NCBI/blastn suite with E-value cutoff 1,000 and word size 7. After pressing Control+F, copy/paste the target sequence (1-23 through 9-23 nucleotides) and find the number of genomic targets with 100% match to the target sequence. The number of off-targets for each search was divided by 3 because of repeated genome library.

Whole Genome Sequencing and Bioinformatics Analysis.

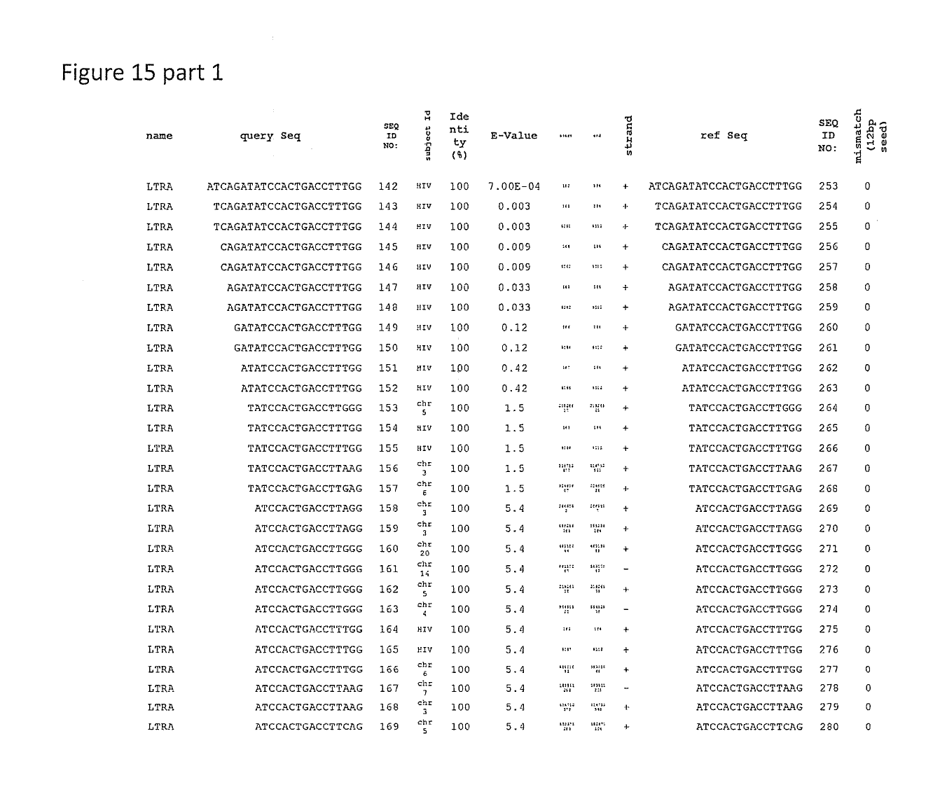

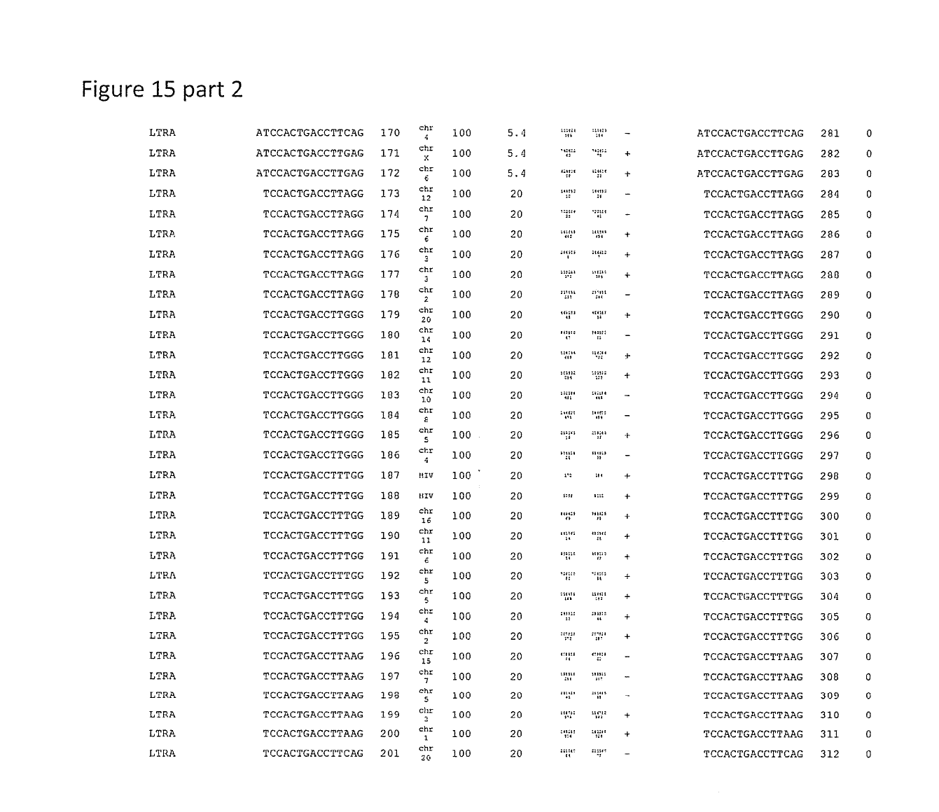

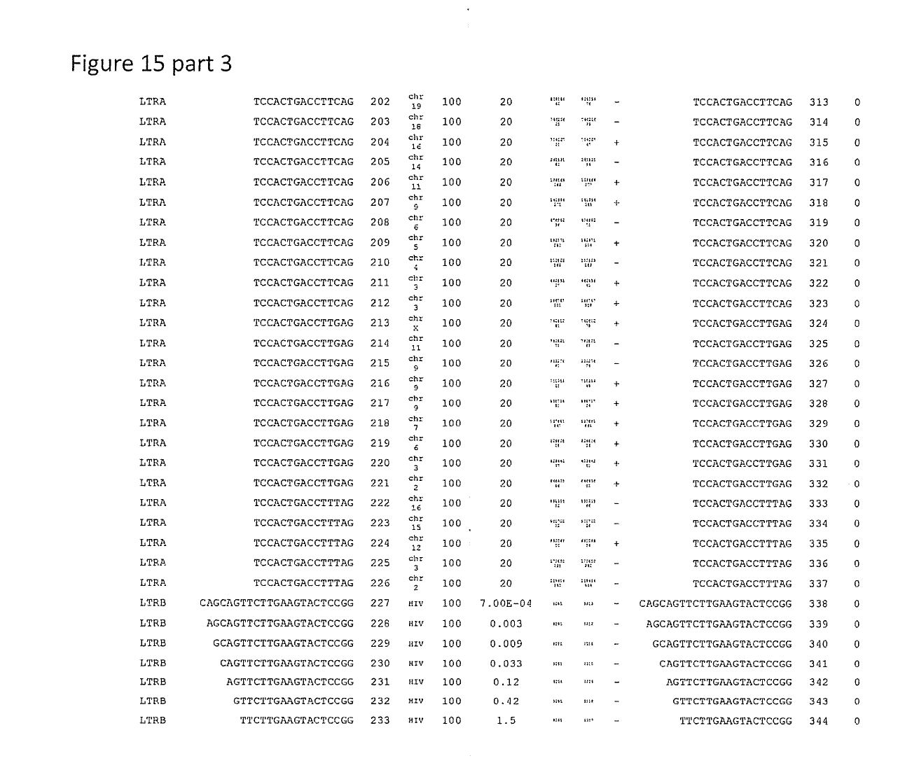

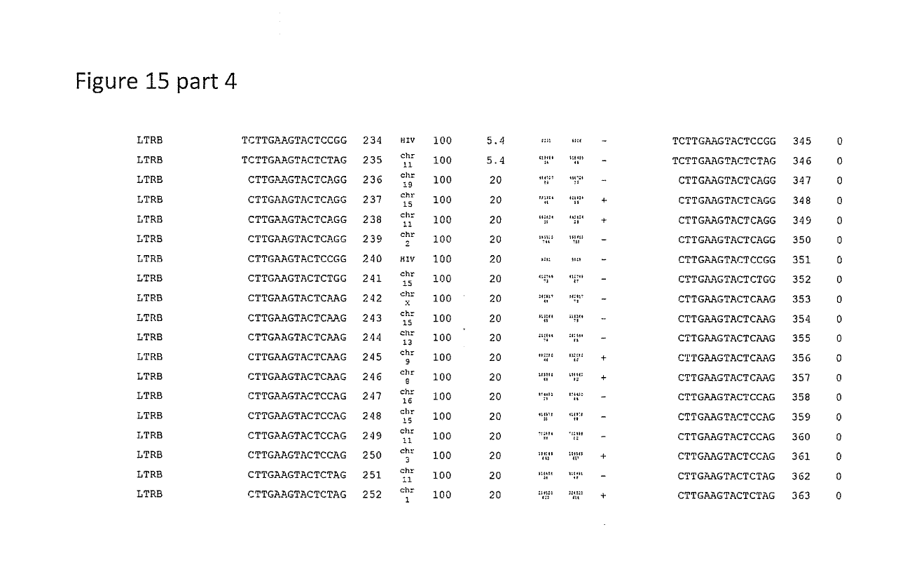

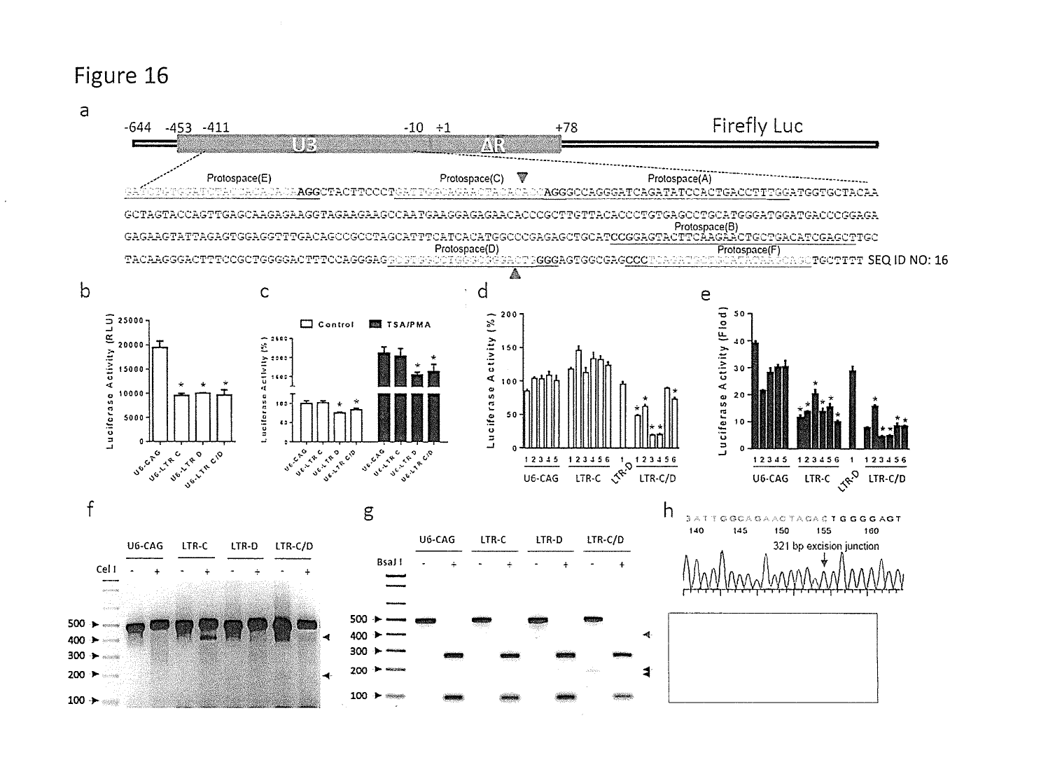

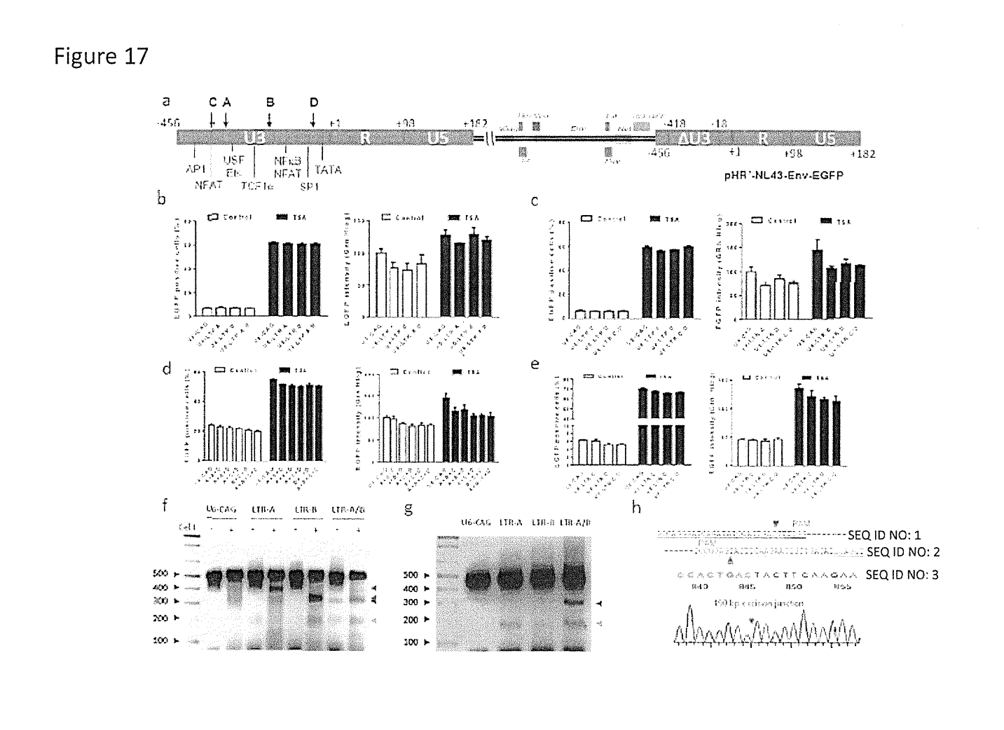

The control subclone C1 and experimental subclone AB7 of TZM-bI cells were validated for target cut efficiency and functional suppression of the LTR-luciferase reporter. The genomic DNA was isolated with NucleoSpin Tissue kit (Clontech). The DNA samples were submitted to the NextGen sequencing facility at Temple University Fox Chase Cancer Center. Duplicated genomic DNA libraries were prepared from each subclone using a NEBNext Ultra DNA Library Prep Kit for Illumina (New England Biolab) following the manufacturer's instruction. All libraries were sequenced with paired-end 141-bp reads in two Illumina Rapid Run flowcells on HiSeq 2500 instrument (Illumina). Demultiplexed read data from the sequenced libraries were sent to AccuraScience, LLC for professional bioinformatics analysis. Briefly, the raw reads were mapped against human genome (hg19) and HIV-1 genome by using Bowtie2. A genomic analysis toolkit (GATK, version 2.8.1) was used for the duplicated read removal, local alignment, base quality recalibration and indel calling. The confidence scores 10 and 30 were the thresholds for low quality (LowQual) and high confidence calling (PASS). The potential off-target sites of LTR-A and LTR-B with various mismatches were predicted by NCBI/blastn suite as described above and by a CRISPR Design Tool. All the potential gRNA target sites (FIG. 15) were used to map the ±300 bp regions around each indel identified by GATK. The locations of the overlapped regions in the human genome and HIV-1 genome were compared between the control C1 and experimental AB7.

Statistical Analysis.

The quantitative data represented mean±standard deviation from 3-5 independent experiments, and were evaluated by Student's t-test or ANOVA and Newman-Keuls multiple comparison test. A p value that is <0.05 or 0.01 was considered as a statistically significant difference.

### Example 2: Cas9/LTR-gRNA Suppresses HIV-1 Reporter Virus Production in CHME5 Microglial Cells Latently Infected with HIV-1

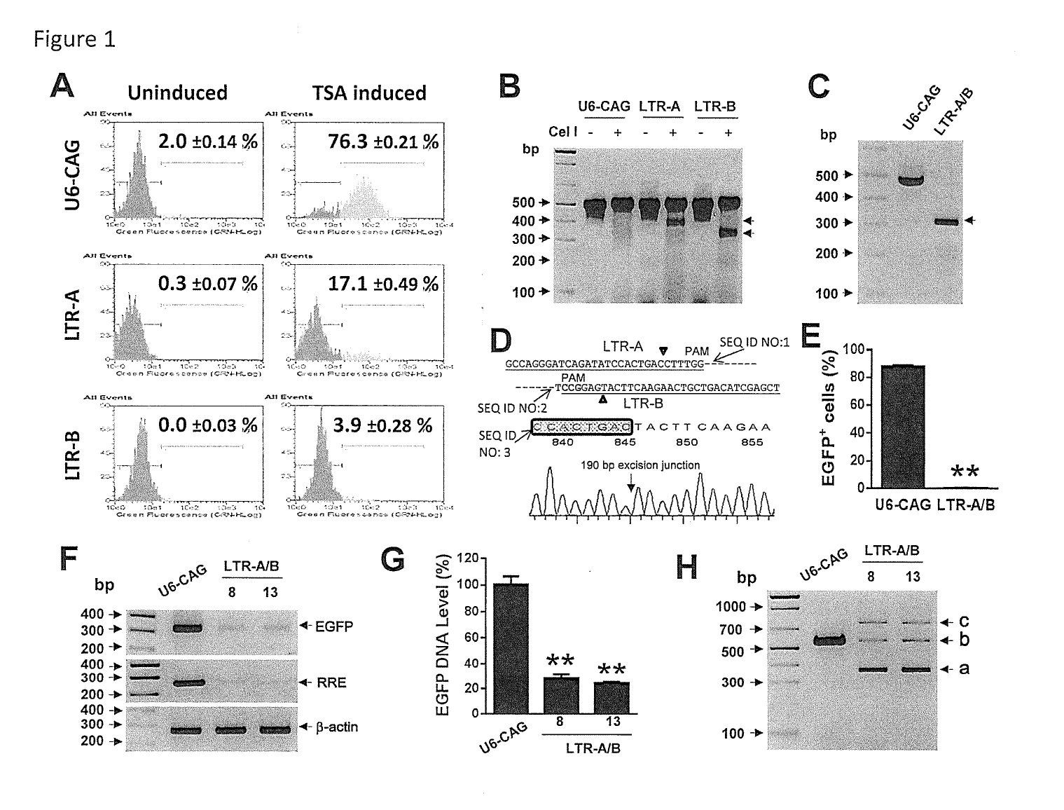

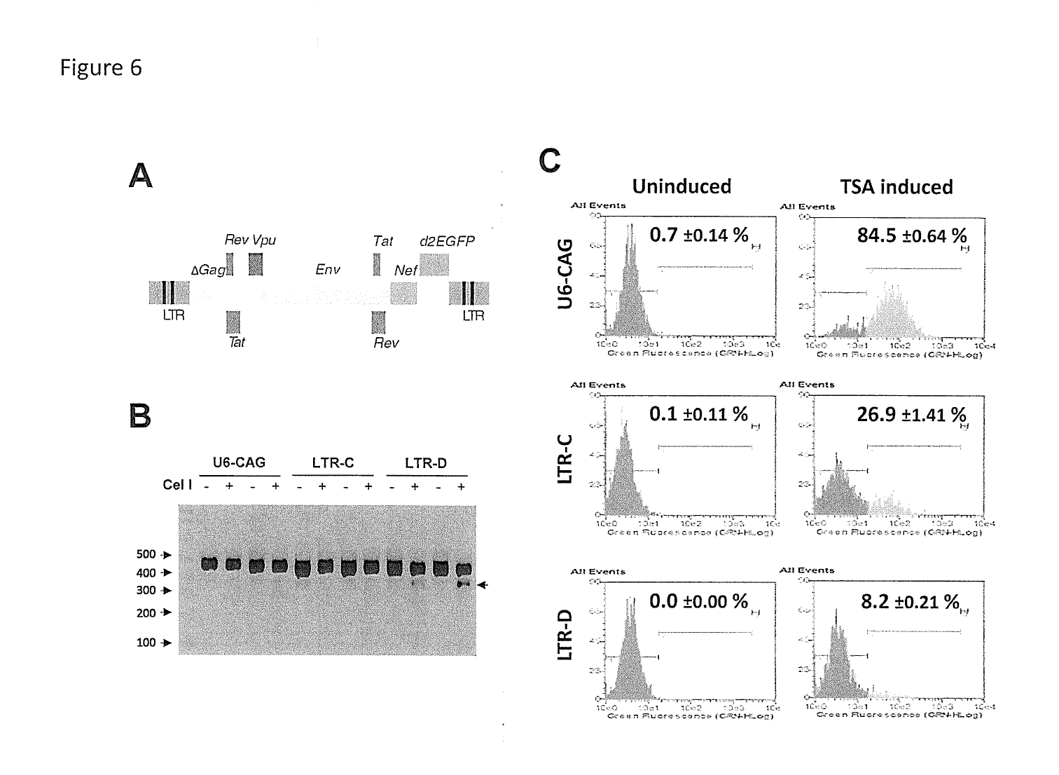

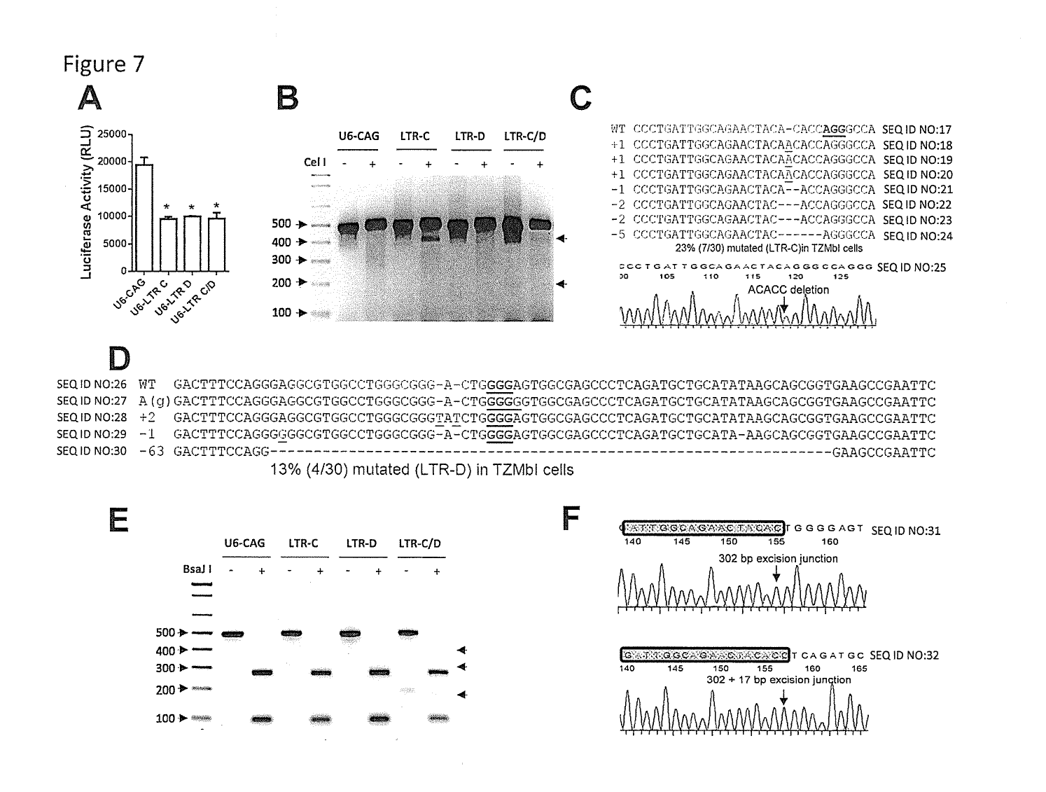

We assessed the ability of HIV-1-directed guide RNAs (gRNAs) to abrogate LTR transcriptional activity and eradicate proviral DNA from the genomes of latently-infected myeloid cells that serve as HIV-1 reservoirs in the brain, a particularly intractable target population. Our strategy was focused on targeting the HIV-1 LTR promoter U3 region. By bioinformatic screening and efficiency/off-target prediction, we identified four gRNA targets (protospacers; LTRs A-D) that avoid conserved transcription factor binding sites, minimizing the likelihood of altering host gene expression (FIGS. 5 and 13). We inserted DNA fragments complementary to gRNAs A-D into a humanized Cas9 expression vector (A/B in pX260; C/D in pX330) and tested their individual and combined abilities to alter the integrated HIV-1 genome activity. We first utilized the microglial cell line CHME5, which harbors integrated copies of a single round HIV-1 vector that includes the 5′ and 3′ LTRs, and a gene encoding an enhanced green fluorescent protein (EGFP) reporter replacing Gag (pNL4-3-ΔGag-d2EGFP). Treating CHME5 cells with trichostatin A (TSA), a histone deacetylase inhibitor, reactivates transcription from the majority of the integrated proviruses and leads to expression of EG FP and the remaining HIV-1 proteome. Expressing of gRNAs plus Cas9 markedly decreased the fraction of TSA-induced EGFP-positive CHME5 cells (FIGS. 1A and 6). We detected insertion/deletion gene mutations (indels) for LTRs A-D (FIGS. 1B and 6B) using a Cel I nuclease-based heteroduplex-specific SURVEYOR assay. Similarly, expressing gRNAs targeting LTRs C and D in HeLa-derived TZM-bI cells, that contain stably incorporated HIV-1 LTR copies driving a firefly-luciferase reporter gene, suppressed viral promoter activity (FIG. 7A), and elicited indels within the LTR U3 region (FIG. 7B-D) demonstrated by SURVEYOR and Sanger sequencing. Moreover, the combined expression of LTR C/D-targeting gRNAs in these cells caused excision of the predicted 302-bp viral DNA sequence, and emergence of the residual 194-bp fragment (FIG. 7E-F).

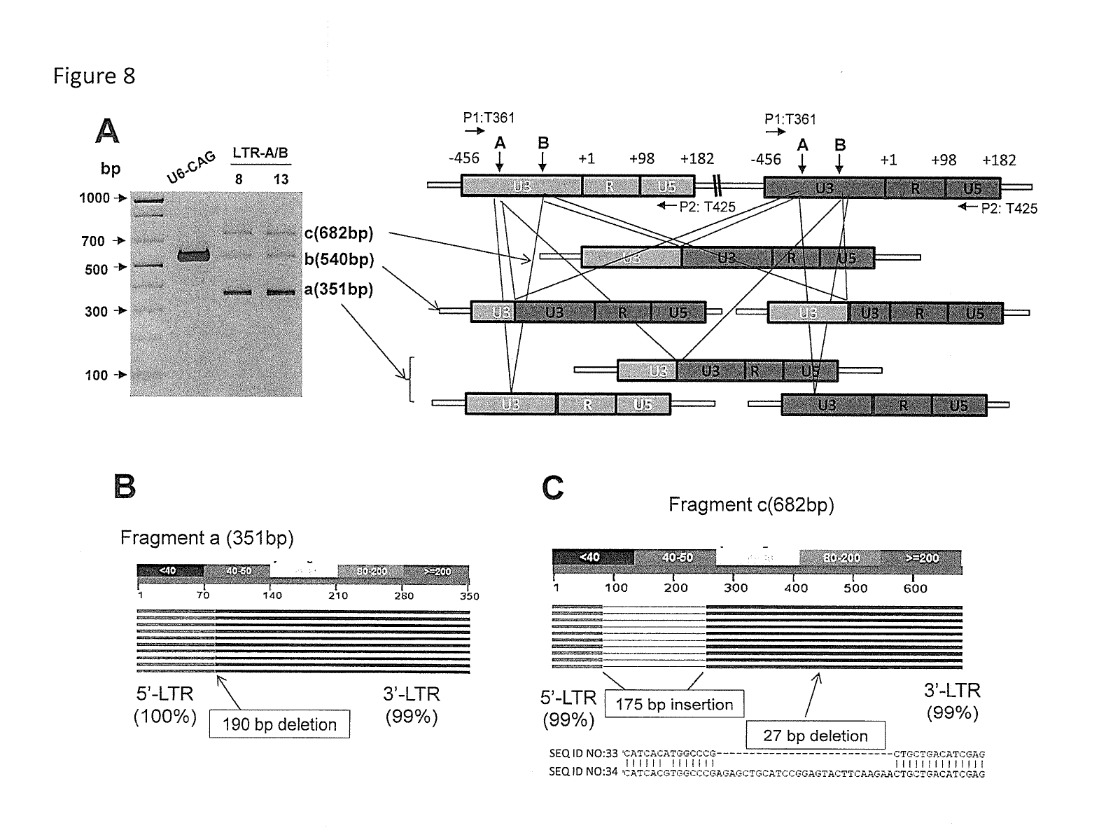

Multiplex expression of LTR-A/B gRNAs in mixed clonal CHME5 cells caused deletion of a 190-bp fragment between A and B target sites and led to indels to various extents (FIG. 1C-D). Among >20 puromycin-selected stable subclones, we found cell populations with complete blockade of TSA-induced HIV-1 proviral reactivation determined by flow cytometry for EGFP (FIG. 1E). PCR-based analysis for EGFP and HIV-1 Rev response element (RRE) in the proviral genome validated the eradication of HIV-1 genome (FIG. 1F, G). Furthermore, sequencing of the PCR products revealed the entire 5′-3′ LTR-spanning viral genome was deleted, yielding a 351-bp fragment via a 190-bp excision between cleavage sites A and B (FIGS. 1G and 8), and a 682-bp fragment with a 175-bp insertion and a 27-bp deletion at the LTR-A and -B sites respectively (FIG. 8C). The residual HIV-1 genome (FIG. 1F-H) may reflect the presence of trace Cas9/gRNA-negative cells. These results indicate that LTR-targeting Cas9/gRNAs A/B eradicates the HIV-1 genome and blocks its reactivation in latently infected microglial cells.

### Example 3: Cas9/LTR-gRNA Efficiently Eradicates Latent HIV-1 Virus from U1 Monocytic Cells

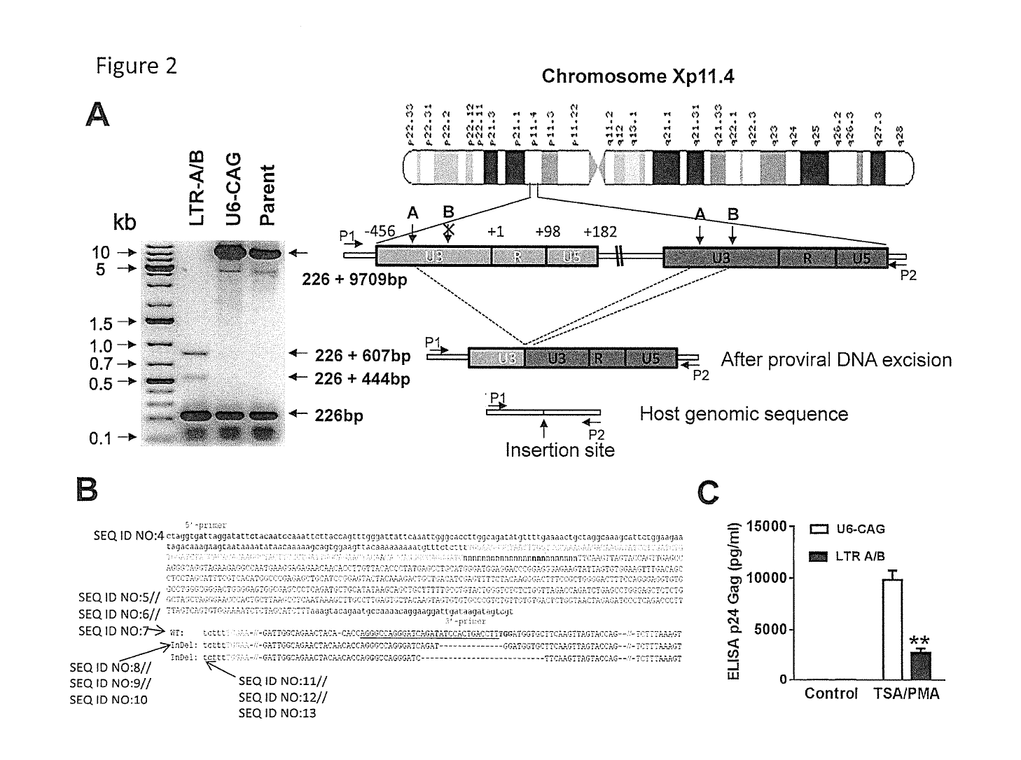

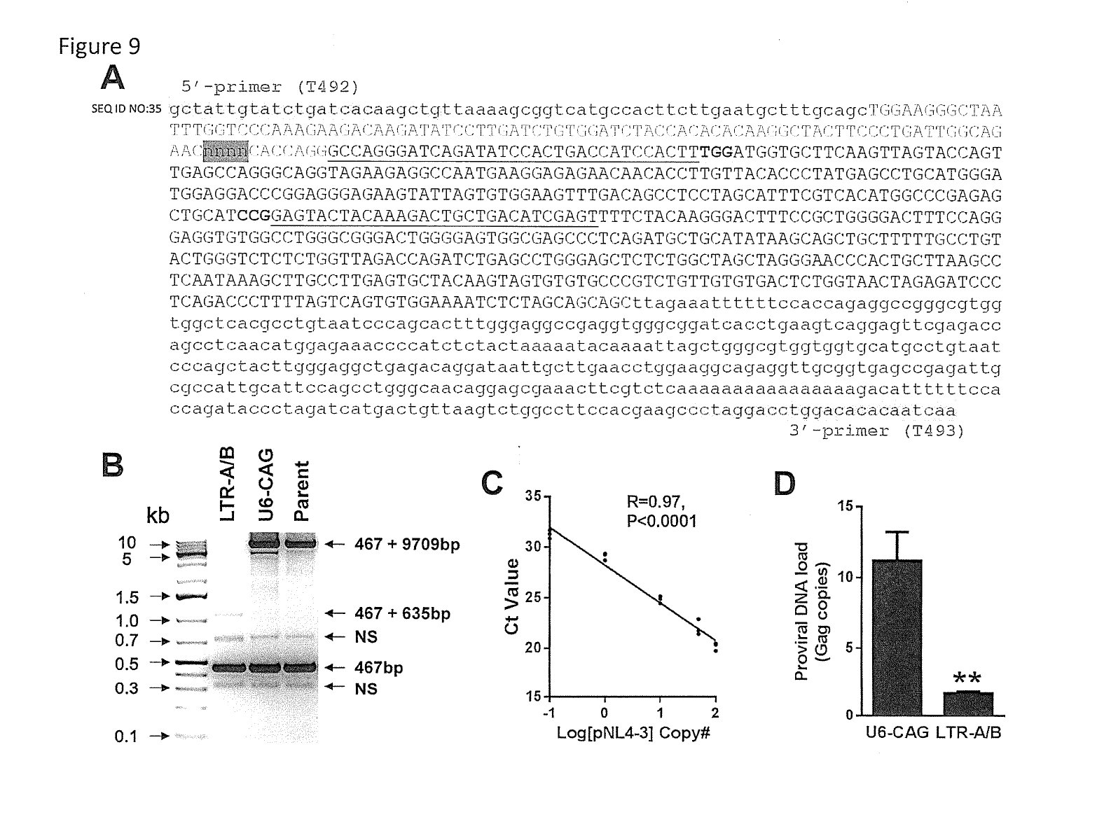

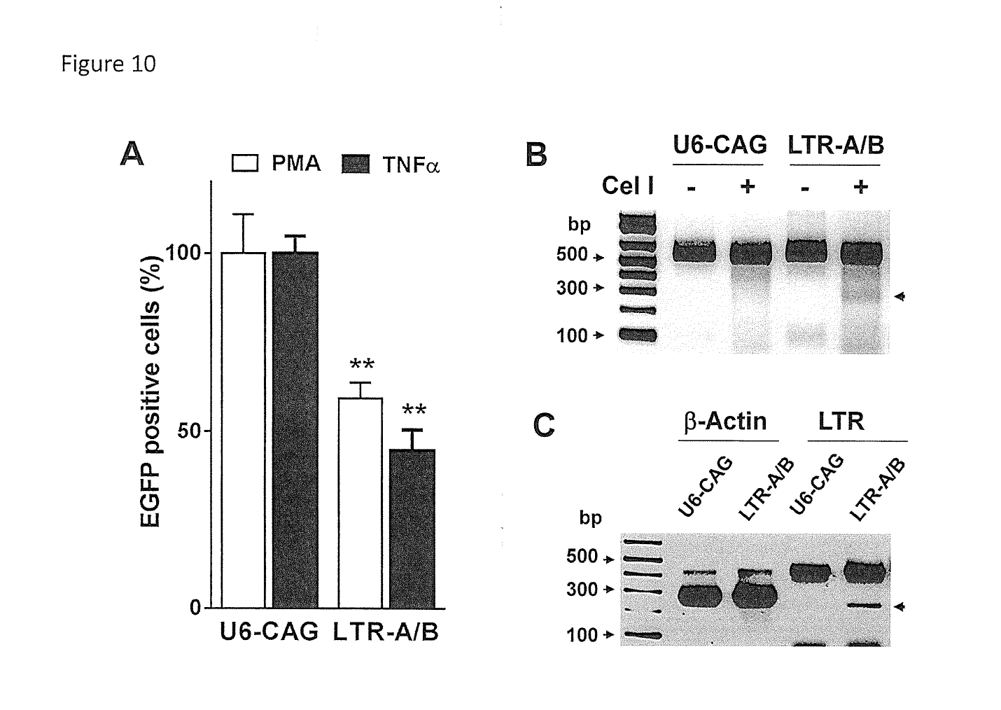

The promonocytic U-937 cell subclone U1, an HIV-1 latency model for infected perivascular macrophages and monocytes, is chronically HIV-1-infected and exhibits low level constitutive viral gene expression and replication. GenomeWalker mapping detected two integrated proviral DNA copies at chromosomes Xp11-4 (FIG. 2A) and 2p21 (FIG. 9A) in U1 cells. A 9935-bp DNA fragment representing the entire 9709-bp proviral HIV-1 DNA plus a flanking 226-bp X-chromosome-derived sequence (FIG. 2A), and a 10176-bp fragment containing 9709-bp HIV-1 genome plus its flanking 2-chromosome-derived 467-bp (FIG. 9A, B) were identified by the long-range PCR analysis of the parental control or empty-vector (U6-CAG) U1 cells. The 226-bp and 467-bp fragments represent the predicted segment from the other copy of chromosome X and 2 respectively, which lacked the integrated proviral DNA. In U1 cells expressing LTR-A/B gRNAs and Cas9, we found two additional DNA fragments of 833 and 670 bp in chromosome X and one additional 1102-bp fragment in chromosome 2. Thus, gRNAs A/B enabled Cas9 to excise the HIV-1 5′-3′ LTR-spanning viral genome segment in both chromosomes. The 833-bp fragment includes the expected 226-bp from the host genome and a 607-bp viral LTR sequence with a 27-bp deletion around the LTR-A site (FIG. 2A-B). The 670-bp fragment encompassed a 226-bp host sequence and residual 444-bp viral LTR sequence after 190-bp fragment excision (FIG. 1D), caused by gRNAs-A/B-guided cleavage at both LTRs (FIG. 2A). The additional fragments did not emerge via circular LTR integration, because it was absent in the parental U1 cells, and such circular LTR viral genome configuration occurs immediately after HIV-1 infection but is short lived and intolerant to repeated passaging. These cells exhibited substantially decreased HIV-1 viral load, shown by the functional p24 ELISA replication assay (FIG. 2C) and real-time PCR analysis (FIG. 9C, FIG. 9D). The detectable but low residual viral load and reactivation may result from cell population heterogeneity and/or incomplete genome editing. We also validated the ablation of HIV-1 genome by Cas9/LTR-A/B gRNAs in latently infected J-Lat T cells harboring integrated HIV-R7/E-/EGFP using flow cytometry analysis, SURVEYOR assay and PCR genotyping (FIG. 10), supporting the results of previous reports on HIV-1 proviral deletion in Jurkat T cells by Cas9/gRNA and ZFN. Taken together, our results suggest that the multiplex LTR-gRNAs/Cas9 system efficiently suppress HIV-1 replication and reactivation in latently HIV-1-infected “reservoir” (microglial, monocytic and T) cells typical of human latent HIV-1 infection, and in TZM-bI cells highly sensitive for detecting HIV-1 transcription and reactivation. Single or multiplex gRNAs targeting 5′- and 3′-LTRs effectively eradicated the entire HIV-1 genome.

### Example 4: Stable Expression of Cas9 Plus LTR-A/B Vaccinates TZM-bI Cells Against New HIV-1 Virus Infection

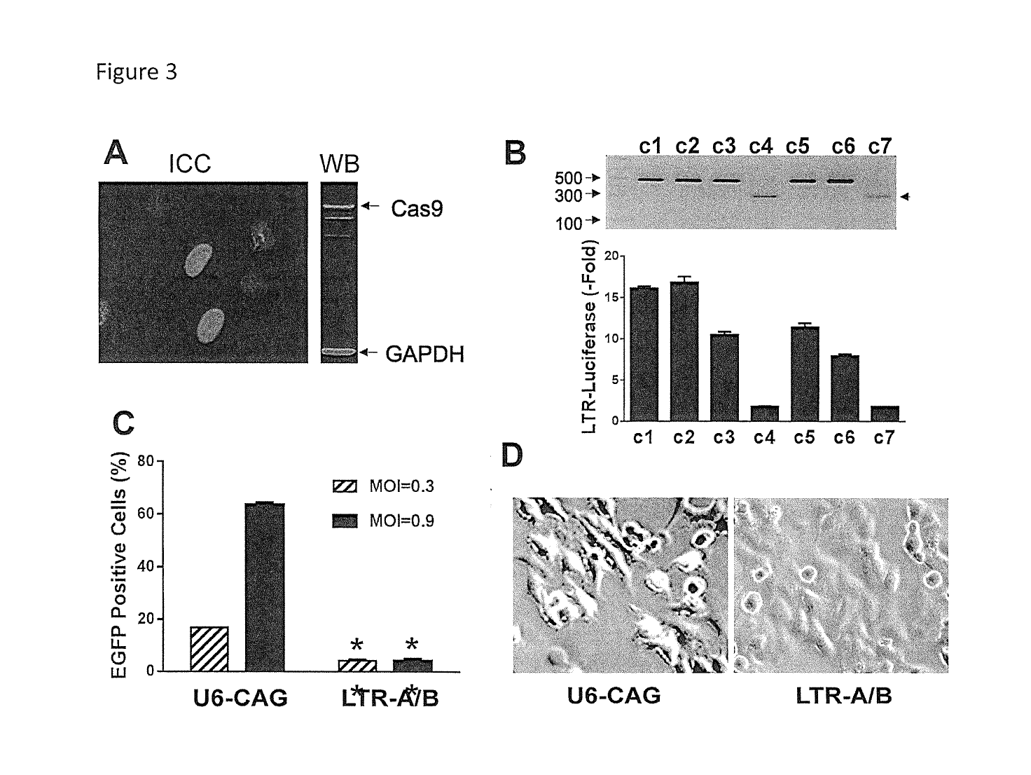

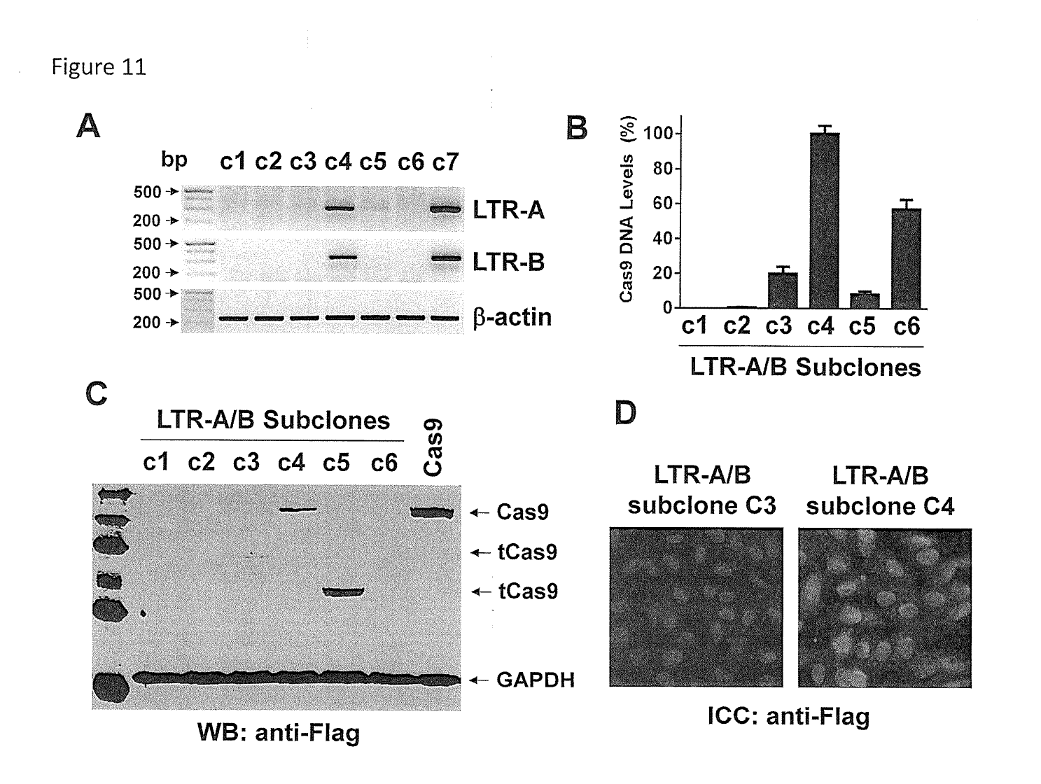

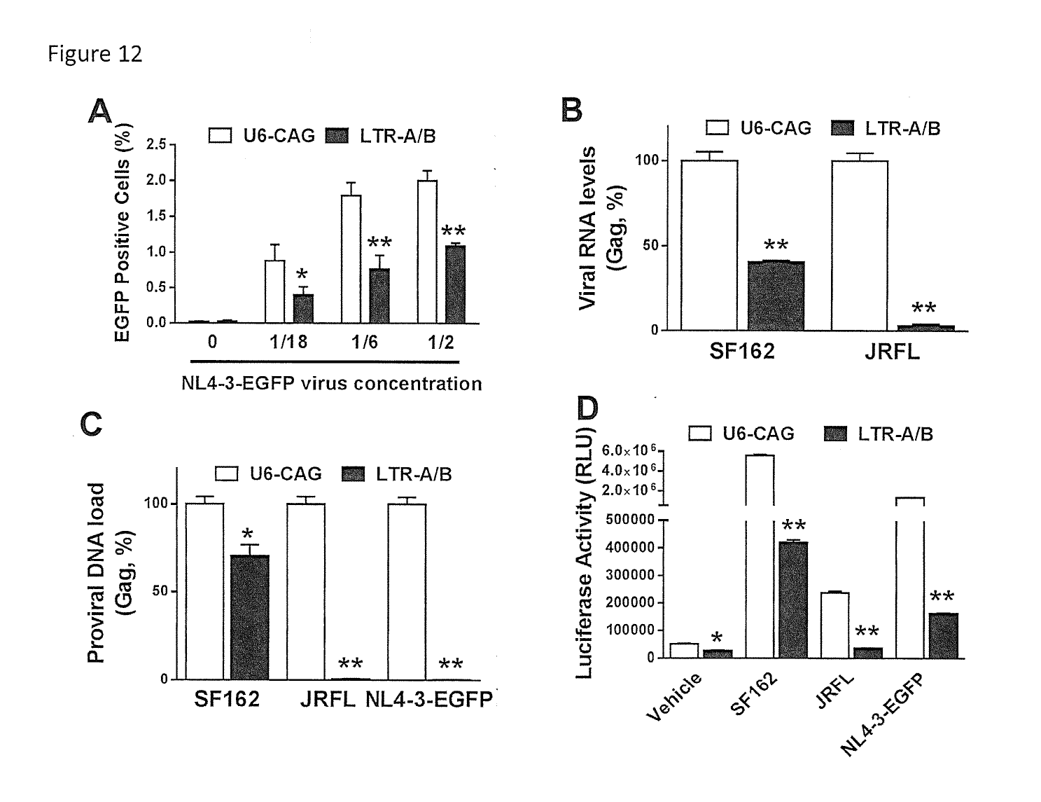

We next tested whether combined Cas9/LTR gRNAs can immunize cells against HIV-1 infection using stable Cas9/gRNAs-A and -B-expressing TZM-bI-based clones (FIG. 3A). Two of 7 puromycin-selected subclones exhibited efficient excision of the 190-bp LTR-A/B site-spanning DNA fragment (FIG. 3B). However, the remaining 5 subclones exhibited no excision (FIG. 3B) and no indel mutations as verified by Sanger sequencing. PCR genotyping using primers targeting Cas9 and U6-LTR showed that none of these ineffective subclones retained the integrated copies of Cas9/LTR-A/B gRNA expression cassettes. (FIG. 11A, FIG. 11B). As a result, no expression of full-length Cas9 was detected (FIG. 11C, FIG. 11D). The long-term expression of Cas9/LTR-A/B gRNAs did not adversely affect cell growth or viability, suggesting a low occurrence of off-target interference with the host genome or Cas9-induced toxicity in this model. We assessed de novo HIV-1 replication by infecting cells with the VSVG-pseudotyped pNL4-3-ΔE-EGFP reporter virus, with EGFP-positivity by flow cytometry indicating HIV-1 replication. Unlike the control U6-CAG cells, the cells stably expressing Cas9/gRNAs LTRs-A/B failed to support HIV-1 replication at 2 d post infection, indicating that they were immunized effectively against new HIV-1 infection (FIG. 3C and FIG. 3D). A similar immunity against HIV-1 was observed in Cas/LTR-A/B gRNA expressing cells infected with native T-tropic X4 strain pNL4-3-ΔE-EGFP reporter virus (FIG. 12A) or native M-tropic R5 strains such as SF162 and JRFL (FIG. 12B, FIG. 12C, and FIG. 12D).

### Example 5: Off-Target Effects of Cas9/LTR-A/B on Human Genome

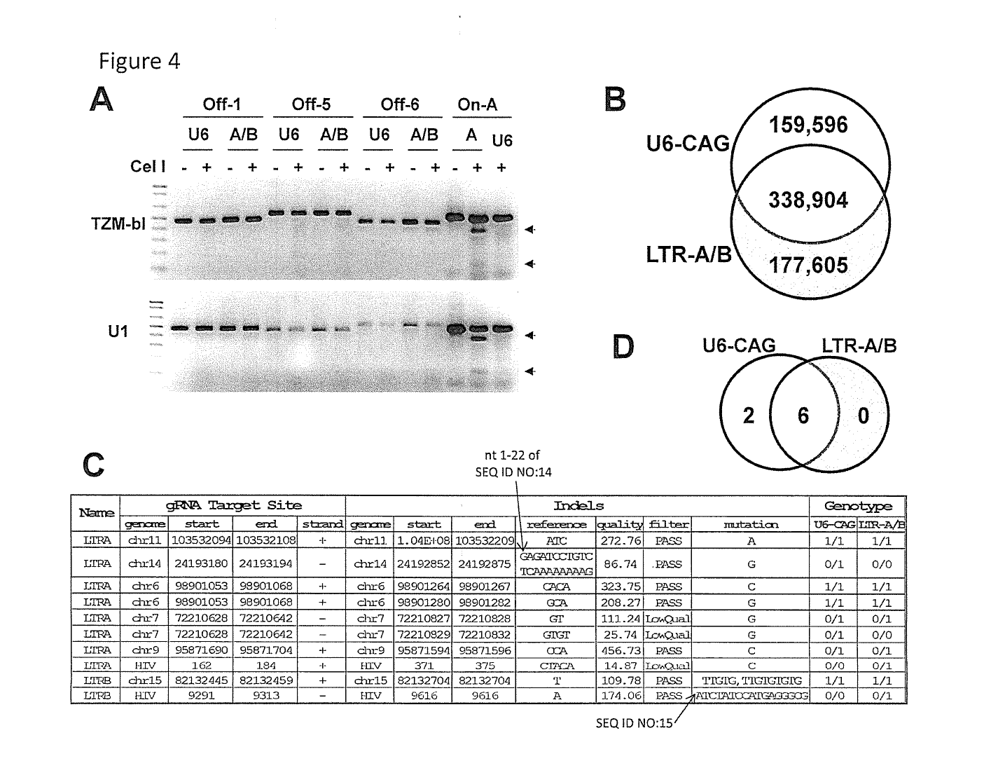

The appeal of Cas9/gRNA as an interventional approach rests on its highly specific on-target indel-producing cleavage, but multiplex gRNAs could potentially cause host genome mutagenesis and chromosomal disorders, cytotoxicity, genotoxicity, or oncogenesis. Fairly low viral-human genome homology reduces this risk, but the human genome contains numerous endogenous retroviral genomes that are potentially susceptible to HIV-1-directed gRNAs. Therefore, we assessed off-target effects of selected HIV-1 LTR gRNAs on the human genome. Because the 12-14-bp seed sequence nearest the protospacer-adjacent motif (PAM) region (NGG) is critical for cleavage specificity, we searched >14-bp seed+NGG, and found no off-target candidate sites by LTR gRNAs A-D (FIG. 13). It is not surprising that progressively shorter gRNA segments yielded increasing off-target cleavage sites 100% matched to corresponding on-target sequences (i.e., NGG+13 bp yielded 6, 0, 2 and 9 off-target sites, respectively, whereas NGG+12 bp yielded 16, 5, 16 and 29; FIG. 13). From human genomic DNA we obtained a 500-800-bp sequence covering one of predicted off-target sites using high-fidelity PCR, and analyzed the potential mutations by SURVEYOR and Sanger sequencing. We found no mutations (see representative off-target sites #1, 5 and 6 in TZM-bI and U1 cells; FIG. 4A).

To assess risk of off-target effects comprehensively, we performed whole genome sequencing (WGS) using the stable Cas9/gRNA A/B-expressing and control U6-CAG TZM-bI cells (FIG. 4B, FIG. 4C, and FIG. 4D). We identified 676,105 indels, using a genome analysis toolkit (GATK, v.2.8.1) with human (hg19) and HIV-1 genomes as reference sequences. Among the indels, 24% occurred in the U6-CAG control, 26% in LTR-A/B subclone, and 50% in both (FIG. 4B). Such substantial inter-sample indel-calling discrepancy suggests the probable off-target effects, but most likely results from its limited confidence, limited WGS coverage (15-30×), and cellular heterogeneity. GATK reported only confidently-identified indels: some found in the U6-CAG control but not in the LTR-A/B subclone, and others in the LTR-A/B but not in the U6-CAG. We expected abundant missing indel calls for both samples due to the limited WGS coverage. Such limited indel-calling confidence also implies the possibility of false negatives: missed indels occurring in LTR-A/B but not U6-CAG controls. Cellular heterogeneity may reflect variability of Cas9/gRNA editing efficiency and effects of passaging. Therefore, we tested whether each indel was LTR-A/B gRNA-induced, by analyzing ±300 bp flanking each indel against LTRs-A/− B-targeted sites of the HIV-1 genome and predicted/potential gRNA off-target sites of the host genome (FIG. 15). For sequences 100% matched to one containing the seed (12-bp) plus NRG, we identified only 8 overlapped regions of 92 potential off-target sites against 676,105 indels: 6 indels occurring in both samples, and 2 only in the U6-CAG control (FIG. 4C, FIG. 4D). We also identified 2 indels on HIV-1 LTR that occurred only in the LTR-A/B subclone but, as expected, not in the U6-CAG control (FIG. 4C). The results suggest that LTR-A/B gRNAs induce the indicated on-target indels, but no off-target indels, consistent with prior findings using deep sequencing of PCR products covering predicted/potential off-target site.

Our combined approaches minimized off-target effects while achieving high efficiency and complete ablation of the genomically integrated HIV-1 provirus. In addition to an extremely low homology between the foreign viral genome and host cellular genome including endogenous retroviral DNA, the key design attributes in our study included: bioinformatic screening using the strictest 12-bp+NGG target-selection criteria to exclude off-target human transcriptome or (even rarely) untranslated-genomic sites; avoiding transcription factor binding sites within the HIV-1 LTR promoter (potentially conserved in the host genome); selection of LTR-A- and -B-directed, 30-bp gRNAs and also pre-crRNA system reflecting the original bacterial immune mechanism to enhance specificity/efficiency vs. 20-bp gRNA-, chimeric crRNA-tracRNA-based system; and WGS, Sanger sequencing and SURVEYOR assay, to identify and exclude potential off-target effects. Indeed, the use of newly developed Cas9 double-nicking and RNA-guided FokI nuclease may further assist identification of new targets within the various conserved regions of HIV-1 with reduced off-target effects.

Our results show that the HIV-1 Cas9/gRNA system has the ability to target more than one copy of the LTR, which are positioned on different chromosomes, suggesting that this genome editing system can alter the DNA sequence of HIV-1 in latently infected patient's cells harboring multiple proviral DNAs. To further ensure high editing efficacy and consistency of our technology, one may consider the most stable region of HIV-1 genome as a target to eradicate HIV-1 in patient samples, which may not harbor only one strain of HIV-1. Alternatively, one may develop personalized treatment modalities based on the data from deep sequencing of the patient-derived viral genome prior to engineering therapeutic Cas9/gRNA molecules.

Our results also demonstrate that Cas9/gRNA genome editing can be used to immunize cells against HIV-1 infection. The preventative vaccination is independent of HIV-1 strain's diversity because the system targets genomic sequences regardless of how the viruses enter the infected cells. The preexistence of the Cas9/gRNA system in cells led to a rapid elimination of the new HIV-1 before it integrates into the host genome. One may explore various systems for delivery of Cas9/LTR-gRNA for immunizing high-risk subjects, e.g., gene therapies (viral vector and nanoparticle) and transplantation of autologous Cas9/gRNA-modified bone marrow stem/progenitor cells or inducible pluripotent stem cells for eradicating HIV-1 infection.

Here, we demonstrated the high specificity of Cas9/gRNAs in editing HIV-1 target genome. Results from subclone data revealed the strict dependence of genome editing on the presence of both Cas9 and gRNA. Moreover, only one nucleotide mismatch in the designed gRNA target will disable the editing potency. In addition, all of our 4 designed LTR gRNAs worked well with different cell lines, indicating that the editing is more efficient in the HIV-1 genome than the host cellular genome, wherein not all designed gRNAs are functional, which may be due to different epigenetic regulation, variable genome accessibility, or other reasons. Given the ease and rapidity of Cas9/gRNA development, even if HIV-1 mutations confer resistance to one Cas9/gRNA-based therapy, as described above, HIV-1 variants can be genotyped to enable another personalized therapy for individual patients.

A number of embodiments of the invention have been described. Nevertheless, it will be understood that various modifications may be made without departing from the spirit and scope of the invention. Accordingly, other embodiments are within the scope of the following claims.

### REFERENCES

Alvarez-Erviti L, Seow Y, Yin H, Betts C, Lakhal S, M J A Wood. Delivery of siRNA to the mouse brain by systemic injection of targeted exosomes. Nature Biotechnol. 2011, 29: 341-345.
Kleinstiver B P, Pattanayak, Prew M S, Tsai S Q Nguyen N T, Zheng Z, Joung J K. High-fidelity CRISPR-Cas9 nucleases with no detectable genome-wide off-target effects, Nature (2016). DOI: 10.1038/nature16526.
Kooijmans S A A, Vader P, Dommelen S M, van Solinge W W, Raymond M Schiffelers R M. Exosome mimetics: a novel class of drug delivery systems. Int. J. Nanomed. 2012, 7: 1525-1541.
Marcus M E, and Leonard J N. FedExosomes: Engineering therapeutic biological nanoparticles that truly deliver. Pharmaceuticals 2103, 6: 659-680.
Shtam T A, Kovalev R A, Varfolomeeva E Y, Makarov E M, Kil Y V, Filatov M V. Exosomes are natural carriers of exogenous siRNA to human cells in vitro. Cell Communication and Signaling 2013, 11:88 (http://www.biosignaling.com/content/11/1/88).
Slaymaker I M, Gao L, Zetsche B, Scott D A, Yan W X, Feng Zhang F. Rationally engineered Cas9 nucleases with improved specificity. Published online 1 Dec. 2015 [DOI:10.1126/science.aad5227].
Sun W, Jiang T, Yue Lu Y, Reiff M, Mo R, Zhen Gu Z. Cocoon-like self-degradable DNA nanoclew for anticancer drug delivery. J. Am. Chem. Soc. 2014, 136:14722-14725.
Sun W, Ji W, Hall J M, Hu Q, Wang C, Beisel C L, Gu Z. Self-assembled DNA nanoclews for the efficient delivery of CRISPR-Cas9 for genome editing. Angew. Chem. Int. Ed. 2015: 12029-12033.

What is claimed is:

1. A method of inactivating a proviral DNA integrated into the genome of a host cell latently infected with a retrovirus, including the steps of:
treating the host cell with a composition comprising a Clustered Regularly Interspaced Short Palindromic Repeat (CRISPR)-associated endonuclease, and two or more different guide RNAs (gRNAs), wherein each of the at least two gRNAs is complementary to a different target nucleic acid sequence in a long terminal repeat (LTR) of the proviral DNA; and
inactivating the proviral DNA.

2. The method according to claim 1, wherein said step of treating the host cell includes the steps of:
exposing the host cell to a composition including an isolated nucleic acid encoding the CRISPR-associated endonuclease; an isolated nucleic acid sequence encoding a first gRNA having a first spacer sequence that is complementary to a first target protospacer sequence in a proviral DNA; and an isolated nucleic acid encoding a second gRNA having a second spacer sequence that is complementary to a second target protospacer sequence in the proviral DNA;
expressing in the host cell the CRISPR-associated endonuclease, the first gRNA, and the second gRNA;
assembling, in the host cell, a first gene editing complex including the CRISPR-associated endonuclease and the first gRNA; and a second gene editing complex including the CRISPR-associated endonuclease and the second gRNA;
directing the first gene editing complex to the first target protospacer sequence by complementary base pairing between the first spacer sequence and the first target protospacer sequence;
directing the second gene editing complex to the second target protospacer sequence by complementary base pairing between the second spacer sequence and the second target protospacer sequence;
cleaving the proviral DNA at the first target protospacer sequence with the CRISPR-associated endonuclease;
cleaving the proviral DNA at the second target protospacer sequence with the CRISPR-associated endonuclease; and
inducing at least one mutation in the proviral DNA.

3. The method according to claim 2, wherein at least one of the first target protospacer sequence and the second target protospacer sequence is situated within the U3 region of the LTR.

4. The method according to claim 3, wherein the retrovirus is selected from the group consisting of HIV-1, HIV-2, and SIV.

5. The method according to claim 4, wherein the retrovirus is HIV-1, and the first spacer sequence and the second spacer sequence each include a sequence complementary to a target protospacer sequence selected from the group consisting of SEQ ID NO: 96, SEQ ID NO: 121, SEQ ID NO: 87, and SEQ ID NO: 110.

6. The method according to claim 4, wherein the retrovirus is HIV-1, and the first spacer sequence and the second spacer sequence include, respectively, a sequence complementary to the target protospacer sequences SEQ ID NO: 96 and SEQ ID NO: 121.

7. The method according to claim 4, wherein the retrovirus is HIV-1, and the first spacer sequence and the second spacer sequence each include, respectively, a sequence complementary to the target protospacer sequences SEQ ID NO: 87 and SEQ ID NO: 110.

8. The method according to claim 1, wherein the CRISPR-associated endonuclease is selected from a wild-type Cas9; a human-optimized Cas9; a nickase mutant Cas9, SpCas9(K855A); SpCas9(K810A/K1003A/R1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E, M694A.

9. The method according to claim 2, wherein the isolated nucleic acids encoding a CRISPR-associated endonuclease, the first gRNA, and the second gRNA, are encoded in at least one expression vector.

10. The method according to claim 9, wherein the at least one expression vector is selected from the group consisting of a plasmid vector, a lentiviral vector, an adenoviral vector, and an adeno-associated virus vector.

11. The method according to claim 1, wherein at least one of the gRNAs comprises a CRISPR RNA (crRNA) and a trans-activated small RNA (tracrRNA), which are expressed as separate nucleic acids.

12. The method according to claim 1, wherein at least one of the gRNAs is engineered as an artificial fusion small guide RNA (sgRNA) comprised of a crRNA and a tracrRNA.

13. The method according to claim 2, wherein said step of inducing at least one mutation in the proviral RNA is further defined as inducing at least one insertion, at least one deletion, at least one point mutation, or a combination thereof.

14. The method according to claim 1, wherein said step of inducing at least one mutation in the proviral RNA is further defined as inducing the excision of a proviral DNA sequence that extends between the first target protospacer sequence and the second target protospacer sequence.

15. The method of claim 1, wherein said step of expressing in the host cell the CRISPR-associated endonuclease, the first gRNA, and the second gRNA, is further defined as stably expressing in the host cell the CRISPR-associated endonuclease, the first gRNA, and the second gRNA, and the method additionally includes the step of immunizing the host cell against new retroviral infection.

16. The method according to claim 1, wherein the host cell latently infected with a retrovirus is a CD4+ T cell, a macrophage, a monocyte, a gut associated lymphoid cell, a microglial cell, or an astrocyte.

17. A composition for use in inactivating a proviral DNA integrated into the genome of a host cell latently infected with a retrovirus comprising:
an isolated nucleic acid encoding a Clustered Regularly Interspaced Short Palindromic Repeat (CRISPR)-associated endonuclease;
an isolated nucleic acid sequence encoding a first guide RNA (gRNA) having a first spacer sequence that is complementary to a first target protospacer sequence in a proviral DNA; and
an isolated nucleic acid sequence encoding a second gRNA having a second spacer sequence that is complementary to a second target protospacer sequence in the proviral DNA, wherein said first target protospacer sequence and said second target protospacer sequence are situated in a long terminal repeat (LTR) of the proviral DNA.

18. The composition according to claim 17, wherein said first spacer sequence and said second spacer sequence each include a sequence complementary to a target protospacer sequence selected from the group consisting of SEQ ID NO: 96, SEQ ID NO: 121, SEQ ID NO: 87, and SEQ ID NO: 110.

19. The composition according to claim 17, wherein said first spacer sequence and said second spacer sequence include, respectively, a sequence complementary to target protospacer sequences SEQ ID NO: 96 and SEQ ID NO: 121.

20. The composition according to claim 17, wherein said first spacer sequence and said second spacer sequence each include, respectively, a sequence complementary to target protospacer sequences SEQ ID NO: 87 and SEQ ID NO: 110.

21. The composition according to claim 17, wherein said CRISPR-associated endonuclease is selected from a wild-type Cas9; a human-optimized Cas9; a nickase mutant Cas9, SpCas9(K855A); SpCas9(K810A/K1003A/R1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; and SpCas9 R661A, Q695A, Q926A, D1135E, M694A.


SEQUENCE LISTING (389 sequences)

SEQ ID NO: 1 - DNA, 30 bp, Human immunodeficiency virus 1
gccagggatcagatatccactgacctttgg

SEQ ID NO: 2 - DNA, 34 bp, Human immunodeficiency virus 1
tccggagtacttcaagaactgctgacatcgagct

SEQ ID NO: 3 - DNA, 19 bp, Artificial Sequence
  Description of Artificial Sequence Synthetic oligonucleotide
ccactgactacttcaagaa

SEQ ID NO: 4 - DNA, 859 bp, Artificial Sequence
  Description of Artificial Sequence Synthetic polynucleotide
  modified_base (289)..(313): a, c, t, g, unknown or other
  misc_feature (289)..(313): n is a, c, g, or t
ctaggtgattaggatattctacaatccaaattcttaccagtttgggattattcaaattgggcaccttggcagatatgttttgaaaactgctaggcaaagcattctggaagaatagacaaagaagtaataaaatataacaaaaagcagtggaagttacaaaaaaaaatgtttctcttttggaagggctaatttggtcccaaagaagacaagatatccttgatctgtggatctaccacacacaaggctacttccctgattggcagaactacaacaccagggccagggatcnnnnnnnnnnnnnnnnnnnnnnnnnttcaagttagtaccagttgagccagggcaggtagaagaggccaatgaaggagagaacaacaccttgttacaccctatgagcctgcatgggatggaggacccggagggagaagtattagtgtggaagtttgacagcctcctagcatttcgtcacatggcccgagagctgcatccggagtactacaaagactgctgacatcgagttttctacaagggactttccgctggggactttccagggaggtgtggcctgggcgggactggggagtggcgagccctcagatgctgcatataagcagctgctttttgcctgtactgggtctctctggttagaccagatctgagcctgggagctctctggctagctagggaacccactgcttaagcctcaataaagcttgccttgagtgctacaagtagtgtgtgcccgtctgttgtgtgactctggtaactagagatccctcagacccttttagtcagtgtggaaaatctctagcatctttaaagtacagaatgccaaaacaggaaggattgataagatagtcgt

SEQ ID NO: 5 - DNA, 10 bp, Human immunodeficiency virus 1
tcttttggaa

SEQ ID NO: 6 - DNA, 76 bp, Human immunodeficiency virus 1
gattggcagaactacacaccagggccagggatcagatatccactgacctttggatggtgcttcaagttagtaccag

SEQ ID NO: 7 - DNA, 10 bp, Human immunodeficiency virus 1
tctttaaagt

SEQ ID NO: 8 - DNA, 10 bp, Artificial Sequence
  Description of Artificial Sequence Synthetic oligonucleotide
tcttttggaa

SEQ ID NO: 9 - DNA, 63 bp, Artificial Sequence
  Description of Artificial Sequence Synthetic oligonucleotide
gattggcagaactacaacaccagggccagggatcagatggatggtgcttcaagttagtaccag

SEQ ID NO: 10 - DNA, 10 bp, Artificial Sequence
  Description of Artificial Sequence Synthetic oligonucleotide
tctttaaagt

SEQ ID NO: 11 - DNA, 10 bp, Artificial Sequence
  Description of Artificial Sequence Synthetic oligonucleotide
tcttttggaa

SEQ ID NO: 12 - DNA, 50 bp, Artificial Sequence
  Description of Artificial Sequence Synthetic oligonucleotide
gattggcagaactacaacaccagggccagggatcttcaagttagtaccag

SEQ ID NO: 13 - DNA, 10 bp, Artificial Sequence
  Description of Artificial Sequence Synthetic oligonucleotide
tctttaaagt

SEQ ID NO: 14 - DNA, 24 bp, Artificial Sequence
  Description of Artificial Sequence Synthetic oligonucleotide
gagatcctgtctcaaaaaaaagtt

SEQ ID NO: 15 - DNA, 17 bp, Artificial Sequence
  Description of Artificial Sequence Synthetic oligonucleotide
atctatccatgagggcg

SEQ ID NO: 16 - DNA, 402 bp, Human immunodeficiency virus 1
gatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatgctgcatataagcagctgctttt

SEQ ID NO: 17 - DNA, 31 bp, Human immunodeficiency virus 1
ccctgattggcagaactacacaccagggcca

SEQ ID NO: 18 - DNA, 32 bp, Artificial Sequence
  Description of Artificial Sequence Synthetic oligonucleotide
ccctgattggcagaactacaacaccagggcca

SEQ ID NO: 19 - DNA, 32 bp, Artificial Sequence; identical to SEQ ID NO: 18
  Description of Artificial Sequence Synthetic oligonucleotide

SEQ ID NO: 20 - DNA, 32 bp, Artificial Sequence; identical to SEQ ID NO: 18
  Description of Artificial Sequence Synthetic oligonucleotide

SEQ ID NO: 21 - DNA, 30 bp, Artificial Sequence
  Description of Artificial Sequence Synthetic oligonucleotide
ccctgattggcagaactacaaccagggcca

SEQ ID NO: 22 - DNA, 29 bp, Artificial Sequence
  Description of Artificial Sequence Synthetic oligonucleotide
ccctgattggcagaactacaccagggcca

SEQ ID NO: 23 - DNA, 29 bp, Artificial Sequence
  Description of Artificial Sequence Synthetic oligonucleotide
ccctgattggcagaactacaccagggcca

SEQ ID NO: 24 - DNA, 26 bp, Artificial Sequence
  Description of Artificial Sequence Synthetic oligonucleotide
ccctgattggcagaactacagggcca

SEQ ID NO: 25 - DNA, 29 bp, Artificial Sequence
  Description of Artificial Sequence Synthetic oligonucleotide
ccctgattggcagaactacagggccaggg

SEQ ID NO: 26 - DNA, 86 bp, Human immunodeficiency virus 1
gactttccagggaggcgtggcctgggcgggactggggagtggcgagccctcagatgctgcatataagcagcggtgaagccgaattc

SEQ ID NO: 27 - DNA, 86 bp, Artificial Sequence
  Description of Artificial Sequence Synthetic oligonucleotide
gactttccagggaggcgtggcctgggcgggactggggggtggcgagccctcagatgctgcatataagcagcggtgaagccgaattc

SEQ ID NO: 28 - DNA, 88 bp, Artificial Sequence
  Description of Artificial Sequence Synthetic oligonucleotide
gactttccagggaggcgtggcctgggcgggtatctggggagtggcgagccctcagatgctgcatataagcagcggtgaagccgaattc

SEQ ID NO: 29 - DNA, 85 bp, Artificial Sequence
  Description of Artificial Sequence Synthetic oligonucleotide
gactttccaggggggcgtggcctgggcgggactggggagtggcgagccctcagatgctgcataaagcagcggtgaagccgaattc

SEQ ID NO: 30 - DNA, 23 bp, Artificial Sequence
  Description of Artificial Sequence Synthetic oligonucleotide
gactttccagggaagccgaattc

SEQ ID NO: 31 - DNA, 25 bp, Artificial Sequence
  Description of Artificial Sequence Synthetic oligonucleotide
gattggcagaactacactggggagt

SEQ ID NO: 32 - DNA, 26 bp, Artificial Sequence
  Description of Artificial Sequence Synthetic oligonucleotide
gattggcagaactacacctcagatgc

SEQ ID NO: 33 - DNA, 28 bp, Artificial Sequence
  Description of Artificial Sequence Synthetic oligonucleotide
catcacatggcccgctgctgacatcgag

SEQ ID NO: 34 - DNA, 55 bp, Human immunodeficiency virus 1
catcacgtggcccgagagctgcatccggagtacttcaagaactgctgacatcgag

SEQ ID NO: 35 - DNA, 1106 bp, Artificial Sequence
  Description of Artificial Sequence Synthetic polynucleotide
  modified_base (152)..(155): a, c, t, g, unknown or other
  misc_feature (152)..(155): n is a, c, g, or t
gctattgtatctgatcacaagctgttaaaagcggtcatgccacttcttgaatgctttgcagctggaagggctaatttggtcccaaagaagacaagatatccttgatctgtggatctaccacacacaaggctacttccctgattggcagaacnnnncaccagggccagggatcagatatccactgaccatccactttggatggtgcttcaagttagtaccagttgagccagggcaggtagaagaggccaatgaaggagagaacaacaccttgttacaccctatgagcctgcatgggatggaggacccggagggagaagtattagtgtggaagtttgacagcctcctagcatttcgtcacatggcccgagagctgcatccggagtactacaaagactgctgacatcgagttttctacaagggactttccgctggggactttccagggaggtgtggcctgggcgggactggggagtggcgagccctcagatgctgcatataagcagctgctttttgcctgtactgggtctctctggttagaccagatctgagcctgggagctctctggctagctagggaacccactgcttaagcctcaataaagcttgccttgagtgctacaagtagtgtgtgcccgtctgttgtgtgactctggtaactagagatccctcagacccttttagtcagtgtggaaaatctctagcagcagcttagaaattttttccaccagaggccgggcgtggtggctcacgcctgtaatcccagcactttgggaggccgaggtgggcggatcacctgaagtcaggagttcgagaccagcctcaacatggagaaaccccatctctactaaaaatacaaaattagctgggcgtggtggtgcatgcctgtaatcccagctacttgggaggctgagacaggataattgcttgaacctggaaggcagaggttgcggtgagccgagattgcgccattgcattccagcctgggcaacaggagcgaaacttcgtctcaaaaaaaaaaaaaaaagacattttttccaccagataccctagatcatgactgttaagtctggccttccacgaagccctaggacctggacacacaatcaa

SEQ ID NO: 36 - DNA, 36 bp, Artificial Sequence
  Description of Artificial Sequence Synthetic oligonucleotide
aaacagggccagggatcagatatccactgaccttgt

SEQ ID NO: 37 - DNA, 35 bp, Artificial Sequence
  Description of Artificial Sequence Synthetic oligonucleotide
taaacaaggtcagtggatatctgatccctggccct

SEQ ID NO: 38 - DNA, 36 bp, Artificial Sequence
  Description of Artificial Sequence Synthetic oligonucleotide
aaacagctcgatgtcagcagttcttgaagtactcgt

SEQ ID NO: 39 - DNA, 35 bp, Artificial Sequence
  Description of Artificial Sequence Synthetic oligonucleotide
taaacgagtacttcaagaactgctgacatcgagct

SEQ ID NO: 40 - DNA, 24 bp, Artificial Sequence
  Description of Artificial Sequence Synthetic oligonucleotide
caccgattggcagaactacacacc

SEQ ID NO: 41 - DNA, 24 bp, Artificial Sequence
  Description of Artificial Sequence Synthetic oligonucleotide
aaacggtgtgtagttctgccaatc

SEQ ID NO: 42 - DNA, 24 bp, Artificial Sequence
  Description of Artificial Sequence Synthetic oligonucleotide
caccgcgtggcctgggcgggactg

SEQ ID NO: 43 - DNA, 24 bp, Artificial Sequence
  Description of Artificial Sequence Synthetic oligonucleotide
aaaccagtcccgcccaggccacgc

SEQ ID NO: 44 - DNA, 24 bp, Artificial Sequence
  Description of Artificial Sequence Synthetic primer
tggaagggctaattcactcccaac

SEQ ID NO: 45 - DNA, 24 bp, Artificial Sequence
  Description of Artificial Sequence Synthetic primer
ccgagagctcccaggctcagatct

SEQ ID NO: 46 - DNA, 27 bp, Artificial Sequence
  Description of Artificial Sequence Synthetic primer
caccgatctgtggatctaccacacaca

SEQ ID NO: 47 - DNA, 24 bp, Artificial Sequence
  Description of Artificial Sequence Synthetic primer
aaacgagtcacacaacagacgggc

SEQ ID NO: 48 - DNA, 37 bp, Artificial Sequence
  Description of Artificial Sequence Synthetic primer
cgcctcgaggatccgagggcctatttcccatgattcc

SEQ ID NO: 49 - DNA, 35 bp, Artificial Sequence
  Description of Artificial Sequence Synthetic primer
tgtgaattcaggcgggccatttaccgtaagttatg

SEQ ID NO: 50 - DNA, 25 bp, Artificial Sequence
  Description of Artificial Sequence Synthetic primer
acgactatcttatcaatccttcctg

SEQ ID NO: 51 - DNA, 26 bp, Artificial Sequence
  Description of Artificial Sequence Synthetic primer
ctaggtgattaggatattctacaatc

SEQ ID NO: 52 - DNA, 24 bp, Artificial Sequence
  Description of Artificial Sequence Synthetic primer
gctattgtatctgatcacaagctg

SEQ ID NO: 53 - DNA, 24 bp, Artificial Sequence
  Description of Artificial Sequence Synthetic primer
ttgattgtgtgtccaggtcctagg

SEQ ID NO: 54 - DNA, 23 bp, Artificial Sequence
  Description of Artificial Sequence Synthetic primer
gcaagggcgaggagctgttcacc

SEQ ID NO: 55 - DNA, 24 bp, Artificial Sequence
  Description of Artificial Sequence Synthetic primer
ttgtagttgccgtcgtccttgaag

SEQ ID NO: 56 - DNA, 23 bp, Artificial Sequence
  Description of Artificial Sequence Synthetic primer
aatggtacatcaggccatatcac

SEQ ID NO: 57 - DNA, 23 bp, Artificial Sequence
  Description of Artificial Sequence Synthetic primer
cccactgtgtttagcatggtatt

SEQ ID NO: 58 - DNA, 23 bp, Artificial Sequence
  Description of Artificial Sequence Synthetic primer
cacagcatcaagaagaacctgat

SEQ ID NO: 59 - DNA, 24 bp, Artificial Sequence
  Description of Artificial Sequence Synthetic primer
tcttccgtctggtgtatcttcttc

SEQ ID NO: 60 - DNA, 28 bp, Artificial Sequence
  Description of Artificial Sequence Synthetic primer
cgccaagcttgaataggagctttgttcc

SEQ ID NO: 61 - DNA, 30 bp, Artificial Sequence
  Description of Artificial Sequence Synthetic primer
ctaggatccaggagctgttgatcctttagg

SEQ ID NO: 62 - DNA, 23 bp, Artificial Sequence
  Description of Artificial Sequence Synthetic oligonucleotide
gtggactttggatggtgagatag

SEQ ID NO: 63 - DNA, 23 bp, Artificial Sequence
  Description of Artificial Sequence Synthetic oligonucleotide
gcctggcaagagtgaactgagtc

SEQ ID NO: 64 - DNA, 23 bp, Artificial Sequence
  Description of Artificial Sequence Synthetic oligonucleotide
aagataatgagttgtggcagagc

SEQ ID NO: 65 - DNA, 24 bp, Artificial Sequence
  Description of Artificial Sequence Synthetic oligonucleotide
tctacctggtaatccagcatctgg

SEQ ID NO: 66 - DNA, 23 bp, Artificial Sequence
  Description of Artificial Sequence Synthetic oligonucleotide
ataggaggaaggcaccaagaggg

SEQ ID NO: 67 - DNA, 23 bp, Artificial Sequence
  Description of Artificial Sequence Synthetic oligonucleotide
aatgatgctttggtcctactcct

SEQ ID NO: 68 - DNA, 24 bp, Artificial Sequence
  Description of Artificial Sequence Synthetic oligonucleotide
tgctcttgctactctggcatgtac

SEQ ID NO: 69 - DNA, 23 bp, Artificial Sequence
  Description of Artificial Sequence Synthetic oligonucleotide
aatctacctctgagagctgcagg

SEQ ID NO: 70 - DNA, 23 bp, Artificial Sequence
  Description of Artificial Sequence Synthetic oligonucleotide
tcagacacagctgaagcagaggc

SEQ ID NO: 71 - DNA, 23 bp, Artificial Sequence
  Description of Artificial Sequence Synthetic oligonucleotide
atgccagtgtcagtagatgtcag

SEQ ID NO: 72 - DNA, 24 bp, Artificial Sequence
  Description of Artificial Sequence Synthetic oligonucleotide
tcaagatcagccagagtgcacatg

SEQ ID NO: 73 - DNA, 23 bp, Artificial Sequence
  Description of Artificial Sequence Synthetic oligonucleotide
tgctcttccgagcctctctggag

SEQ ID NO: 74 - DNA, 22 bp, Artificial Sequence
  Description of Artificial Sequence Synthetic oligonucleotide
atggactatcatatgcttaccg

SEQ ID NO: 75 - DNA, 28 bp, Artificial Sequence
  Description of Artificial Sequence Synthetic oligonucleotide
gcttcagcaagccgagtcctgcgtcgag

SEQ ID NO: 76 - DNA, 28 bp, Artificial Sequence
  Description of Artificial Sequence Synthetic oligonucleotide
gctcctctggtttccctttcgctttcaa

SEQ ID NO: 77 - DNA, 22 bp, Artificial Sequence
  Description of Artificial Sequence Synthetic oligonucleotide
gtaatacgactcactatagggc

SEQ ID NO: 78 - DNA, 19 bp, Artificial Sequence
  Description of Artificial Sequence Synthetic oligonucleotide
actatagggcacgcgtggt

SEQ ID NO: 79 - DNA, 23 bp, Artificial Sequence
  Description of Artificial Sequence Synthetic oligonucleotide
tcagacccttttagtcagtgtgg

SEQ ID NO: 80 - DNA, 23 bp, Artificial Sequence
  Description of Artificial Sequence Synthetic oligonucleotide
ttgcttgtactgggtctctctgg

SEQ ID NO: 81 - DNA, 23 bp, Artificial Sequence
  Description of Artificial Sequence Synthetic oligonucleotide
cagctgctttttgcttgtactgg

SEQ ID NO: 82 - DNA, 23 bp, Artificial Sequence
  Description of Artificial Sequence Synthetic oligonucleotide
ctgacatcgagcttgctacaagg

SEQ ID NO: 83 - DNA, 23 bp, Artificial Sequence
  Description of Artificial Sequence Synthetic oligonucleotide
ccgcctagcatttcatcacatgg

SEQ ID NO: 84 - DNA, 23 bp, Artificial Sequence
  Description of Artificial Sequence Synthetic oligonucleotide
cggagagagaagtattagagtgg

SEQ ID NO: 85 - DNA, 23 bp, Artificial Sequence
  Description of Artificial Sequence Synthetic oligonucleotide
agtaccagttgagcaagagaagg

SEQ ID NO: 86 - DNA, 23 bp, Artificial Sequence
  Description of Artificial Sequence Synthetic oligonucleotide
gatatccactgacctttggatgg

SEQ ID NO: 87 - DNA, 23 bp, Artificial Sequence
  Description of Artificial Sequence Synthetic oligonucleotide
gattggcagaactacacaccagg

SEQ ID NO: 88 - DNA, 23 bp, Artificial Sequence
  Description of Artificial Sequence Synthetic oligonucleotide
cacaaggctacttccctgattgg

SEQ ID NO: 89 - DNA, 23 bp, Artificial Sequence
  Description of Artificial Sequence Synthetic oligonucleotide
ctgtggatctaccacacacaagg

SEQ ID NO: 90 - DNA, 23 bp, Artificial Sequence
  Description of Artificial Sequence Synthetic oligonucleotide
tgggagctctctggctaactagg

SEQ ID NO: 91 - DNA, 23 bp, Artificial Sequence
  Description of Artificial Sequence Synthetic oligonucleotide
ggttagaccagatctgagcctgg

SEQ ID NO: 92 - DNA, 23 bp, Artificial Sequence
  Description of Artificial Sequence Synthetic oligonucleotide
tgctacaagggactttccgctgg

SEQ ID NO: 93 - DNA, 23 bp, Artificial Sequence
  Description of Artificial Sequence Synthetic oligonucleotide
agagagaagtattagagtggagg

SEQ ID NO: 94 - DNA, 23 bp, Artificial Sequence
  Description of Artificial Sequence Synthetic oligonucleotide
ttacaccctgtgagcctgcatgg

SEQ ID NO: 95 - DNA, 23 bp, Artificial Sequence
  Description of Artificial Sequence Synthetic oligonucleotide
aaggtagaagaagccaatgaagg

SEQ ID NO: 96 - DNA, 23 bp, Artificial Sequence
  Description of Artificial Sequence Synthetic oligonucleotide
atcagatatccactgacctttgg

SEQ ID NO: 97 - DNA, 23 bp, Artificial Sequence
  Description of Artificial Sequence Synthetic oligonucleotide
gacaagatatccttgatctgtgg

SEQ ID NO: 98 - DNA, 23 bp, Artificial Sequence
  Description of Artificial Sequence Synthetic oligonucleotide
gcccgtctgttgtgtgactctgg

SEQ ID NO: 99 - DNA, 23 bp, Artificial Sequence
  Description of Artificial Sequence Synthetic oligonucleotide
atctgagcctgggagctctctgg

SEQ ID NO: 100 - DNA, 23 bp, Artificial Sequence
  Description of Artificial Sequence Synthetic oligonucleotide
ctttccgctggggactttccagg

SEQ ID NO: 101 - DNA, 23 bp, Artificial Sequence
  Description of Artificial Sequence Synthetic oligonucleotide
cagaactacacaccagggccagg

SEQ ID NO: 102 - DNA, 23 bp, Artificial Sequence
  Description of Artificial Sequence Synthetic oligonucleotide
cctgcatgggatggatgacccgg

SEQ ID NO: 103 - DNA, 23 bp, Artificial Sequence
  Description of Artificial Sequence Synthetic oligonucleotide
ccctgtgagcctgcatgggatgg

SEQ ID NO: 104 - DNA, 23 bp, Artificial Sequence
  Description of Artificial Sequence Synthetic oligonucleotide
ctttccagggaggcgtggcctgg

SEQ ID NO: 105 - DNA, 23 bp, Artificial Sequence
  Description of Artificial Sequence Synthetic oligonucleotide
ggggactttccagggaggcgtgg

SEQ ID NO: 106 - DNA, 23 bp, Artificial Sequence
  Description of Artificial Sequence Synthetic oligonucleotide
ccgctggggactttccagggagg

SEQ ID NO: 107 - DNA, 23 bp, Artificial Sequence
  Description of Artificial Sequence Synthetic oligonucleotide
catggcccgagagctgcatccgg

SEQ ID NO: 108 - DNA, 23 bp, Artificial Sequence
  Description of Artificial Sequence Synthetic oligonucleotide
gcctgggcgggactggggagtgg

SEQ ID NO: 109 - DNA, 23 bp, Artificial Sequence
  Description of Artificial Sequence Synthetic oligonucleotide
aggcgtggcctgggcgggactgg

SEQ ID NO: 110 - DNA, 23 bp, Artificial Sequence
  Description of Artificial Sequence Synthetic oligonucleotide
gcgtggcctgggcgggactgggg

SEQ ID NO: 111 - DNA, 23 bp, Artificial Sequence
  Description of Artificial Sequence Synthetic oligonucleotide
ccagggaggcgtggcctgggcgg

SEQ ID NO: 112 - DNA, 23 bp, Artificial Sequence
  Description of Artificial Sequence Synthetic oligonucleotide
tgtggtagatccacagatcaagg

SEQ ID NO: 113 - DNA, 23 bp, Artificial Sequence
  Description of Artificial Sequence Synthetic oligonucleotide
ggtgtgtagttctgccaatcagg

SEQ ID NO: 114 - DNA, 23 bp, Artificial Sequence
  Description of Artificial Sequence Synthetic oligonucleotide
gtcagtggatatctgatccctgg

SEQ ID NO: 115 - DNA, 23 bp, Artificial Sequence
  Description of Artificial Sequence Synthetic oligonucleotide
tagcaccatccaaaggtcagtgg

SEQ ID NO: 116 - DNA, 23 bp, Artificial Sequence
  Description of Artificial Sequence Synthetic oligonucleotide
tagcttgtagcaccatccaaagg

SEQ ID NO: 117 - DNA, 23 bp, Artificial Sequence
  Description of Artificial Sequence Synthetic oligonucleotide
tctaccttctcttgctcaactgg

SEQ ID NO: 118 - DNA, 23 bp, Artificial Sequence
  Description of Artificial Sequence Synthetic oligonucleotide
cactctaatacttctctctccgg

SEQ ID NO: 119 - DNA, 23 bp, Artificial Sequence
  Description of Artificial Sequence Synthetic oligonucleotide
ccatgtgatgaaatgctaggcgg

SEQ ID NO: 120 - DNA, 23 bp, Artificial Sequence
  Description of Artificial Sequence Synthetic oligonucleotide
gggccatgtgatgaaatgctagg

SEQ ID NO: 121 - DNA, 23 bp, Artificial Sequence
  Description of Artificial Sequence Synthetic oligonucleotide
cagcagttcttgaagtactccgg

SEQ ID NO: 122 - DNA, 23 bp, Artificial Sequence
  Description of Artificial Sequence Synthetic oligonucleotide
ctgcttatatgcagcatctgagg

SEQ ID NO: 123 - DNA, 23 bp, Artificial Sequence
  Description of Artificial Sequence Synthetic oligonucleotide
cacactacttgaagcactcaagg

SEQ ID NO: 124 - DNA, 23 bp, Artificial Sequence
  Description of Artificial Sequence Synthetic oligonucleotide
taccagagtcacacaacagacgg

SEQ ID NO: 125 - DNA, 23 bp, Artificial Sequence
  Description of Artificial Sequence Synthetic oligonucleotide
acactgactaaaagggtctgagg

SEQ ID NO: 126 - DNA, 23 bp, Artificial Sequence
  Description of Artificial Sequence Synthetic oligonucleotide
caaggatatcttgtcttcgttgg

SEQ ID NO: 127 - DNA, 23 bp, Artificial Sequence
  Description of Artificial Sequence Synthetic oligonucleotide
cagggaagtagccttgtgtgtgg

SEQ ID NO: 128 - DNA, 23 bp, Artificial Sequence
  Description of Artificial Sequence Synthetic oligonucleotide
gcgggtgttctctccttcattgg

SEQ ID NO: 129 - DNA, 23 bp, Artificial Sequence
  Description of Artificial Sequence Synthetic oligonucleotide
tagttagccagagagctcccagg

SEQ ID NO: 130 - DNA, 23 bp, Artificial Sequence
  Description of Artificial Sequence Synthetic oligonucleotide
ctttattgaggcttaagcagtgg

SEQ ID NO: 131 - DNA, 23 bp, Artificial Sequence
  Description of Artificial Sequence Synthetic oligonucleotide
actcaaggcaagctttattgagg

SEQ ID NO: 132 - DNA, 23 bp, Artificial Sequence
  Description of Artificial Sequence Synthetic oligonucleotide
ggatatctgatccctggccctgg

SEQ ID NO: 133 - DNA, 23 bp, Artificial Sequence
  Description of Artificial Sequence Synthetic oligonucleotide
ggctcacagggtgtaacaagcgg

SEQ ID NO: 134 - DNA, 23 bp, Artificial Sequence
  Description of Artificial Sequence Synthetic oligonucleotide
tccatcccatgcaggctcacagg

SEQ ID NO: 135 - DNA, 23 bp, Artificial Sequence
  Description of Artificial Sequence Synthetic oligonucleotide
agtactccggatgcagctctcgg

SEQ ID NO: 136 - DNA, 23 bp, Artificial Sequence
  Description of Artificial Sequence Synthetic oligonucleotide
agagctcccaggctcagatctgg

SEQ ID NO: 137 - DNA, 23 bp, Artificial Sequence
  Description of Artificial Sequence Synthetic oligonucleotide
gattttccacactgactaaaagg

SEQ ID NO: 138 - DNA, 23 bp, Artificial Sequence
  Description of Artificial Sequence Synthetic oligonucleotide
ccgggtcatccatcccatgcagg

SEQ ID NO: 139 - DNA, 23 bp, Artificial Sequence
  Description of Artificial Sequence Synthetic oligonucleotide
cctccctggaaagtccccagcgg

SEQ ID NO: 140 - DNA, 23 bp, Artificial Sequence
  Description of Artificial Sequence Synthetic oligonucleotide
gccactccccagtcccgcccagg

SEQ ID NO: 141 - DNA, 23 bp, Artificial Sequence
  Description of Artificial Sequence Synthetic oligonucleotide
ccgcccaggccacgcctccctgg

SEQ ID NO: 142 - DNA, 23 bp, Human immunodeficiency virus 1
atcagatatccactgacctttgg

SEQ ID NO: 143 - DNA, 22 bp, Human immunodeficiency virus 1
tcagatatccactgacctttgg

SEQ ID NO: 144 - DNA, 22 bp, Human immunodeficiency virus 1
tcagatatccactgacctttgg

SEQ ID NO: 145 - DNA, 21 bp, Human immunodeficiency virus 1
cagatatccactgacctttgg

SEQ ID NO: 146 - DNA, 21 bp, Human immunodeficiency virus 1
cagatatccactgacctttgg

SEQ ID NO: 147 - DNA, 20 bp, Human immunodeficiency virus 1
agatatccactgacctttgg

SEQ ID NO: 148 - DNA, 20 bp, Human immunodeficiency virus 1
agatatccactgacctttgg

SEQ ID NO: 149 - DNA, 19 bp, Human immunodeficiency virus 1
gatatccactgacctttgg

SEQ ID NO: 150 - DNA, 19 bp, Human immunodeficiency virus 1
gatatccactgacctttgg

SEQ ID NO: 151 - DNA, 18 bp, Human immunodeficiency virus 1
atatccactgacctttgg

SEQ ID NO: 152 - DNA, 18 bp, Human immunodeficiency virus 1
atatccactgacctttgg

SEQ ID NO: 153 - DNA, 17 bp, Human immunodeficiency virus 1
tatccactgaccttggg

SEQ ID NO: 154 - DNA, 17 bp, Human immunodeficiency virus 1
tatccactgacctttgg

SEQ ID NO: 155 - DNA, 17 bp, Human immunodeficiency virus 1
tatccactgacctttgg

SEQ ID NO: 156 - DNA, 17 bp, Human immunodeficiency virus 1
tatccactgaccttaag

SEQ ID NO: 157 - DNA, 17 bp, Human immunodeficiency virus 1
tatccactgaccttgag

SEQ ID NO: 158 - DNA, 16 bp, Human immunodeficiency virus 1
atccactgaccttagg

SEQ ID NO: 159 - DNA, 16 bp, Human immunodeficiency virus 1
atccactgaccttagg

SEQ ID NO: 160 - DNA, 16 bp, Human immunodeficiency virus 1
atccactgaccttggg

SEQ ID NO: 161 - DNA, 16 bp, Human immunodeficiency virus 1
atccactgaccttggg

SEQ ID NO: 162 - DNA, 16 bp, Human immunodeficiency virus 1
atccactgaccttggg

SEQ ID NO: 163 - DNA, 16 bp, Human immunodeficiency virus 1
atccactgaccttggg

SEQ ID NO: 164 - DNA, 16 bp, Human immunodeficiency virus 1
atccactgacctttgg

SEQ ID NO: 165 - DNA, 16 bp, Human immunodeficiency virus 1
atccactgacctttgg

SEQ ID NO: 166 - DNA, 16 bp, Human immunodeficiency virus 1
atccactgacctttgg

SEQ ID NO: 167 - DNA, 16 bp, Human immunodeficiency virus 1
atccactgaccttaag

SEQ ID NO: 168 - DNA, 16 bp, Human immunodeficiency virus 1
atccactgaccttaag

SEQ ID NO: 169 - DNA, 16 bp, Human immunodeficiency virus 1
atccactgaccttcag

SEQ ID NO: 170 - DNA, 16 bp, Human immunodeficiency virus 1
atccactgaccttcag

SEQ ID NO: 171 - DNA, 16 bp, Human immunodeficiency virus 1
atccactgaccttgag

SEQ ID NO: 172 - DNA, 16 bp, Human immunodeficiency virus 1
atccactgaccttgag

SEQ ID NO: 173 - DNA, 15 bp, Human immunodeficiency virus 1
tccactgaccttagg

SEQ ID NO: 174 - DNA, 15 bp, Human immunodeficiency virus 1
tccactgaccttagg

SEQ ID NO: 175 - DNA, 15 bp, Human immunodeficiency virus 1
tccactgaccttagg

SEQ ID NO: 176 - DNA, 15 bp, Human immunodeficiency virus 1
tccactgaccttagg

SEQ ID NO: 177 - DNA, 15 bp, Human immunodeficiency virus 1
tccactgaccttagg

SEQ ID NO: 178 - DNA, 15 bp, Human immunodeficiency virus 1
tccactgaccttagg

SEQ ID NO: 179 - DNA, 15 bp, Human immunodeficiency virus 1
tccactgaccttggg

SEQ ID NO: 180 - DNA, 15 bp, Human immunodeficiency virus 1
tccactgaccttggg

SEQ ID NO: 181 - DNA, 15 bp, Human immunodeficiency virus 1
tccactgaccttggg

SEQ ID NO: 182 - DNA, 15 bp, Human immunodeficiency virus 1
tccactgaccttggg

SEQ ID NO: 183 - DNA, 15 bp, Human immunodeficiency virus 1
tccactgaccttggg

SEQ ID NO: 184 - DNA, 15 bp, Human immunodeficiency virus 1
tccactgaccttggg

SEQ ID NO: 185 - DNA, 15 bp, Human immunodeficiency virus 1
tccactgaccttggg

SEQ ID NO: 186 - DNA, 15 bp, Human immunodeficiency virus 1
tccactgaccttggg

SEQ ID NO: 187 - DNA, 15 bp, Human immunodeficiency virus 1
tccactgacctttgg

SEQ ID NO: 188 - DNA, 15 bp, Human immunodeficiency virus 1
tccactgacctttgg

SEQ ID NO: 189 - DNA, 15 bp, Human immunodeficiency virus 1
tccactgacctttgg

SEQ ID NO: 190 - DNA, 15 bp, Human immunodeficiency virus 1
tccactgacctttgg

SEQ ID NO: 191 - DNA, 15 bp, Human immunodeficiency virus 1
tccactgacctttgg

SEQ ID NO: 192 - DNA, 15 bp, Human immunodeficiency virus 1
tccactgacctttgg

SEQ ID NO: 193 - DNA, 15 bp, Human immunodeficiency virus 1
tccactgacctttgg

SEQ ID NO: 194 - DNA, 15 bp, Human immunodeficiency virus 1
tccactgacctttgg

SEQ ID NO: 195 - DNA, 15 bp, Human immunodeficiency virus 1
tccactgacctttgg

SEQ ID NO: 196 - DNA, 15 bp, Human immunodeficiency virus 1
tccactgaccttaag

SEQ ID NO: 197 - DNA, 15 bp, Human immunodeficiency virus 1
tccactgaccttaag

SEQ ID NO: 198 - DNA, 15 bp, Human immunodeficiency virus 1
tccactgaccttaag

SEQ ID NO: 199 - DNA, 15 bp, Human immunodeficiency virus 1
tccactgaccttaag

SEQ ID NO: 200 - DNA, 15 bp, Human immunodeficiency virus 1
tccactgaccttaag

SEQ ID NO: 201 - DNA, 15 bp, Human immunodeficiency virus 1
tccactgaccttcag

SEQ ID NO: 202 - DNA, 15 bp, Human immunodeficiency virus 1
tccactgaccttcag

SEQ ID NO: 203 - DNA, 15 bp, Human immunodeficiency virus 1
tccactgaccttcag

SEQ ID NO: 204 - DNA, 15 bp, Human immunodeficiency virus 1
tccactgaccttcag

SEQ ID NO: 205 - DNA, 15 bp, Human immunodeficiency virus 1
tccactgaccttcag

SEQ ID NO: 206 - DNA, 15 bp, Human immunodeficiency virus 1
tccactgaccttcag

SEQ ID NO: 207 - DNA, 15 bp, Human immunodeficiency virus 1
tccactgaccttcag

SEQ ID NO: 208 - DNA, 15 bp, Human immunodeficiency virus 1
tccactgaccttcag

SEQ ID NO: 209 - DNA, 15 bp, Human immunodeficiency virus 1
tccactgaccttcag

SEQ ID NO: 210 - DNA, 15 bp, Human immunodeficiency virus 1
tccactgaccttcag

SEQ ID NO: 211 - DNA, 15 bp, Human immunodeficiency virus 1
tccactgaccttcag

SEQ ID NO: 212 - DNA, 15 bp, Human immunodeficiency virus 1
tccactgaccttcag

SEQ ID NO: 213 - DNA, 15 bp, Human immunodeficiency virus 1
tccactgaccttgag

SEQ ID NO: 214 - DNA, 15 bp, Human immunodeficiency virus 1
tccactgaccttgag

SEQ ID NO: 215 - DNA, 15 bp, Human immunodeficiency virus 1
tccactgaccttgag

SEQ ID NO: 216 - DNA, 15 bp, Human immunodeficiency virus 1
tccactgaccttgag

SEQ ID NO: 217 - DNA, 15 bp, Human immunodeficiency virus 1
tccactgaccttgag

SEQ ID NO: 218 - DNA, 15 bp, Human immunodeficiency virus 1
tccactgaccttgag

SEQ ID NO: 219 - DNA, 15 bp, Human immunodeficiency virus 1
tccactgaccttgag

SEQ ID NO: 220 - DNA, 15 bp, Human immunodeficiency virus 1
tccactgaccttgag

SEQ ID NO: 221 - DNA, 15 bp, Human immunodeficiency virus 1
tccactgaccttgag

SEQ ID NO: 222 - DNA, 15 bp, Human immunodeficiency virus 1
tccactgacctttag

SEQ ID NO: 223 - DNA, 15 bp, Human immunodeficiency virus 1
tccactgacctttag

SEQ ID NO: 224 - DNA, 15 bp, Human immunodeficiency virus 1
tccactgacctttag

SEQ ID NO: 225 - DNA, 15 bp, Human immunodeficiency virus 1
tccactgacctttag

SEQ ID NO: 226 - DNA, 15 bp, Human immunodeficiency virus 1
tccactgacctttag

SEQ ID NO: 227 - DNA, 23 bp, Human immunodeficiency virus 1
cagcagttcttgaagtactccgg

SEQ ID NO: 228 - DNA, 22 bp, Human immunodeficiency virus 1
agcagttcttgaagtactccgg

SEQ ID NO: 229 - DNA, 21 bp, Human immunodeficiency virus 1
gcagttcttgaagtactccgg

SEQ ID NO: 230 - DNA, 20 bp, Human immunodeficiency virus 1
cagttcttgaagtactccgg

SEQ ID NO: 231 - DNA, 19 bp, Human immunodeficiency virus 1
agttcttgaagtactccgg

SEQ ID NO: 232 - DNA, 18 bp, Human immunodeficiency virus 1
gttcttgaagtactccgg

SEQ ID NO: 233 - DNA, 17 bp, Human immunodeficiency virus 1
ttcttgaagtactccgg

SEQ ID NO: 234 - DNA, 16 bp, Human immunodeficiency virus 1
tcttgaagtactccgg

SEQ ID NO: 235 - DNA, 16 bp, Human immunodeficiency virus 1
tcttgaagtactctag

SEQ ID NO: 236 - DNA, 15 bp, Human immunodeficiency virus 1
cttgaagtactcagg

SEQ ID NO: 237 - DNA, 15 bp, Human immunodeficiency virus 1
cttgaagtactcagg

SEQ ID NO: 238 - DNA, 15 bp, Human immunodeficiency virus 1
cttgaagtactcagg

SEQ ID NO: 239 - DNA, 15 bp, Human immunodeficiency virus 1
cttgaagtactcagg

SEQ ID NO: 240 - DNA, 15 bp, Human immunodeficiency virus 1
cttgaagtactccgg

SEQ ID NO: 241 - DNA, 15 bp, Human immunodeficiency virus 1
cttgaagtactctgg

SEQ ID NO: 242 - DNA, 15 bp, Human immunodeficiency virus 1
cttgaagtactcaag

SEQ ID NO: 243 - DNA, 15 bp, Human immunodeficiency virus 1
cttgaagtactcaag

SEQ ID NO: 244 - DNA, 15 bp, Human immunodeficiency virus 1
cttgaagtactcaag

SEQ ID NO: 245 - DNA, 15 bp, Human immunodeficiency virus 1
cttgaagtactcaag

SEQ ID NO: 246 - DNA, 15 bp, Human immunodeficiency virus 1
cttgaagtactcaag

SEQ ID NO: 247 - DNA, 15 bp, Human immunodeficiency virus 1
cttgaagtactccag

SEQ ID NO: 248 - DNA, 15 bp, Human immunodeficiency virus 1
cttgaagtactccag

SEQ ID NO: 249 - DNA, 15 bp, Human immunodeficiency virus 1
cttgaagtactccag

SEQ ID NO: 250 - DNA, 15 bp, Human immunodeficiency virus 1
cttgaagtactccag

SEQ ID NO: 251 - DNA, 15 bp, Human immunodeficiency virus 1
cttgaagtactctag

SEQ ID NO: 252 - DNA, 15 bp, Human immunodeficiency virus 1
cttgaagtactctag

SEQ ID NO: 253 - DNA, 23 bp, Human immunodeficiency virus 1
atcagatatccactgacctttgg

SEQ ID NO: 254 - DNA, 22 bp, Human immunodeficiency virus 1
tcagatatccactgacctttgg

SEQ ID NO: 255 - DNA, 22 bp, Human immunodeficiency virus 1
tcagatatccactgacctttgg

SEQ ID NO: 256 - DNA, 21 bp, Human immunodeficiency virus 1
cagatatccactgacctttgg

SEQ ID NO: 257 - DNA, 21 bp, Human immunodeficiency virus 1
cagatatccactgacctttgg

SEQ ID NO: 258 - DNA, 20 bp, Human immunodeficiency virus 1
agatatccactgacctttgg

SEQ ID NO: 259 - DNA, 20 bp, Human immunodeficiency virus 1
agatatccactgacctttgg

SEQ ID NO: 260 - DNA, 19 bp, Human immunodeficiency virus 1
gatatccactgacctttgg

SEQ ID NO: 261 - DNA, 19 bp, Human immunodeficiency virus 1
gatatccactgacctttgg

SEQ ID NO: 262 - DNA, 18 bp, Human immunodeficiency virus 1
atatccactgacctttgg

SEQ ID NO: 263 - DNA, 18 bp, Human immunodeficiency virus 1
atatccactgacctttgg

SEQ ID NO: 264 - DNA, 17 bp, Human immunodeficiency virus 1
tatccactgaccttggg

SEQ ID NO: 265 - DNA, 17 bp, Human immunodeficiency virus 1
tatccactgacctttgg

SEQ ID NO: 266 - DNA, 17 bp, Human immunodeficiency virus 1
tatccactgacctttgg

SEQ ID NO: 267 - DNA, 17 bp, Human immunodeficiency virus 1
tatccactgaccttaag

SEQ ID NO: 268 - DNA, 17 bp, Human immunodeficiency virus 1
tatccactgaccttgag

SEQ ID NO: 269 - DNA, 16 bp, Human immunodeficiency virus 1
atccactgaccttagg

SEQ ID NO: 270 - DNA, 16 bp, Human immunodeficiency virus 1
atccactgaccttagg

SEQ ID NO: 271 - DNA, 16 bp, Human immunodeficiency virus 1
atccactgaccttggg

SEQ ID NO: 272 - DNA, 16 bp, Human immunodeficiency virus 1
atccactgaccttggg

SEQ ID NO: 273 - DNA, 16 bp, Human immunodeficiency virus 1
atccactgaccttggg

SEQ ID NO: 274 - DNA, 16 bp, Human immunodeficiency virus 1
atccactgaccttggg

SEQ ID NO: 275 - DNA, 16 bp, Human immunodeficiency virus 1
atccactgacctttgg

SEQ ID NO: 276 - DNA, 16 bp, Human immunodeficiency virus 1
atccactgacctttgg

SEQ ID NO: 277 - DNA, 16 bp, Human immunodeficiency virus 1
atccactgacctttgg

SEQ ID NO: 278 - DNA, 16 bp, Human immunodeficiency virus 1
atccactgaccttaag

SEQ ID NO: 279 - DNA, 16 bp, Human immunodeficiency virus 1
atccactgaccttaag

SEQ ID NO: 280 - DNA, 16 bp, Human immunodeficiency virus 1
atccactgaccttcag

SEQ ID NO: 281 - DNA, 16 bp, Human immunodeficiency virus 1
atccactgaccttcag

SEQ ID NO: 282 - DNA, 16 bp, Human immunodeficiency virus 1
atccactgaccttgag

SEQ ID NO: 283 - DNA, 16 bp, Human immunodeficiency virus 1
atccactgaccttgag

SEQ ID NO: 284 - DNA, 15 bp, Human immunodeficiency virus 1
tccactgaccttagg

SEQ ID NO: 285 - DNA, 15 bp, Human immunodeficiency virus 1
tccactgaccttagg

SEQ ID NO: 286 - DNA, 15 bp, Human immunodeficiency virus 1
tccactgaccttagg

SEQ ID NO: 287 - DNA, 15 bp, Human immunodeficiency virus 1
tccactgaccttagg

SEQ ID NO: 288 - DNA, 15 bp, Human immunodeficiency virus 1
tccactgaccttagg

SEQ ID NO: 289 - DNA, 15 bp, Human immunodeficiency virus 1
tccactgaccttagg

SEQ ID NO: 290 - DNA, 15 bp, Human immunodeficiency virus 1
tccactgaccttggg

SEQ ID NO: 291 - DNA, 15 bp, Human immunodeficiency virus 1
tccactgaccttggg

SEQ ID NO: 292 - DNA, 15 bp, Human immunodeficiency virus 1
tccactgaccttggg

SEQ ID NO: 293 - DNA, 15 bp, Human immunodeficiency virus 1
tccactgaccttggg

SEQ ID NO: 294 - DNA, 15 bp, Human immunodeficiency virus 1
tccactgaccttggg

SEQ ID NO: 295 - DNA, 15 bp, Human immunodeficiency virus 1
tccactgaccttggg

SEQ ID NO: 296 - DNA, 15 bp, Human immunodeficiency virus 1
tccactgaccttggg

SEQ ID NO: 297 - DNA, 15 bp, Human immunodeficiency virus 1
tccactgaccttggg

SEQ ID NO: 298 - DNA, 15 bp, Human immunodeficiency virus 1
tccactgacctttgg

SEQ ID NO: 299 - DNA, 15 bp, Human immunodeficiency virus 1
tccactgacctttgg

SEQ ID NO: 300 - DNA, 15 bp, Human immunodeficiency virus 1
tccactgacctttgg

SEQ ID NO: 301 - DNA, 15 bp, Human immunodeficiency virus 1
tccactgacctttgg

SEQ ID NO: 302 - DNA, 15 bp, Human immunodeficiency virus 1
tccactgacctttgg

SEQ ID NO: 303 - DNA, 15 bp, Human immunodeficiency virus 1
tccactgacctttgg

SEQ ID NO: 304 - DNA, 15 bp, Human immunodeficiency virus 1
tccactgacctttgg

SEQ ID NO: 305 - DNA, 15 bp, Human immunodeficiency virus 1
tccactgacctttgg

SEQ ID NO: 306 - DNA, 15 bp, Human immunodeficiency virus 1
tccactgacctttgg

SEQ ID NO: 307 - DNA, 15 bp, Human immunodeficiency virus 1
tccactgaccttaag

SEQ ID NO: 308 - DNA, 15 bp, Human immunodeficiency virus 1
tccactgaccttaag

SEQ ID NO: 309 - DNA, 15 bp, Human immunodeficiency virus 1
tccactgaccttaag

SEQ ID NO: 310 - DNA, 15 bp, Human immunodeficiency virus 1
tccactgaccttaag

SEQ ID NO: 311 - DNA, 15 bp, Human immunodeficiency virus 1
tccactgaccttaag

SEQ ID NO: 312 - DNA, 15 bp, Human immunodeficiency virus 1
tccactgaccttcag

SEQ ID NO: 313 - DNA, 15 bp, Human immunodeficiency virus 1
tccactgaccttcag

SEQ ID NO: 314 - DNA, 15 bp, Human immunodeficiency virus 1
tccactgaccttcag

SEQ ID NO: 315 - DNA, 15 bp, Human immunodeficiency virus 1
tccactgaccttcag

SEQ ID NO: 316 - DNA, 15 bp, Human immunodeficiency virus 1
tccactgaccttcag

SEQ ID NO: 317 - DNA, 15 bp, Human immunodeficiency virus 1
tccactgaccttcag

SEQ ID NO: 318 - DNA, 15 bp, Human immunodeficiency virus 1
tccactgaccttcag

SEQ ID NO: 319 - DNA, 15 bp, Human immunodeficiency virus 1
tccactgaccttcag

SEQ ID NO: 320 - DNA, 15 bp, Human immunodeficiency virus 1
tccactgaccttcag

SEQ ID NO: 321 - DNA, 15 bp, Human immunodeficiency virus 1
tccactgaccttcag

SEQ ID NO: 322 - DNA, 15 bp, Human immunodeficiency virus 1
tccactgaccttcag

SEQ ID NO: 323 - DNA, 15 bp, Human immunodeficiency virus 1
tccactgaccttcag

SEQ ID NO: 324 - DNA, 15 bp, Human immunodeficiency virus 1
tccactgaccttgag

SEQ ID NO: 325 - DNA, 15 bp, Human immunodeficiency virus 1
tccactgaccttgag

SEQ ID NO: 326 - DNA, 15 bp, Human immunodeficiency virus 1
tccactgaccttgag

SEQ ID NO: 327 - DNA, 15 bp, Human immunodeficiency virus 1
tccactgaccttgag

SEQ ID NO: 328 - DNA, 15 bp, Human immunodeficiency virus 1
tccactgaccttgag

SEQ ID NO: 329 - DNA, 15 bp, Human immunodeficiency virus 1
tccactgaccttgag

SEQ ID NO: 330 - DNA, 15 bp, Human immunodeficiency virus 1
tccactgaccttgag

SEQ ID NO: 331 - DNA, 15 bp, Human immunodeficiency virus 1
tccactgaccttgag

SEQ ID NO: 332 - DNA, 15 bp, Human immunodeficiency virus 1
tccactgaccttgag

SEQ ID NO: 333 - DNA, 15 bp, Human immunodeficiency virus 1
tccactgacctttag

SEQ ID NO: 334 - DNA, 15 bp, Human immunodeficiency virus 1
tccactgacctttag

SEQ ID NO: 335 - DNA, 15 bp, Human immunodeficiency virus 1
tccactgacctttag

SEQ ID NO: 336 - DNA, 15 bp, Human immunodeficiency virus 1
tccactgacctttag

SEQ ID NO: 337 - DNA, 15 bp, Human immunodeficiency virus 1
tccactgacctttag

SEQ ID NO: 338 - DNA, 23 bp, Human immunodeficiency virus 1
cagcagttcttgaagtactccgg

SEQ ID NO: 339 - DNA, 22 bp, Human immunodeficiency virus 1
agcagttcttgaagtactccgg

SEQ ID NO: 340 - DNA, 21 bp, Human immunodeficiency virus 1
gcagttcttgaagtactccgg

SEQ ID NO: 341 - DNA, 20 bp, Human immunodeficiency virus 1
cagttcttgaagtactccgg

SEQ ID NO: 342 - DNA, 19 bp, Human immunodeficiency virus 1
agttcttgaagtactccgg

SEQ ID NO: 343 - DNA, 18 bp, Human immunodeficiency virus 1
gttcttgaagtactccgg

SEQ ID NO: 344 - DNA, 17 bp, Human immunodeficiency virus 1
ttcttgaagtactccgg

SEQ ID NO: 345 - DNA, 16 bp, Human immunodeficiency virus 1
tcttgaagtactccgg

SEQ ID NO: 346 - DNA, 16 bp, Human immunodeficiency virus 1
tcttgaagtactctag

SEQ ID NO: 347 - DNA, 15 bp, Human immunodeficiency virus 1
cttgaagtactcagg

SEQ ID NO: 348 - DNA, 15 bp, Human immunodeficiency virus 1
cttgaagtactcagg

SEQ ID NO: 349 - DNA, 15 bp, Human immunodeficiency virus 1
cttgaagtactcagg

SEQ ID NO: 350 - DNA, 15 bp, Human immunodeficiency virus 1
cttgaagtactcagg

SEQ ID NO: 351 - DNA, 15 bp, Human immunodeficiency virus 1
cttgaagtactccgg

SEQ ID NO: 352 - DNA, 15 bp, Human immunodeficiency virus 1
cttgaagtactctgg

SEQ ID NO: 353 - DNA, 15 bp, Human immunodeficiency virus 1
cttgaagtactcaag

SEQ ID NO: 354 - DNA, 15 bp, Human immunodeficiency virus 1
cttgaagtactcaag

SEQ ID NO: 355 - DNA, 15 bp, Human immunodeficiency virus 1
cttgaagtactcaag

SEQ ID NO: 356 - DNA, 15 bp, Human immunodeficiency virus 1
cttgaagtactcaag

SEQ ID NO: 357 - DNA, 15 bp, Human immunodeficiency virus 1
cttgaagtactcaag

SEQ ID NO: 358 - DNA, 15 bp, Human immunodeficiency virus 1
cttgaagtactccag

SEQ ID NO: 359 - DNA, 15 bp, Human immunodeficiency virus 1
cttgaagtactccag

SEQ ID NO: 360 - DNA, 15 bp, Human immunodeficiency virus 1
cttgaagtactccag

SEQ ID NO: 361 - DNA, 15 bp, Human immunodeficiency virus 1
cttgaagtactccag

SEQ ID NO: 362 - DNA, 15 bp, Human immunodeficiency virus 1
cttgaagtactctag

SEQ ID NO: 363 - DNA, 15 bp, Human immunodeficiency virus 1
cttgaagtactctag

SEQ ID NO: 364 - DNA, 23 bp, Human immunodeficiency virus 1
gatctgtggatctaccacacaca

SEQ ID NO: 365 - DNA, 26 bp, Human immunodeficiency virus 1
gatctgtggatctaccacacacaagg

SEQ ID NO: 366 - DNA, 20 bp, Human immunodeficiency virus 1
gattggcagaactacacacc

SEQ ID NO: 367 - DNA, 23 bp, Human immunodeficiency virus 1
gattggcagaactacacaccagg

SEQ ID NO: 368 - DNA, 27 bp, Human immunodeficiency virus 1
gccagggatcagatatccactgacctt

SEQ ID NO: 369 - DNA, 30 bp, Human immunodeficiency virus 1; identical to SEQ ID NO: 1

SEQ ID NO: 370 - DNA, 30 bp, Human immunodeficiency virus 1
gagtacttcaagaactgctgacatcgagct

SEQ ID NO: 371 - DNA, 33 bp, Human immunodeficiency virus 1
ccggagtacttcaagaactgctgacatcgagct

SEQ ID NO: 372 - DNA, 20 bp, Human immunodeficiency virus 1
gcgtggcctgggcgggactg

SEQ ID NO: 373 - DNA, 23 bp, Human immunodeficiency virus 1
gcgtggcctgggcgggactgggg

SEQ ID NO: 374 - DNA, 22 bp, Human immunodeficiency virus 1
tcagatgctgcatataagcagc

SEQ ID NO: 375 - DNA, 25 bp, Human immunodeficiency virus 1
ccctcagatgctgcatataagcagc

SEQ ID NO: 376 - DNA, 634 bp, Artificial Sequence
  Description of Artificial Sequence Synthetic polynucleotide
tggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 377 - DNA, 453 bp, Artificial Sequence
  Description of Artificial Sequence Synthetic polynucleotide
tggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcttgtact

SEQ ID NO: 378 - DNA, 97 bp, Artificial Sequence
  Description of Artificial Sequence Synthetic oligonucleotide
gggtctctctggttagaccagatctgagcctgggagctctctggctaactagggaacccactgcttaagcctcaataaagcttgccttgagtgcttc

SEQ ID NO: 379 - DNA, 84 bp, Artificial Sequence
  Description of Artificial Sequence Synthetic oligonucleotide
aagtagtgtgtgcccgtctgttgtgtgactctggtaactagagatccctcagacccttttagtcagtgtggaaaatctctagca

SEQ ID NO: 380 - DNA, 818 bp, Artificial Sequence
  Description of Artificial Sequence Synthetic polynucleotide
tggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcagattgagccctgggaggttctctccagcactagcaggtagagcctgggtgttccctgctagactctcaccagcacttggccggtgctgggcagagtgactccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 381 - DNA, 517 bp, Artificial Sequence
  Description of Artificial Sequence Synthetic polynucleotide
tggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtatt

SEQ ID NO: 382 - DNA, 176 bp, Artificial Sequence
  Description of Artificial Sequence Synthetic polynucleotide
cagtcgctctgcggagaggctggcagattgagccctgggaggttctctccagcactagcaggtagagcctgggtgttccctgctagactctcaccagcacttggccggtgctgggcagagtgactccacgcttgcttgcttaaagccctcttcaataaagctgccattttagaagt

SEQ ID NO: 383 - DNA, 125 bp, Artificial Sequence
  Description of Artificial Sequence Synthetic polynucleotide
aagctagtgtgtgttcccatctctcctagccgccgcctggtcaactcggtactcaataataagaagaccctggtctgttaggaccctttctgctttgggaaaccgaagcaggaaaatccctagca

SEQ ID NO: 384 - DNA, 14825 bp, Human immunodeficiency virus 1
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 385 - DNA, 10535 bp, Simian immunodeficiency virus
gcatgcacattttaaaggcttttgctaaatatagccaaaagtccttctacaaattttctaagagttctgattcaaagcagtaacaggccttgtctcatcatgaactttggcatttcatctacagctaagtttatatcataaatagttctttacaggcagcaccaacttatacccttatagcatactttactgtgtgaaaattgcatctttcattaagcttactgtaaatttactggctgtcttccttgcaggtttctggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcagattgagccctgggaggttctctccagcactagcaggtagagcctgggtgttccctgctagactctcaccagcacttggccggtgctgggcagagtgactccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagcagattggcgcctgaacagggacttgaaggagagtgagagactcctgagtacggctgagtgaaggcagtaagggcggcaggaaccaaccacgacggagtgctcctataaaggcgcgggtcggtaccagacggcgtgaggagcgggagaggaagaggcctccggttgcaggtaagtgcaacacaaaaaagaaatagctgtcttttatccaggaaggggtaataagatagagtgggagatgggcgtgagaaactccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaacaacagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgatgccaactgctcccccagaggacccagctgtggatctgctaaagaactacatgcagttgggcaagcagcagagagaaaagcagagagaaagcagagagaagccttacaaggaggtgacagaggatttgctgcacctcaattctctctttggaggagaccagtagtcactgctcatattgaaggacagcctgtagaagtattactggatacaggggctgatgattctattgtaacaggaatagagttaggtccacattataccccaaaaatagtaggaggaataggaggttttattaatactaaagaatacaaaaatgtagaaatagaagttttaggcaaaaggattaaagggacaatcatgacaggggacaccccgattaacatttttggtagaaatttgctaacagctctggggatgtctctaaattttcccatagctaaagtagagcctgtaaaagtcgccttaaagccaggaaaggatggaccaaaattgaagcagtggccattatcaaaagaaaagatagttgcattaagagaaatctgtgaaaagatggaaaaggatggtcagttggaggaagctcccccgaccaatccatacaacacccccacatttgctataaagaaaaaggataagaacaaatggagaatgctgatagattttagggaactaaatagggtcactcaggactttacggaagtccaattaggaataccacaccctgcaggactagcaaaaaggaaaagaattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagtcaaaggaactcttgaatagcatagggttttctaccccagaagagaaattccaaaaagatcccccatttcaatggatggggtacgaattgtggccaacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataaaataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagtgagactattagcacatgtaatacagaaaataggaaaggaagcaatagtgatctggggacaggtcccaaaattccacttaccagttgagaaggatgtatgggaacagtggtggacagactattggcaggtaacctggataccggaatgggattttatctcaacaccaccgctagtaagattagtcttcaatctagtgaaggaccctatagagggagaagaaacctattatacagatggatcatgtaataaacagtcaaaagaagggaaagcaggatatatcacagataggggcaaagacaaagtaaaagtgttagaacagactactaatcaacaagcagaattggaagcatttctcatggcattgacagactcagggccaaaggcaaatattatagtagattcacaatatgttatgggaataataacaggatgccctacagaatcagagagcaggctagttaatcaaataatagaagaaatgattaaaaagtcagaaatttatgtagcatgggtaccagcacacaaaggtataggaggaaaccaagaaatagaccacctagttagtcaagggattagacaagttctcttcttggaaaagatagagccagcacaagaagaacatgataaataccatagtaatgtaaaagaattggtattcaaatttggattacccagaatagtggccagacagatagtagacacctgtgataaatgtcatcagaaaggagaggctatacatgggcaggcaaattcagatctagggacttggcaaatggattgtacccatctagagggaaaaataatcatagttgcagtacatgtagctagtggattcatagaagcagaggtaattccacaagagacaggaagacagacagcactatttctgttaaaattggcaggcagatggcctattacacatctacacacagataatggtgctaactttgcttcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagagatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagtaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgatgaacaagggatgtcaccaagctatgtaaaatacagatacttgtgtttaatacaaaaggctttatttatgcattgcaagaaaggctgtagatgtctaggggaaggacatggggcagggggatggagaccaggacctcctcctcctccccctccaggactagcataaatggaagaaagacctccagaaaatgaaggaccacaaagggaaccatgggatgaatgggtagtggaggttctggaagaactgaaagaagaagctttaaaacattttgatcctcgcttgctaactgcacttggtaatcatatctataatagacatggagacacccttgagggagcaggagaactcattagaatcctccaacgagcgctcttcatgcatttcagaggcggatgcatccactccagaatcggccaacctgggggaggaaatcctctctcagctataccgccctctagaagcatgctataacacatgctattgtaaaaagtgttgctaccattgccagttttgttttcttaaaaaaggcttggggatatgttatgagcaatcacgaaagagaagaagaactccgaaaaaggctaaggctaatacatcttctgcatcaaacaagtaagtatgggatgtcttgggaatcagctgcttatcgccatcttgcttttaagtgtctatgggatctattgtactctatatgtcacagtcttttatggtgtaccagcttggaggaatgcgacaattcccctcttttgtgcaaccaagaatagggatacttggggaacaactcagtgcctaccagataatggtgattattcagaagtggcccttaatgttacagaaagctttgatgcctggaataatacagtcacagaacaggcaatagaggatgtatggcaactctttgagacctcaataaagccttgtgtaaaattatccccattatgcattactatgagatgcaataaaagtgagacagatagatggggattgacaaaatcaataacaacaacagcatcaacaacatcaacgacagcatcagcaaaagtagacatggtcaatgagactagttcttgtatagcccaggataattgcacaggcttggaacaagagcaaatgataagctgtaaattcaacatgacagggttaaaaagagacaagaaaaaagagtacaatgaaacttggtactctgcagatttggtatgtgaacaagggaataacactggtaatgaaagtagatgttacatgaaccactgtaacacttctgttatccaagagtcttgtgacaaacattattgggatgctattagatttaggtattgtgcacctccaggttatgctttgcttagatgtaatgacacaaattattcaggctttatgcctaaatgttctaaggtggtggtctcttcatgcacaaggatgatggagacacagacttctacttggtttggctttaatggaactagagcagaaaatagaacttatatttactggcatggtagggataataggactataattagtttaaataagtattataatctaacaatgaaatgtagaagaccaggaaataagacagttttaccagtcaccattatgtctggattggttttccactcacaaccaatcaatgataggccaaagcaggcatggtgttggtttggaggaaaatggaaggatgcaataaaagaggtgaagcagaccattgtcaaacatcccaggtatactggaactaacaatactgataaaatcaatttgacggctcctggaggaggagatccggaagttaccttcatgtggacaaattgcagaggagagttcctctactgtaaaatgaattggtttctaaattgggtagaagataggaatacagctaaccagaagccaaaggaacagcataaaaggaattacgtgccatgtcatattagacaaataatcaacacttggcataaagtaggcaaaaatgtttatttgcctccaagagagggagacctcacgtgtaactccacagtgaccagtctcatagcaaacatagattggattgatggaaaccaaactaatatcaccatgagtgcagaggtggcagaactgtatcgattggaattgggagattataaattagtagagatcactccaattggcttggcccccacagatgtgaagaggtacactactggtggcacctcaagaaataaaagaggggtctttgtgctagggttcttgggttttctcgcaacggcaggttctgcaatgggcgcggcgtcgttgacgctgaccgctcagtcccgaactttattggctgggatagtgcagcaacagcaacagctgttggacgtggtcaagagacaacaagaattgttgcgactgaccgtctggggaacaaagaacctccagactagggtcactgccatcgagaagtacttaaaggaccaggcgcagctgaatgcttggggatgtgcgtttagacaagtctgccacactactgtaccatggccaaatgcaagtctaacaccaaagtggaacaatgagacttggcaagagtgggagcgaaaggttgacttcttggaagaaaatataacagccctcctagaggaggcacaaattcaacaagagaagaacatgtatgaattacaaaagttgaatagctgggatgtgtttggcaattggtttgaccttgcttcttggataaagtatatacaatatggagtttatatagttgtaggagtaatactgttaagaatagtgatctatatagtacaaatgctagctaagttaaggcaggggtataggccagtgttctcttccccaccctcttatttccagcagacccatatccaacaggacccggcactgccaaccagagaaggcaaagaaagagacggtggagaaggcggtggcaacagctcctggccttggcagatagaatatattcatttcctgatccgccaactgatacgcctcttgacttggctattcagcaactgcagaaccttgctatcgagagtataccagatcctccaaccaatactccagaggctctctgcgaccctacagaggattcgagaagtcctcaggactgaactgacctacctacaatatgggtggagctatttccatgaggcggtccaggccgtctggagatctgcgacagagactcttgcgggcgcgtggggagacttatgggagactcttaggagaggtggaagatggatactcgcaatccccaggaggattagacaagggcttgagctcactctcttgtgagggacagaaatacaatcagggacagtatatgaatactccatggagaaacccagctgaagagagagaaaaattagcatacagaaaacaaaatatggatgatatagatgagtaagatgatgacttggtaggggtatcagtgaggccaaaagttcccctaagaacaatgagttacaaattggcaatagacatgtctcattttataaaagaaaaggggggactggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggttctagcatggaagtttgatccaactctggcctacacttatgaggcatatgttagatacccagaagagtttggaagcaagtcaggcctgtcagaggaagaggttagaagaaggctaaccgcaagaggccttcttaacatggctgacaagaaggaaactcgctgaaacagcagggactttccacaaggggatgttacggggaggtactggggaggagccggtcgggaacgcccactttcttgatgtataaatatcactgcatttcgctctgtattcagtcgctctgcggagaggctggcagattgagccctgggaggttctctccagcactagcaggtagagcctgggtgttccctgctagactctcaccagcacttggccggtgctgggcagagtgactccacgcttgcttgcttaaagccctcttcaataaagctgccattttagaagtaagctagtgtgtgttcccatctctcctagccgccgcctggtcaactcggtactcaataataagaagaccctggtctgttaggaccctttctgctttgggaaaccgaagcaggaaaatccctagca

SEQ ID NO: 386 - DNA, 9713 bp, Human immunodeficiency virus 2
agtcgctctgcggagaggctggcagattgagccctgggaggttctctccagcactagcaggtagagcctgggtgttccctgctagactctcaccggtgcttggccggcactgggcagacggctccacgcttgcttgcttaaaagacctcttaataaagctgccagttagaagcaagttaagtgtgtgttcccatctctcctagtcgccgcctggtcattcggtgttcatctgaataacaagaccctggtctgttaggaccctttctgctttgggaaaccaaagcaggaaaatccctagcaggttggcgcccgaacagggacttagagaagactgaaaagccttggaacacggctgagtgaaggcagtaagggcggcaggaacaaaccacgacggagtgctcctagaaaggcgcaggccaaggtaccaaaggcggcgtgtggagcgggagtaaagaggcctccgggtgaaggtaagtacctacaccaaaaaattgtagccaggaagggcttgttatcctacctttagacaggtagaagattgtgggagatgggcgcgagaaactccgtcttgaaagggaaaaaagcagacgaattagaaacaattaggttacggcccggcggaaagaaaaaatacaggctaaagcatattgtgtgggcagcgaatgaattggacagattcggattagcagagagcctgttggagtcaaaagaaggttgccaaagaattcttacagttttaggtccattagtaccgacaggttcagaaaatttaaaaagcctttttaatactgtctgcgtcatttggtgcatacacgcagaagagaaagtgaaagatactgaaggagcaaaacaaatagtacagagacatctagcggcagaaacaggaactgcagagaaaatgccaaatacaagtagaccaacagcaccacctagcgggaagggaggaaacttccccgtacaacaagtaggcggcaattatacccatgtgccgctgagtcctcgaaccctaaatgcttgggtaaaattagtagaggaaaagaagttcggggcagaggtagtgccaggatttcaggcactctcagaaggctgcacgccctatgatatcaaccaaatgcttaattgtgtgggcgaccatcaagcagctatgcaaataatcagggagatcgttaatgaagaagcagcagattgggatgtgcaacatccaataccaggtcccttaccagcggggcagcttagagaaccaagagggtctgacatagcagggacaacaagcacagtagatgaacagatccagtggatgtttaggccacaaaatcccgtaccagtgggaaacatctataggagatggatccagataggactgcagaagtgcgtcaggatgtacaacccgaccaacatcctagacataaaacaaggaccaaaggaaccattccaaagttatgtagatagattctacaaaagcttgagggcagaacaaacagatccagcagtgaagaattggatgacccagacactactagtacagaatgccaacccagactgtaaattagtactaaaaggactagggatgaatcctaccttagaagagatgctaaccgcctgccaaggggtaggtgggccaggccagaaagctagactaatggcagaagccttaaaagaggccttgacaccagcccctatcccatttgcagcagcccagcagaaaaggacaattaaatgctggaattgtggaaaggaaggacactcggcaagacaatgccgagcacctagaagacagggctgctggaagtgtggtaaaccaggacatgtcatagcaaattgcccagatagacaggtgggttttttagggatgggcccccggggaaagaagccccgcaacttccccgtggcccaagtcccgcaggggctaacaccaacagcacccccagtagatccagcagtggacctactggagaattatatgcagcaaggaaaaagacaaagagaacagagagagagaccatacaaagaagtgacagaggacttactgcacctcgagcagggagaggcaccatgcagagagacgacagaggacttgctgcacctcaattctctcttttgaaaagaccagtagtcacggcatacgtcgagggccagccagtagaagttctgctagacacgggggctgacgactcaatagtagcagggatagagttagggagcaattatagtccaaagatagtaggaggaatagggggattcataaataccaaggaatataaaaatgtaaaaatagaagttttaggtaaaaaggtaagggccaccataatgacaggtgacaccccaatcaacatttttggcagaaatattctgacagccttaggcatgtcattaaatttaccagtcgccaaaatagaaccaataaaaataatgttaaagccaggaaaagatggaccaaaactgaggcaatggcccttaacaaaagaaaaaatagaggcactaaaagaaatctgtgaaaaaatggaaagagaaggccagctagaggaagcgcctccaactaatccttataacacccccacatttgcaatcaagaaaaaggacaaaaataaatggaggatgctaatagattttagagaactaaacaaggtaactcaagatttcacagaaattcagttaggaattccacacccagcaggattggccaagaaaaaaagaattactgtactagatataggggatgcttacttttccataccactacatgaagactttagacagtatactgcatttactttaccatcaataaacaatgcagaaccaggaaaaagatatatatataaggtcctgcctcagggatggaaggggtcaccagcaatttttcaatacacaatgaggcaggtcttagaaccattcagaaaagcaaacctagatgtcattatcattcagtacatggatgatatcctaatagctagtgacaggacagatctagaacatgacaaggtggtcctgcagctaaaggaacttctaaataacctaggattttctaccccagatgagaagttccaaaaggaccctccataccactggatgggctatgaactgtggccaactaagtggaagctgcagaagatacagttgccccaaaaagatgtatggacagtaaatgacatccaaaagttagtgggtgtcttaaactgggcagcacaaatctacccagggataaaaaccagacacttatgtaagctaattagaggaaaaatgacactcacagaagaagtacagtggacagaactagcagaggcggagttagaagagaacaagattatcttaagccaggagcaagagggacactattaccaagaagaaaaagagttagaagcaacagtccaaaaggatcaagacaatcagtggacatataaagtacaccagggagagaaaattctaaaagtagggaaatatgcaaagataaaaaatacccataccaatggggtcagattgttagcacaagtagttcaaaagataggaaaagaagcactaatcatttggggacgaataccaaaatttcacctaccagtagaaagagagacatgggaacagtggtgggatgactactggcaggtgacatggatccctgactgggacttcgtatctaccccgccgctggtcagactagcatttaacctggtaaaagatcctataccaagaacagagactttctacacagatggatcctgcaataggcaatcaaaggaaggaaaagcaggatatgtaacagatagagggagagacaaggtaaggatgctagaacaaactaccaatcagcaagcagaattagaagcctttgcaatggcactaacagactcaggtccaaaagccaatattatagtagactcacagtatgtaatggggatagtagcaggccagccaacagaatcagagagtagaatagtaaatcaaatcatagaggagatgataaaaaaggaagcaatctatgttgcatgggtcccagcccataaaggcataggagggaatcaggaggtagatcagttagtaagtcagggcatcagacaagtgttgttcctggaaaaaatagagcccgctcaggaagaacatgagaaataccatagcaatgtaaaagaactatcccataaatttggattgcccaaattagtagcaagacaaatagtaaacacatgtgcccaatgtcaacagaaaggggaggctatacatgggcaagtagatgcagaattaggcacttggcaaatggactgcacacacttagaaggaaagatcattatagtagcagtacatgttgcaagtggattcatagaagcagaagtcatcccacaggaatcaggaaggcagacagcactcttcctattaaaactggccagtaggtggccaataacacacttgcacacagataatggtgccaacttcacttcacaggaagtaaaaatggtagcatggtgggtaggtatagaacaatctttcggagtaccttacaatccacaaagccaaggagtagtagaagcaatgaatcaccacctaaaaaatcagataagtagaattagagaacaggcaaatacagtagaaacaatagtactgatggcaacacactgcatgaattttaaaagaaggggaggaataggggatatgaccccagcagaaagactaatcaatatgatcaccacagaacaagaaatacaattcctccacgccaaaaattcaaaattaaaaaattttcgggtctatttcagagaaggcagagatcagctgtggaaaggacccggggaactactgtggaagggagacggagcagtcatagtcaaggtagggacagacataaaagtagtaccaaggaggaaagccaagatcatcaaagactatggaggaaggcaagaactggatagtggttcccacttggagggtgccagggaggatggagaaatggcatagccttgtcaaatatctaaaatacagaacaaaagatctagaagacgtgtgctatgttccccaccataaagtaggatgggcatggtggacttgcagcagggtaatattcccattaaagggaaacagtcatctagaaatacaggcatattggaacctaacgccagaaaaaggatggctctcctcttattcagtaagaatgacttggtatacggaaaggttctggacagatgttaccccagactgtgcagactccctaatacatagcacttatttctcttgctttacagcaggtgaagtaagaagagccatcagaggggaaaagttattgtcctgctgcaattatccccaagcccatagagcccaggtaccgtcactccaatttttggccttagtggtagtgcagcaaaatgacagaccccagagaaacggtacccccaggaaacagtggcgaagagactatcgaagaggccttcaattggctagacaggacggtagaagccataaacagagaggcagtgaatcacctgccccgagagcttattttccaggtgtggcagaggtcctggagatactggcatgatgaacaagggatgtcacaaagttacacaaagtatagatatttgtgcttaatacagaaggctatgttcacacattgtaagagagggtgcacttgcctggggggaggacatgggccaggagggtggagaccaggacctccccctcctccccctccaggtctagtctaatgactgaagcaccaacagagtttcccccggaggatgggaccccaccgagggaaccaggggatgagtggataatagaaatcctgagaaaaataaagaaagaagctttaaagcattttgaccctcgcttgctaactgctcttggcaactatatccatactagacatggagacacccttgaaggcgccagagagctcattaatgtcctacaacgagccctcttcatgcacttcagagcgggatgtaggctctcaagaattggccaaacagggggaagaactcctttcccagctacatcgacccctagaaccatgcaataacaaatgctattgtaaaggatgctgcttccactgccagctgtgttttttaaacaaggggctcgggatatgttatgaccggaagggcagacgaagaagaactccgaagaaaactaaggctcattcatcttctgcatcagacaagtgagtatgatgggtggtagaaatcagctgcttgttgccattttgctaactagtacttgcttgatatattgcaccaattatgtgactgttttctatggcatacccgcgtggagaaatgcatccattcccctcttttgtgcaaccaagaatagggatacttggggaaccatacagtgcttgccagacaatgatgattatcaggagataactttgaatgtgacagaggctttcgatgcatgggataatacagtaacagaacaagcaatagaagatgtctggaatctatttgagacatcaataaaaccatgtgtcaaattaacgcctttatgtgtagcaatgagatgtaacaacacagatgcaaggaacacaaccacacccacaacagcatccccgcgtacaataaaacccgtgacagagataagtgagaattcctcatgcatacgcgcaaacaactgctcaggattgggagaagaagaggtggtcaattgtcaattcaatatgacaggattagagagagataagaaaaagcaatatagtgagacatggtactcgaaggatgtagtttgtgaaggaaatggcaccacagatacatgttacatgaaccattgcaacacatcggtcatcacagagtcatgtgacaagcactattgggatgctatgaggtttagatactgtgcaccaccaggttttgccctactaagatgcaatgataccaattattcaggctttgcgcccaattgctctaaggtagtagctgctacatgcaccagaatgatggaaacgcaaacttctacatggtttggctttaatggcactagagcagaaaatagaacatttatctattggcatggtagggataacagaactatcatcagcttaaacaaatattataatctcactatacattgtaagaggccaggaaataagacagtggtaccaataacacttatgtcagggttaaggtttcactcccagccggtcatcaataaaagacccagacaagcatggtgttggttcaaaggtgaatggaagggagccatgcaggaggtgaaggaaacccttgcaaaacatcccaggtataaaggaaccaatgaaacaaagaatattaactttacagcaccaggaaagggctcagacccagaggtggcatacatgtggactaactgcagaggagaatttctctactgcaacatgacttggttcctcaattggatagaaaataagacacaccgcaattatgtaccgtgccatataagacaaataattaacacctggcataaggtagggaaaaatgtatatttgcctcccagggaaggggagttgacctgcaactcaacagtaactagcataattgctaacattgatgcaaatggaaataatacaaatattacctttagtgcagaggtggcagaactataccgattagagttgggagattataaattggtagaaataacaccaattggcttcgcacctacagcagaaaaaagatactcctctactccaatgaggaacaagagaggtgtgttcgtgctagggttcttgggttttctcgcaacagcaggctctgcaatgggcgcggcgtccttaacgctgtcggctcagtctcggactttactggccgggatagtgcagcaacagcaacagctgttggacgtggtcaagagacaacaggaaatgttgcgactgaccgtctggggaacaaaaaatctccaggcaagagtcactgctatcgagaagtacttaaaggaccaggcgcaactaaattcatggggatgtgcatttagacaagtctgccacactactgtaccatgggtaaatgataccttaacgcctgagtggaacaatatgacgtggcaagaatgggaaggcaaaatccgcgacctggaggcaaatatcagtcaacaattagaacaagcacaaattcagcaagagaagaatatgtatgaactacaaaagttaaatagctgggatgtttttggtaactggtttgacttaacctcctggatcaagtatattcaatatggagtttatataataataggaatagtagttcttagaatagtaatatatatagtacagatgttaagtagacttagaaagggctataggcctgttttctcttccccccccggttacctccaacagatccatatccacaaggactgggaacagccagccagagaagaaacagaagaagacgttggaaacaacgttggagacagctcgtggccttggccgataagatatatacatttcctgatccaccagctgattcgcctcttggccggactatacaacatctgcaggaacttactatccaggatctccctgaccctccgaccagttttccagagtcttcagagggcactgacagcaatcagagactggctaagaactgacgcagcctacttgcagtatgggtgcgagtggatccaaggagcgttccaggccttcgcaagggctacgagagagactcttgcgggcacgtggagagacttgtggggggcactgcagcggatcgggaggggaatacttgcagtcccaagaagaatcaggcagggagcagagatcgccctcctatgagggacagcggtatcagcagggagactttatgaataccccatggagaaccccagcaaaagaaggggagaaagaattgtacaagcaacaaaatagagatgatgtagattcggatgatgatgacctagtaggggtctctgtcacaccaagagtaccactaagagaattgacacatagattagcaatagatgtgtcacattttataaaagaaaaagggggactggaagggatgtattacagtgagagaagacatagaatcttagacatataccttgaaaaggaagaagggataattgcagattggcagaactatactcatgggccaggaataagatacccaatgttctttgggtggctatggaagctagtaccagtagatgtcacacgacaggaggaggacgatgggactcactgtttactacacccagcacaaacaagcaggtttgatgacccgcatggggaaacactgatatggaagtttgaccccacgctggctcatgattacaaggcttttatcctgcacccagaggaatttgggcataagtcaggcctgccagaagaagactggaaggcaagactgaaagcaagagggataccatttagttagagacaggaacagctatatttggccagggcaggaaataactactgaaaacagctgagactgcagggactttccgaaggggctgtaaccaggggagggacatgggaggagccggtggggaacgccctcatactttctgtataaagatacccgctgcttgcattgtacttcagtcgctctgcggagaggctggcagattgagccctgggaggttctctccagcactagcaggtagagcctgggtgttccctgctagactctcaccggtgcttggccggcactgggcagacggctccacgcttgcttgcttaaaagacctcttaataaagctgccagttagaagcaagttaagtgtgtgttcccatctctcctagtcgccgcctggtc

SEQ ID NO: 387 - DNA, 11878 bp, Artificial Sequence
  Description of Artificial Sequence Synthetic polynucleotide
gcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttggggggatcctctagagtcgacctgcaggcatgctatttgatgaattaactacacttaaaataatacaattattattaaatttttttttgatttatttattaatttttaaacttaatcatttgtatttgggaggaattatatatatctttataattattttatttttttttatttttttatttttttattattattatttttttttattttttttttttactgtatcaaagaaaaacctttaaaaaaaaaattataatttccccatcttactatatttttaatacatacgttttaaggaattaaattagacaaaagctatattatgctttacatataattagaatttataaacgtttggttattagatatttcatgtctcagtaaagtctttcaatacatatgtaaaaaaatatatatgaatacacataagttgttaatatattttatatgcataaatgtataaatatatatatatatatatatatatgtatgtatgtatatgtgtgtatatgaaattatttcaatgtttaattttttaaattttaatttttttttttttttttttttttattatgtatattgatctttattatttaaatattacttttttcgttttttcttctttttattatttttttttttttttatattttatacaaatggtaattcaaataaaaggtataaatttatatttaattttcttttatggataaataaaagaaaaatataaatatataaaaatataaaaatatatatatgtatattggggtgatgataaaatgaaagataatatatatatatatatatctttatttttttttttttgtagaccccattgtgagtacataaatatattatataactcgggagcatcagtcatggaattcttatttctttttcttttttgcctggccggcctttttcgtggccgccggccttttgtcgcctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatatggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatgtccaggccgatgctgtacttcttgtcggctgctgggactccgtggataccgaccttccgcttcttctttggggccatcttatcgtcatcgtctttgtaatcaatatcatgatccttgtagtctccgtcgtggtccttatagtccatttttctcgagggatcctgatatatttctattaggtatttattattataaaatataaatcttgaatgataataaataaaatattagttattccttttctagtttaaaatatacatattataaatatatatatatatatatatatttttattgtgacaagaatatataattataaattatattatttatttttgtatttttttttttttttttttttttttctttttttgttttatttttctttttttttataaatattatttttttcttttatcatgcacattggaataatacattaatatatatatatatattatattatacatatattgaataatgtttataaaaaatgcataacttatatgaatataattttttttaaatatgacaaaaagaaaaaaaaaaaaaaccaaaaaaaattaaaattgaaatgaaatatataaatatattatttatatatattatacattgtttaatactactacatgtatatatatatattatatatatatatatatatcaattttttcaaaaataaattaatataaaaagaggggaaaaaaaaaaaaaaaaaaaaaaaaagataattaagtaagcatttaaaaatatataaattgataatatataaaattaatcacatataaaagcttataaacactaggttagctaattcgcttgtaagaggtactctcgtttatgcaaaactatttgatatagcattttaacaagtacacatatatatatgtaatatatatactatatatatctattgcatgtgtactaagcatgtgcatggcatcccctttttctcgtgtttaaaacagtttgtatgataaaatataaaggatttgaaaaagagaaaaaaatatatgatctcatcctatatagcgccataatttttatttgggttgaataaaattttctactaaatttaggtgtaagtaaaataatggaatatatataagtacaataaaaaagtgcataaattaaaaaatttttataataaatattttttttaaaaaagtcaataataatattaaatatatataacacaggattatatatgttcactacaattttttatattataatataaattcttttcaattttcattttattttacatacactttccttttttgtcactatattttaatattcacatatttagtttaaatactggctatttctttctacatttgctagtaacaattgtgtagtgcttaaatatatacacacacctaaaacttacaaagtatcctaggaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaccgcgggtaccccattaaatttatttaataatagattaaaaatattataaaaataaaaacataaacacagaaattacaaaaaaaatacatatgaattttttttttgtaatcttccttataaatatagaataatgaatcatataaaacatatcattattcatttatttacatttaaaattattgtttcagtatctttaatttattatgtatatataaaaataacttacaattttattaataaacaatatatgtttattaattcatgttttgtaatttatgggatagcgattttttttactgtctgtattttcttttttaattatgttttaattgtatttattttatttttattattgttctttttatagtattattttaaaacaaaatgtattttctaagaacttataataataataatataaattttaataaaaattatatttatcttttacaatatgaacataaagtacaacattaatatatagcttttaatatttttattcctaatcatgtaaatcttaaatttttctttttaaacatatgttaaatatttatttctcattatatataagaacatatttattacatctagaggtaccgagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggcctaacattagtaatgtaggtctgactttcactcatataagtcttatggtaactaaactaaggtcttacctttactgatatatgtcttactttcactaacttaggtattacttttactaacttaggtcttaaattcagtaactaaggtcatacttcgactaactaaggtcttacattcactgatataggtcttatgattactaacttaggtcctaatttgactaacataagtcctaacattagtaatgtaggtcttaacttaactaacttaggtcttaccttcactaatataggtcttaatattactgacttaagtaattaaggtactaacttaggtcgtaaggtaactaatatataggtcttaaggtaactaatttaggtcttgacttaataaatataggtcctaacataaatagtataggtcctaatataagtactataggccttaacttaaccaacataggtcctaacataagttatataggtcttaacgtaactaacataagtcattaaggtactaagtttggtcttaatttaacaataacatgtcgctggcctaacattagtaatgtaggtctgactttcactcatataagtcttatggtaactaaactaaggtcttacctttactgatatatgtcttactttcactaacttaggtattacttttactaacttaggtcttaaattcagtaactaaggtcatacttcgactaactaaggtcttacattcactgatataggtcttatgattactaacttaggtcctaatttgactaacataagtcctaacattagtaatgtaggtcttaacttaactaacttaggtcttaccttcactaatataggtcttaatattactgacttaagtaattaaggtactaacttaggtcgtaaggtaactaatatataggtcttaaggtaactaatttaggtcttgacttaataaatataggtcctaacataaatagtataggtcctaatataagtactataggccttaacttaaccaacataggtcctaacataagttatataggtcttaacgtaactaacataagtcattaaggtactaagtttggtcttaatttaacaataaccatgtcgctggccgggtggtcttaatttaacaaatatagaccatgtcgctggccgggtgacccggcggggacgaggcaagctaaacagatcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatctcgacctgcagcgtacgaagct

SEQ ID NO: 388 - DNA, 12044 bp, Artificial Sequence
  Description of Artificial Sequence Synthetic polynucleotide
gcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttggggggatcctctagagtcgactaatacgactcactataggaacataatctatagcggcgttttagagctagaaatagcaagttaaaataaggctagtccgttatcaacttgaaaaagtggcaccgagtcggtgctagcataaccccttggggcctctaaacgggtcttgaggggttttttggtcgacctgcaggcatgctatttgatgaattaactacacttaaaataatacaattattattaaatttttttttgatttatttattaatttttaaacttaatcatttgtatttgggaggaattatatatatctttataattattttatttttttttatttttttatttttttattattattatttttttttattttttttttttactgtatcaaagaaaaacctttaaaaaaaaaattataatttccccatcttactatatttttaatacatacgttttaaggaattaaattagacaaaagctatattatgctttacatataattagaatttataaacgtttggttattagatatttcatgtctcagtaaagtctttcaatacatatgtaaaaaaatatatatgaatacacataagttgttaatatattttatatgcataaatgtataaatatatatatatatatatatatatgtatgtatgtatatgtgtgtatatgaaattatttcaatgtttaattttttaaattttaatttttttttttttttttttttttattatgtatattgatctttattatttaaatattacttttttcgttttttcttctttttattatttttttttttttttatattttatacaaatggtaattcaaataaaaggtataaatttatatttaattttcttttatggataaataaaagaaaaatataaatatataaaaatataaaaatatatatatgtatattggggtgatgataaaatgaaagataatatatatatatatatatctttatttttttttttttgtagaccccattgtgagtacataaatatattatataactcgggagcatcagtcatggaattcttatttctttttcttttttgcctggccggcctttttcgtggccgccggccttttgtcgcctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatatggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatgtccaggccgatgctgtacttcttgtcggctgctgggactccgtggataccgaccttccgcttcttctttggggccatcttatcgtcatcgtctttgtaatcaatatcatgatccttgtagtctccgtcgtggtccttatagtccatttttctcgagggatcctgatatatttctattaggtatttattattataaaatataaatcttgaatgataataaataaaatattagttattccttttctagtttaaaatatacatattataaatatatatatatatatatatatttttattgtgacaagaatatataattataaattatattatttatttttgtatttttttttttttttttttttttttctttttttgttttatttttctttttttttataaatattatttttttcttttatcatgcacattggaataatacattaatatatatatatatattatattatacatatattgaataatgtttataaaaaatgcataacttatatgaatataattttttttaaatatgacaaaaagaaaaaaaaaaaaaaccaaaaaaaattaaaattgaaatgaaatatataaatatattatttatatatattatacattgtttaatactactacatgtatatatatatattatatatatatatatatatcaattttttcaaaaataaattaatataaaaagaggggaaaaaaaaaaaaaaaaaaaaaaaaagataattaagtaagcatttaaaaatatataaattgataatatataaaattaatcacatataaaagcttataaacactaggttagctaattcgcttgtaagaggtactctcgtttatgcaaaactatttgatatagcattttaacaagtacacatatatatatgtaatatatatactatatatatctattgcatgtgtactaagcatgtgcatggcatcccctttttctcgtgtttaaaacagtttgtatgataaaatataaaggatttgaaaaagagaaaaaaatatatgatctcatcctatatagcgccataatttttatttgggttgaataaaattttctactaaatttaggtgtaagtaaaataatggaatatatataagtacaataaaaaagtgcataaattaaaaaatttttataataaatattttttttaaaaaagtcaataataatattaaatatatataacacaggattatatatgttcactacaattttttatattataatataaattcttttcaattttcattttattttacatacactttccttttttgtcactatattttaatattcacatatttagtttaaatactggctatttctttctacatttgctagtaacaattgtgtagtgcttaaatatatacacacacctaaaacttacaaagtatcctaggaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaccgcgggtaccccattaaatttatttaataatagattaaaaatattataaaaataaaaacataaacacagaaattacaaaaaaaatacatatgaattttttttttgtaatcttccttataaatatagaataatgaatcatataaaacatatcattattcatttatttacatttaaaattattgtttcagtatctttaatttattatgtatatataaaaataacttacaattttattaataaacaatatatgtttattaattcatgttttgtaatttatgggatagcgattttttttactgtctgtattttcttttttaattatgttttaattgtatttattttatttttattattgttctttttatagtattattttaaaacaaaatgtattttctaagaacttataataataataatataaattttaataaaaattatatttatcttttacaatatgaacataaagtacaacattaatatatagcttttaatatttttattcctaatcatgtaaatcttaaatttttctttttaaacatatgttaaatatttatttctcattatatataagaacatatttattacatctagaggtaccgagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggcctaacattagtaatgtaggtctgactttcactcatataagtcttatggtaactaaactaaggtcttacctttactgatatatgtcttactttcactaacttaggtattacttttactaacttaggtcttaaattcagtaactaaggtcatacttcgactaactaaggtcttacattcactgatataggtcttatgattactaacttaggtcctaatttgactaacataagtcctaacattagtaatgtaggtcttaacttaactaacttaggtcttaccttcactaatataggtcttaatattactgacttaagtaattaaggtactaacttaggtcgtaaggtaactaatatataggtcttaaggtaactaatttaggtcttgacttaataaatataggtcctaacataaatagtataggtcctaatataagtactataggccttaacttaaccaacataggtcctaacataagttatataggtcttaacgtaactaacataagtcattaaggtactaagtttggtcttaatttaacaataacatgtcgctggcctaacattagtaatgtaggtctgactttcactcatataagtcttatggtaactaaactaaggtcttacctttactgatatatgtcttactttcactaacttaggtattacttttactaacttaggtcttaaattcagtaactaaggtcatacttcgactaactaaggtcttacattcactgatataggtcttatgattactaacttaggtcctaatttgactaacataagtcctaacattagtaatgtaggtcttaacttaactaacttaggtcttaccttcactaatataggtcttaatattactgacttaagtaattaaggtactaacttaggtcgtaaggtaactaatatataggtcttaaggtaactaatttaggtcttgacttaataaatataggtcctaacataaatagtataggtcctaatataagtactataggccttaacttaaccaacataggtcctaacataagttatataggtcttaacgtaactaacataagtcattaaggtactaagtttggtcttaatttaacaataaccatgtcgctggccgggtggtcttaatttaacaaatatagaccatgtcgctggccgggtgacccggcggggacgaggcaagctaaacagatcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatctcgacctgcagcgtacgaagct

SEQ ID NO: 389 - DNA, 12044 bp, Artificial Sequence
  Description of Artificial Sequence Synthetic polynucleotide
gcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttggggggatcctctagagtcgactaatacgactcactataggaaatgatatggattttgggttttagagctagaaatagcaagttaaaataaggctagtccgttatcaacttgaaaaagtggcaccgagtcggtgctagcataaccccttggggcctctaaacgggtcttgaggggttttttggtcgacctgcaggcatgctatttgatgaattaactacacttaaaataatacaattattattaaatttttttttgatttatttattaatttttaaacttaatcatttgtatttgggaggaattatatatatctttataattattttatttttttttatttttttatttttttattattattatttttttttattttttttttttactgtatcaaagaaaaacctttaaaaaaaaaattataatttccccatcttactatatttttaatacatacgttttaaggaattaaattagacaaaagctatattatgctttacatataattagaatttataaacgtttggttattagatatttcatgtctcagtaaagtctttcaatacatatgtaaaaaaatatatatgaatacacataagttgttaatatattttatatgcataaatgtataaatatatatatatatatatatatatgtatgtatgtatatgtgtgtatatgaaattatttcaatgtttaattttttaaattttaatttttttttttttttttttttttattatgtatattgatctttattatttaaatattacttttttcgttttttcttctttttattatttttttttttttttatattttatacaaatggtaattcaaataaaaggtataaatttatatttaattttcttttatggataaataaaagaaaaatataaatatataaaaatataaaaatatatatatgtatattggggtgatgataaaatgaaagataatatatatatatatatatctttatttttttttttttgtagaccccattgtgagtacataaatatattatataactcgggagcatcagtcatggaattcttatttctttttcttttttgcctggccggcctttttcgtggccgccggccttttgtcgcctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatatggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatgtccaggccgatgctgtacttcttgtcggctgctgggactccgtggataccgaccttccgcttcttctttggggccatcttatcgtcatcgtctttgtaatcaatatcatgatccttgtagtctccgtcgtggtccttatagtccatttttctcgagggatcctgatatatttctattaggtatttattattataaaatataaatcttgaatgataataaataaaatattagttattccttttctagtttaaaatatacatattataaatatatatatatatatatatatttttattgtgacaagaatatataattataaattatattatttatttttgtatttttttttttttttttttttttttctttttttgttttatttttctttttttttataaatattatttttttcttttatcatgcacattggaataatacattaatatatatatatatattatattatacatatattgaataatgtttataaaaaatgcataacttatatgaatataattttttttaaatatgacaaaaagaaaaaaaaaaaaaaccaaaaaaaattaaaattgaaatgaaatatataaatatattatttatatatattatacattgtttaatactactacatgtatatatatatattatatatatatatatatatcaattttttcaaaaataaattaatataaaaagaggggaaaaaaaaaaaaaaaaaaaaaaaaagataattaagtaagcatttaaaaatatataaattgataatatataaaattaatcacatataaaagcttataaacactaggttagctaattcgcttgtaagaggtactctcgtttatgcaaaactatttgatatagcattttaacaagtacacatatatatatgtaatatatatactatatatatctattgcatgtgtactaagcatgtgcatggcatcccctttttctcgtgtttaaaacagtttgtatgataaaatataaaggatttgaaaaagagaaaaaaatatatgatctcatcctatatagcgccataatttttatttgggttgaataaaattttctactaaatttaggtgtaagtaaaataatggaatatatataagtacaataaaaaagtgcataaattaaaaaatttttataataaatattttttttaaaaaagtcaataataatattaaatatatataacacaggattatatatgttcactacaattttttatattataatataaattcttttcaattttcattttattttacatacactttccttttttgtcactatattttaatattcacatatttagtttaaatactggctatttctttctacatttgctagtaacaattgtgtagtgcttaaatatatacacacacctaaaacttacaaagtatcctaggaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaccgcgggtaccccattaaatttatttaataatagattaaaaatattataaaaataaaaacataaacacagaaattacaaaaaaaatacatatgaattttttttttgtaatcttccttataaatatagaataatgaatcatataaaacatatcattattcatttatttacatttaaaattattgtttcagtatctttaatttattatgtatatataaaaataacttacaattttattaataaacaatatatgtttattaattcatgttttgtaatttatgggatagcgattttttttactgtctgtattttcttttttaattatgttttaattgtatttattttatttttattattgttctttttatagtattattttaaaacaaaatgtattttctaagaacttataataataataatataaattttaataaaaattatatttatcttttacaatatgaacataaagtacaacattaatatatagcttttaatatttttattcctaatcatgtaaatcttaaatttttctttttaaacatatgttaaatatttatttctcattatatataagaacatatttattacatctagaggtaccgagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggcctaacattagtaatgtaggtctgactttcactcatataagtcttatggtaactaaactaaggtcttacctttactgatatatgtcttactttcactaacttaggtattacttttactaacttaggtcttaaattcagtaactaaggtcatacttcgactaactaaggtcttacattcactgatataggtcttatgattactaacttaggtcctaatttgactaacataagtcctaacattagtaatgtaggtcttaacttaactaacttaggtcttaccttcactaatataggtcttaatattactgacttaagtaattaaggtactaacttaggtcgtaaggtaactaatatataggtcttaaggtaactaatttaggtcttgacttaataaatataggtcctaacataaatagtataggtcctaatataagtactataggccttaacttaaccaacataggtcctaacataagttatataggtcttaacgtaactaacataagtcattaaggtactaagtttggtcttaatttaacaataacatgtcgctggcctaacattagtaatgtaggtctgactttcactcatataagtcttatggtaactaaactaaggtcttacctttactgatatatgtcttactttcactaacttaggtattacttttactaacttaggtcttaaattcagtaactaaggtcatacttcgactaactaaggtcttacattcactgatataggtcttatgattactaacttaggtcctaatttgactaacataagtcctaacattagtaatgtaggtcttaacttaactaacttaggtcttaccttcactaatataggtcttaatattactgacttaagtaattaaggtactaacttaggtcgtaaggtaactaatatataggtcttaaggtaactaatttaggtcttgacttaataaatataggtcctaacataaatagtataggtcctaatataagtactataggccttaacttaaccaacataggtcctaacataagttatataggtcttaacgtaactaacataagtcattaaggtactaagtttggtcttaatttaacaataaccatgtcgctggccgggtggtcttaatttaacaaatatagaccatgtcgctggccgggtgacccggcggggacgaggcaagctaaacagatcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatctcgacctgcagcgtacgaagct